(19) United States
(12) Patent Application Publication
(10) Pub. No.: US 2005/0171350 A1
(43) Pub. Date: Aug. 4, 2005

(54) Benzhydryl derivatives

(75) Inventors: Kazuhiko Take, Osaka (JP); Chiyoshi Kasahara, Osaka (JP); Shinji Shigenaga, Osaka (JP); Yoshiteru Eikyu, Osaka (JP); Takashi Tojo, Osaka (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Fujisawa Pharamaceutical Co., Ltd., Osaka (JP)

(21) Appl. No.: 10/498,018

(22) PCT Filed: Dec. 16, 2002
(86) PCT No.: PCT/JP02/13156

(30) Foreign Application Priority Data
Dec. 21, 2001 (AU) PR9707

(51) Int. Cl. C07D 487/02; C07D 43/02

(52) U.S. Cl. 544/295

(57) ABSTRACT

A compound of the formula (I): 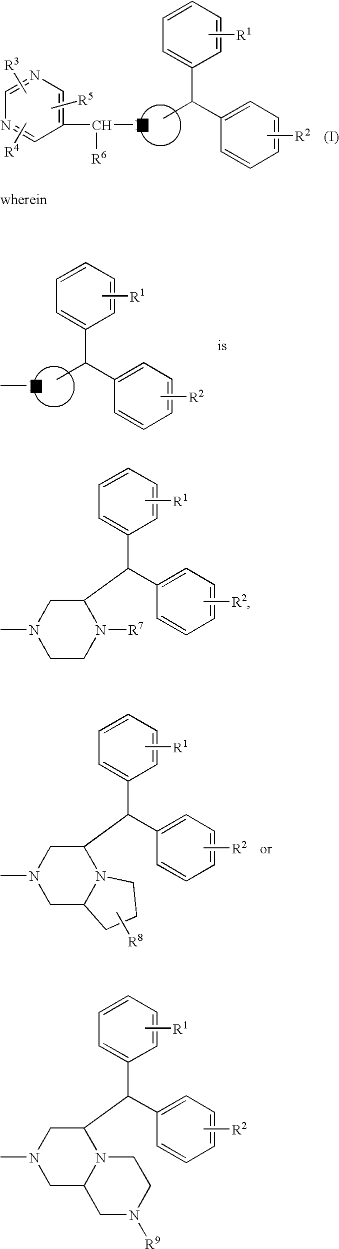
in which R¹, R², R³, R⁴, R⁵, R⁶, R⁷, R⁸ and R⁹ are each as defined in the description, or a salt thereof. The object compound of the present invention has pharmacological activities such as Tachykinin antagonism, and is useful for manufacture of a medicament for treating or preventing Tachykinin-mediated diseases.

### TECHNICAL FIELD

The present invention relates to new benzhydryl derivatives and a salt thereof.

More particularly, it relates to new benzhydryl derivatives and a salt thereof which have pharmacological activities such as Tachykinin antagonism, especially Substance P antagonism, Neurokinin A antagonism, Neurokinin B antagonism, and the like, to a process for preparation thereof, to a pharmaceutical composition comprising the same, and to a use of the same as a medicament.

Accordingly, one object of the present invention is to provide new and useful benzhydryl derivatives and a salt thereof which have pharmacological activities such as Tachykinin antagonism, especially Substance P antagonism, Neurokinin A antagonism, Neurokinin B antagonism, and the like.

Another object of the present invention is to provide a process for the preparation of said benzhydryl derivatives and a salt thereof.

A further object of the present invention is to provide a pharmaceutical composition comprising, as an active ingredient, said benzhydryl derivatives and a pharmaceutically acceptable salt thereof.

Still further object of the present invention is to provide a use of said benzhydryl derivatives or a pharmaceutically acceptable salt thereof as Tachykinin antagonist, especially Substance P antagonist, Neurokinin A antagonist or Neurokinin B antagonist, useful for treating or preventing Tachykinin-mediated diseases, for example, respiratory diseases such as asthma, bronchitis, rhinitis, cough, expectoration, and the like; ophthalmic diseases such as conjunctivitis, vernal conjunctivitis, and the like; cutaneous diseases such as contact dermatitis, atopic dermatitis, urticaria, and other eczematoid dermatitis, and the like; inflammatory diseases such as rheumatoid arthritis, osteoarthritis, and the like; pains or aches (e.g., migraine, headache, toothache, cancerous pain, back pain, etc.); and the like in human being or animals.

### BACKGROUND ART

Some benzhydryl derivatives have been known as described in, for example, WO 02/00631 and WO 02/055518.

### DISCLOSURE OF INVENTION

The object compound of the present invention can be represented by the following general formula (I): 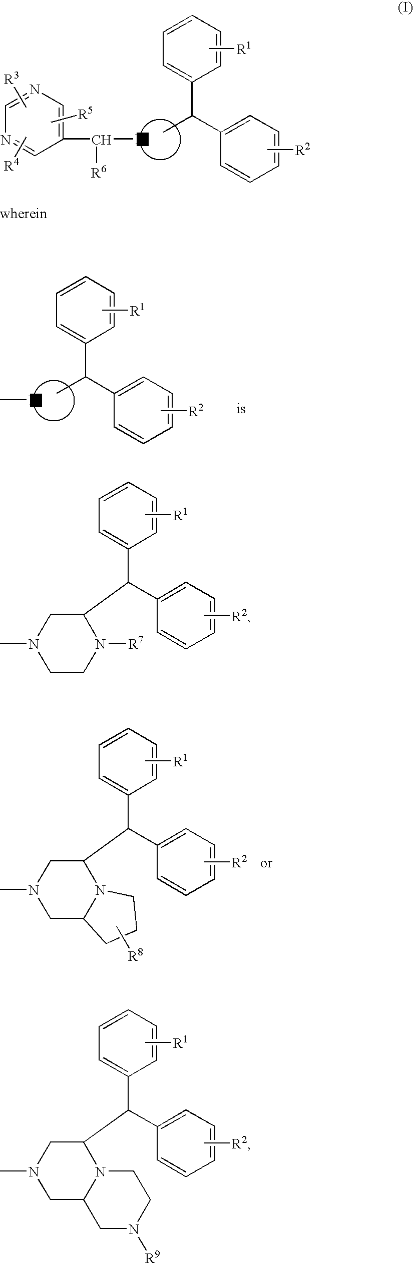
in which
R¹ and R² are independently hydrogen, halogen or lower alkyl,
R⁷ is hydrogen, lower alkoxycarbonyl or pyrazolyl(lower)alkyl optionally substituted with lower alkyl,
R⁸ is hydrogen or lower alkanoylamino,
R⁹ is hydrogen; lower alkanoyl optionally substituted with one, two or three substituent(s) selected from a group consisting of hydroxy, lower alkoxy, halogen, hydroxy(lower)alkyl, acetylamino, mono(or di)(lower)alkylamino and pyridyl; di(lower)alkylcarbamoyl; bis(hydroxy(lower)alkyl)carbamoyl; benzyloxycarbonyl; benzoyl; or heterocycliccarbonyl optionally substituted with one or two substituent(s) selected from a group consisting of hydroxy, lower alkyl, lower alkoxy, amino, carbamoyl and oxido,
R³, R⁴ and R⁵ are independently hydrogen; halogen; lower alkyl; cyclo(lower)alkyl; cyclo(lower)alkyloxy; lower alkoxy optionally substituted with one, two or three halogen(s); or lower alkanoyl optionally substituted with one, two or three halogen(s), and
R⁶ is hydrogen or lower alkyl.

It is to be noted that the object compound (I) may include one or more stereoisomers due to asymmetric carbon atom(s) and double bond, and all of such isomers and a mixture thereof are included within the scope of the present invention.

It is further to be noted that isomerization or rearrangement of the object compound (I) may occur due to the effect of the light, acid, base or the like, and the compound obtained as the result of said isomerization or rearrangement is also included within the scope of the present invention.

It is also to be noted that the solvating form of the compound (I) (e.g. hydrate, etc.) and any form of the crystal of the compound (I) are included within the scope of the present invention.

According to the present invention, the object compound (I) or a salt thereof can be prepared by processes which are illustrated in the following schemes. 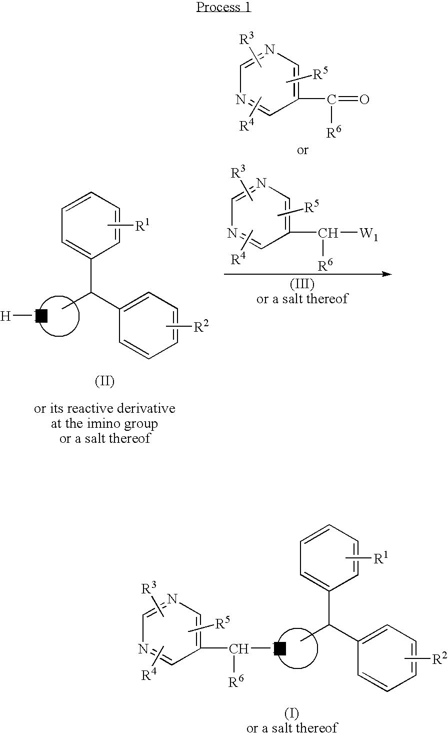 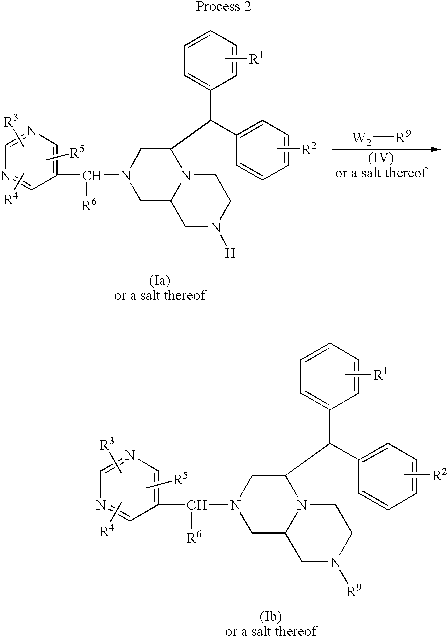 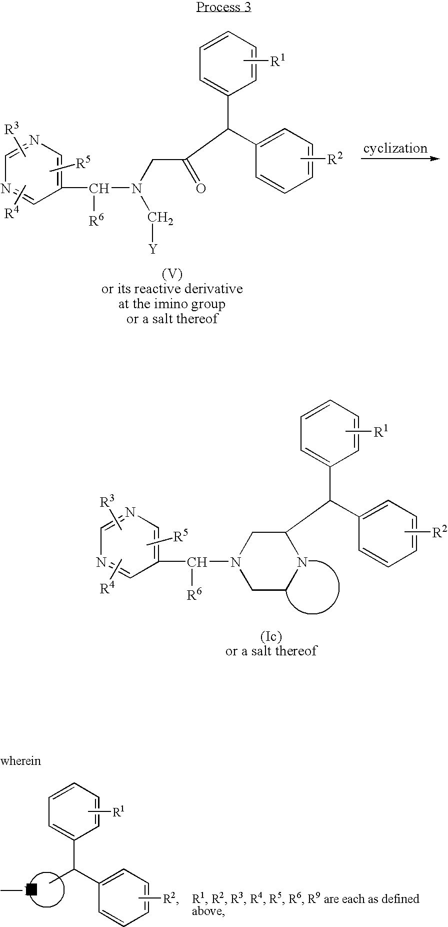
W₁ and W₂ are each a leaving group,
Y is 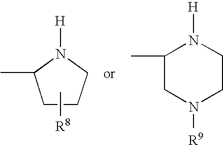
   or its reactive derivative at the imino group [in which R⁸ and R⁹ are each as defined above], and 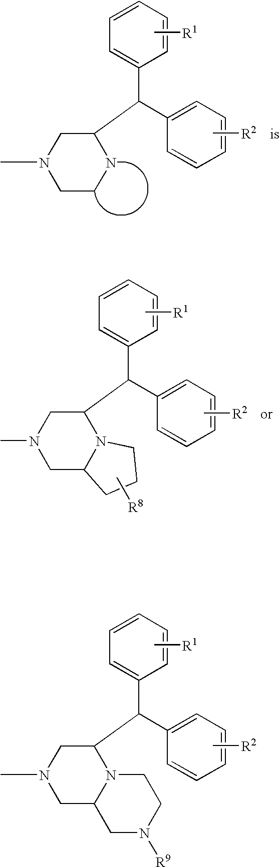
   [in which R¹, R², R⁸ and R⁹ are each as defined above].

As to the starting compounds (II), (III), (IV) and (V), some of them are novel and can be prepared by the procedures described in the Preparations and Examples mentioned later or similar manners thereto.

Suitable salts of the starting and object compounds are conventional non-toxic and pharmaceutically acceptable salt and include an acid addition salt such as an organic acid salt (e.g. acetate, trifluoroacetate, fumarate, maleate, tartrate, methanesulfonate, benzenesulfonate, formate, toluenesulfonate, etc.), an inorganic acid salt (e.g. hydrochloride, hydrobromide, hydroiodide, sulfate, nitrate, phosphate, etc.), or a salt with an amino acid (e.g. arginine, aspartic acid, glutamic acid, etc.), or a metal salt such as an alkali metal salt (e.g. sodium salt, potassium salt, etc.) and an alkaline earth metal salt (e.g. calcium salt, magnesium salt, etc.), an ammonium salt, an organic base salt (e.g. trimethylamine salt, triethylamine salt, pyridine salt, picoline salt, dicyclohexylamine salt, N,N′-dibenzylethylenediamine salt, etc.), or the like.

In the above and subsequent descriptions of the present specification, suitable examples and illustrations of the various definitions which the present invention intends to include within the scope thereof are explained in detail as follows.

The term “lower” is intended to mean 1 to 6, preferably 1 to 4, carbon atom(s), unless otherwise indicated.

Suitable “halogen” may include fluorine, chlorine, bromine and iodine.

Suitable “lower alkyl” and “lower alkyl” moiety in the terms of “pyrazolyl(lower)alkyl”, “hydroxy(lower)alkyl”, etc. may include straight or branched one having 1 to 6 carbon atom(s), such as methyl, ethyl, propyl, isopropyl, butyl, isobutyl, pentyl, hexyl and the like, in which the preferred one is C₁-C₄ alkyl and the most preferred one is methyl, ethyl, isopropyl or isobutyl.

Suitable “cyclo(lower)alkyl” and “cyclo(lower)alkyl” moiety in the term of “cyclo(lower)alkyloxy” may include cyclopropyl, cyclobutyl, cyclopentyl, cyclohexyl, cycloheptyl and the like, in which the preferred one is cyclo(C₃-C₆)alkyl and the most preferred one is cyclopropyl or cyclobutyl.

Suitable “lower alkoxy” and “lower alkoxy” moiety in the term of “lower alkoxycarbonyl” may include methoxy, ethoxy, propoxy, isopropoxy, butoxy, isobutoxy, t-butoxy, pentyloxy, t-pentyloxy, hexyloxy and the like, in which the preferred one is C₁-C₄ alkoxy and the most preferred one is methoxy, ethoxy, isopropoxy or t-butoxy.

Suitable “lower alkanoyl” and “lower alkanoyl” moiety in the term of “lower alkanoylamino” may include formyl, acetyl, propanoyl, butanoyl, 2-methylpropanoyl, pentanoyl, 2,2-dimethylpropanoyl, hexanoyl and the like, in which the preferred one is C₁-C₄ alkanoyl and the most preferred one is acetyl or propanoyl.

Suitable “heterocyclic” moiety in the term of “heterocycliccarbonyl” may be saturated or unsaturated monocyclic or polycyclic heterocyclic group containing at least one hetero atom selected from among oxygen, sulfur and nitrogen.

The particularly preferred example of said heterocyclic moiety may include unsaturated 3- through 8-membered heteromonocyclic groups containing 1 through 4 nitrogen atom(s), such as pyrrolyl, pyrrolinyl, imidazolyl, pyrazolyl, pyridyl, pyrimidyl, pyrazinyl, pyridazinyl, triazolyl (e.g. 4H-1,2,4-triazolyl, 1H-1,2,3-triazolyl, 2H-1,2,3-triazolyl, etc.), tetrazolyl (e.g. 1H-tetrazolyl, 2H-tetrazolyl, etc.), dihydrotriazinyl (e.g. 4,5-dihydro-1,2,4-triazinyl, 2,5-dihydro-1,2,4-triazinyl, etc.), etc.;
3- through 8-membered saturated heteromonocyclic groups containing 1 through 4 nitrogen atom(s), such as pyrrolidinyl, imidazolidinyl, piperidyl (e.g. piperidino, etc.), piperazinyl, etc.;
unsaturated condensed heterocyclic groups containing 1 through 5 nitrogen atom(s), such as indolyl, isoindolyl, indolizinyl, benzimidazolyl, quinolyl, isoquinolyl, indazolyl, benzotriazolyl, tetrazolopyridyl, tetrazolopyridazinyl (e.g. tetrazolo[1,5-b]pyridazinyl, etc.), dihydrotriazolopyridazinyl, etc.;
3- through 8-membered unsaturated heteromonocyclic groups containing 1 or 2 oxygen atoms and 1 through 3 nitrogen atom(s), such as oxazolyl, isoxazolyl, oxadiazolyl (e.g. 1,2,4-oxadiazolyl, 1,3,4-oxadiazolyl, 1,2,5-oxadiazolyl, etc.), etc.;
3- through 8-membered saturated heteromonocyclic groups containing 1 or 2 oxygen atom(s) and 1 through 3 nitrogen atoms, such as morpholinyl, oxazolidinyl (e.g. 1,3-oxazolidinyl, etc.), etc.;
unsaturated condensed heterocyclic groups containing 1 or 2 oxygen atom(s) and 1 through 3 nitrogen atom(s), such as benzoxazolyl, benzoxadiazolyl, etc.;
3- through 8-membered unsaturated heteromonocyclic groups containing 1 or 2 sulfur atom(s) and 1 through 3 nitrogen atom(s), such as 1,3-thiazolyl, 1,2-thiazolyl, thiazolinyl, thiadiazolyl (e.g. 1,2,4-thiadiazolyl, 1,3,4-thiadiazolyl, 1,2,5-thiadiazolyl, 1,2,3-thiadiazolyl), etc.;
3 through 8-membered saturated heteromonocyclic groups containing 1 or 2 Sulfur atom(s) and 1 through 3 nitrogen atom(s), such as thiazolidinyl, etc.;
3- through 8-membered unsaturated heteromonocyclic groups containing 1 Sulfur atom, such as thienyl, etc.;
unsaturated condensed heterocyclic groups containing 1 or 2 Sulfur atoms and 1 through 3 nitrogen atom(s), such as benzothiazolyl, benzothiadiazolyl, etc.;
3- through 8-membered unsaturated heteromonocyclic groups containing 1 or 2 oxygen atoms(s), such as furyl, pyranyl, dioxolyl, etc.;
3- through 8-membered saturated heteromonocyclic groups containing 1 or 2 oxygen atom(s), such as oxetanyl, oxolanyl, tetrahydropyranyl (e.g. tetrahydro-2H-pyran-4-yl, etc.), dioxolanyl, dioxanyl (e.g. 1,4-dioxan-2-yl, 1,3-dioxan-5-yl, etc.), etc.; and
unsaturated condensed heterocyclic groups ontaining 1 or 2 oxygen atom(s), such as isobenzofuranyl, chromenyl (e.g. 2H-chromen-3-yl, etc.), dihydrochromenyl (e.g. 3,4-dihydro-2H-chromen-4-yl, etc.), etc.

Suitable “N-containing heterocyclic group” may be aforesaid “heterocyclic group”, in which said group contains at least one N atom in its ring members.

The more preferred one may include 5- through 6-membered heteromonocyclic groups containing 1 or 2 hetero atom(s), such as pyridinyl, furyl, thienyl, thiazolyl or morpholino.

Suitable “leaving group” may include lower alkoxy (e.g. methoxy, ethoxy, propoxy, isopropoxy, butoxy, isobutoxy, t-butoxy, pentoxy, etc.), aryloxy (e.g., phenoxy, naphthoxy, etc.), an acid residue or the like.

Suitable “acid residue” may be halogen (e.g., chlorine, bromine, iodine, etc.), sulfonyloxy (e.g., methanesulfonyloxy, phenylsulfonyloxy, mesitylenesulfonyloxy, toluenesulfonyloxy, etc.) or the like.

Preferred embodiments of the object compound (I) are as follows: 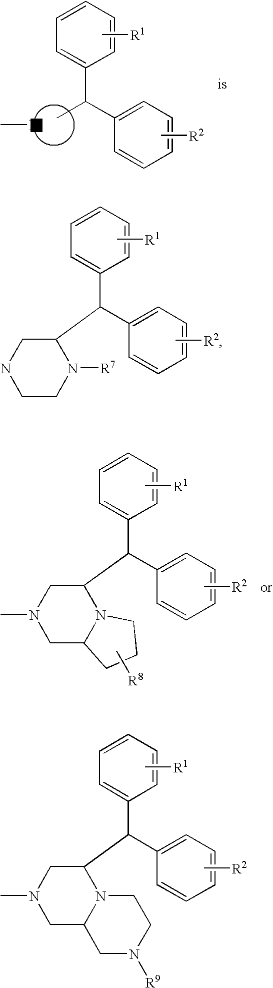
in which
R¹ and R² are independently hydrogen, halogen (more preferably fluoro) or lower alkyl (more preferably C₁-C₄ alkyl, most preferably methyl),
R⁷ is hydrogen, lower alkoxycarbonyl or pyrazolyl(lower)alkyl optionally substituted with lower alkyl,
R⁸ is hydrogen or lower alkanoylamino,
R⁹ is hydrogen; lower alkanoyl (more preferably C₁-C₄ alkanoyl, most preferably acetyl, propionyl or butyryl) optionally substituted with one, two or three substituent(s) selected from a group consisting of hydroxy, halogen (more preferably fluoro), lower alkoxy (more preferably C₁-C₄ alkoxy, most preferably methoxy), hydroxy(lower)alkyl (more preferably hydroxy-(C₁-C₄)alkyl, most preferably hydroxymethyl), acetylamino, mono(or di)(lower)alkylamino (more preferably mono(or di)(C₁-C₄)alkylamino, most preferably dimethylamino) and pyridyl; di(lower)alkylcarbamoyl (more preferably di(C₁-C₄)alkylcarbamoyl, most preferably dimethylcarbamoyl, diethylcarbamoyl or diisopropylcarbamoyl); bis(hydroxy(lower)alkyl)carbamoyl (more preferably bis(hydroxy(C₁-C₄)alkyl)carbamoyl, most preferably bis(hydroxymethyl)carbamoyl); benzyloxycarbonyl; benzoyl; or pyrrolylcarbonyl, imidazolylcarbonyl, pyrazolylcarbonyl, pyridylcarbonyl, pyrimidylcarbonyl, pyrazinylcarbonyl, pyrrolidinylcarbonyl, piperidylcarbonyl, piperazinylcarbonyl, isoxazolylcarbonyl, morpholinylcarbonyl, furylcarbonyl, oxetanylcarbonyl, oxolanylcarbonyl, tetrahydropyranylcarbonyl or dioxanylcarbonyl, each of which may be substituted with one or two substituent(s) selected from a group consisting of hydroxy, lower alkyl (more preferably C₁-C₄ alkyl, most preferably methyl or ethyl), lower alkoxy (more preferably C₁-C₄ alkoxy, most preferably methoxy or ethoxy), carbamoyl, amino and N-oxido,
R³, R⁴ and R⁵ are independently hydrogen; halogen (more preferably chloro); lower alkyl (more preferably C₁-C₄ alkyl, most preferably methyl, isopropyl or isobutyl); cyclo(lower)alkyl (more preferably cyclo(C₃-C₆)alkyl, most preferably cyclopropyl); cyclo(lower)alkyloxy (more preferably cyclo(C₃-C₆)alkyloxy, most preferably cyclobutyloxy); lower alkoxy (more preferably C₁-C₄ alkoxy, most preferably methoxy, ethoxy or isopropoxy) optionally substituted with one, two or three halogen(s) (more preferably fuluoro(s)); or lower alkanoyl (more preferably C₁-C₄ alkanoyl, most preferably propionyl) optionally substituted with one, two or three halogen(s) (more preferably fuluoro(s)), and
R⁶ is hydrogen or lower alkyl (more preferably C₁-C₄ alkyl, most preferably methyl).

More preferred embodiments of the object compound (I) are as follows: 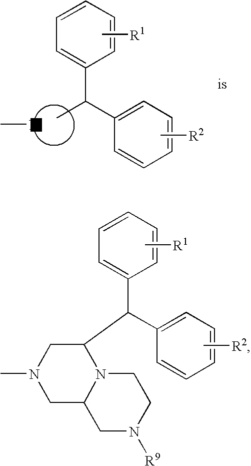
in which
R¹ and R² are each hydrogen,
R⁹ is hydrogen; lower alkanoyl (more preferably C₁-C₄ alkanoyl, most preferably acetyl, propionyl or butyryl) optionally substituted with one, two or three substituent(s) selected from a group consisting of hydroxy, halogen (more preferably fluoro), lower alkoxy (more preferably C₁-C₄ alkoxy, most preferably methoxy), hydroxy(lower)alkyl (more preferably hydroxy-(C₁-C₄)alkyl, most preferably hydroxymethyl), acetylamino, mono(or di)(lower)alkylamino (more preferably mono(or di)(C₁-C₄)alkylamino, most preferably dimethylamino) and pyridyl; di(lower)alkylcarbamoyl (more preferably di(C₁-C₄)alkylcarbamoyl, most preferably dimethylcarbamoyl, diethylcarbamoyl or diisopropylcarbamoyl); bis(hydroxy(lower)alkyl)carbamoyl (more preferably bis(hydroxy(C₁-C₄)alkyl)carbamoyl, most preferably bis(hydroxymethyl)carbamoyl); benzyloxycarbonyl; benzoyl; or pyrrolylcarbonyl, imidazolylcarbonyl, pyrazolylcarbonyl, pyridylcarbonyl, pyrimidylcarbonyl, pyrazinylcarbonyl, pyrrolidinylcarbonyl, piperidylcarbonyl, piperazinylcarbonyl, isoxazolylcarbonyl, morpholinylcarbonyl, furylcarbonyl, oxetanylcarbonyl, oxolanylcarbonyl, tetrahydropyranylcarbonyl or dioxanylcarbonyl, each of which may be substituted with one or two substituent(s) selected from a group consisting of hydroxy, lower alkyl (more preferably C₁-C₄ alkyl, most preferably methyl or ethyl), lower alkoxy (more preferably C₁-C₄ alkoxy, most preferably methoxy or ethoxy), carbamoyl, amino and N-oxido,
R³, R⁴ and R⁵ are independently hydrogen; halogen (more preferably chloro); lower alkyl (more preferably C₁-C₄ alkyl, most preferably methyl, isopropyl or isobutyl); cyclo(lower)alkyl (more preferably cyclo(C₃-C₆)alkyl, most preferably cyclopropyl); cyclo(lower)alkyloxy (more preferably cyclo(C₃-C₆)alkyloxy, most preferably cyclobutyloxy); lower alkoxy (more preferably C₁-C₄ alkoxy, most preferably methoxy, ethoxy or isopropoxy) optionally substituted with one, two or three halogen(s) (more preferably fuluoro(s)); or lower alkanoyl (more preferably C₁-C₄ alkanoyl, most preferably propionyl) optionally substituted with one, two or three halogen(s) (more preferably fuluoro(s)), and
R⁶ is hydrogen.

The Processes 1, 2 and 3 for preparing the object compound (I) of the present invention are explained in detail in the following.

Process 1

The object compound (I) or a salt thereof can be prepared by reacting the compound (II) or its reactive derivative at the imino group or a salt thereof with the compound (III) or a salt thereof.

Suitable reactive derivative at the imino group of the compound (II) may include Schiff's base type imino or its tautomeric enamine type isomer formed by the reaction of the compound (II) with a carbonyl compound such as aldehyde, ketone or the like; a silyl derivative formed by the reaction of the compound (II) with a silyl compound such as bis(trimethylsilyl)acetamide, mono(trimethylsilyl)acetamide, bis(trimethylsilyl)urea or the like; a derivative formed by reaction of the compound (II) with phosphorus trichloride or phosgene and the like.

The reaction is usually carried out in a conventional solvent such as water, alcohol (e.g. methanol, ethanol, etc.), acetone, dioxane, acetonitrile, chloroform, methylene chloride, ethylene chloride, tetrahydrofuran, ethyl acetate, N,N-dimethylformamide, pyridine or any other organic solvent which does not adversely influence the reaction, or the mixture thereof.

The reaction may also be carried out in the presence of a reductive regent such as hydrides (e.g. hydrogen iodide, hydrogen sulfide, lithium aluminum hydride, sodium borohydride, sodium cyanoborohydride, sodium triacetoxyborohydride, etc.), or the like.

The reaction temperature is not critical, and the reaction is usually carried out under cooling to heating.

Process 2

The object compound (Ib) or a salt thereof can be prepared by reacting the compound (Ia) or a salt thereof with the compound (IV) or a salt thereof.

The reaction is usually carried out in a conventional solvent such as water, alcohol (e.g. methanol, ethanol, etc.), acetone, dioxane, acetonitrile, chloroform, methylene chloride, ethylene chloride, tetrahydrofuran, ethyl acetate, N,N-dimethylformamide, pyridine or any other organic solvent which does not adversely influence the reaction. These conventional solvents may also be used in a mixture with water.

The reaction may also be carried out in the presence of an inorganic or organic base such as alkali metal carbonate (e.g. potassium carbonate, etc.), alkali metal bicarbonate, tri(lower)alkylamine, pyridine, N-(lower)alkyl-morpholine, N,N-di(lower)alkylethylamine (e.g. N,N-diisopropylethylamine, etc.), N,N-di(lower)alkylbenzylamine, or the like.

The reaction temperature is not critical, and the reaction is usually carried out under cooling to heating.

Process 3

The object compound (Ic) or a salt thereof can be prepared by cyclizing the compound (V) or its reactive derivative at the imino group or a salt thereof.

This reaction can be carried out in substantially the same manner as in Preparation 7.

The object compound (I) and a pharmaceutically acceptable salt thereof have pharmacological activities such as Tachykinin antagonism, especially Substance P antagonism, Neurokinin A antagonism or Neurokinin B antagonism, and therefore are useful for treating or preventing Tachykinin-mediated diseases, particularly Substance P-mediated diseases, for example, respiratory diseases such as asthma, bronchitis (e.g. chronic bronchitis, acute bronchitis and diffuse panbronchiolitis, etc.), rhinitis, cough, expectoration, and the like; ophthalmic diseases such as conjunctivitis, vernal conjunctivitis, and the like; cutaneous diseases such as contact dermatitis, atopic dermatitis, urticaria, and other eczematoid dermatitis, and the like; inflammatory diseases such as rheumatoid arthritis, osteoarthritis, and the like; pains or aches (e.g. migraine, headache, cluster headache, toothache, cancerous pain, back pain, neuralgia, etc.); and the like.

Further, it is expected that the object compound (I) and a pharmaceutically acceptable salt thereof of the present invention are useful for treating or preventing ophthalmic diseases such as glaucoma, uveitis, and the like; gastrointestinal diseases such as ulcer, ulcerative colitis, irritable bowel syndrome, food allergy, and the like; inflammatory diseases such as nephritis, and the like; circulatory diseases such as hypertension, angina pectoris, cardiac failure, thrombosis, Raynaud's disease, and the like; epilepsy; spastic paralysis; pollakiuria; cystitis; bladder detrusor hyperreflexia; urinary incontinence; Parkinson diseases; dimentia; AIDS related dementia; Alzheimer's diseases; Down's syndrome; Huntington's chorea; carcinoid syndrome; disorders related to immune enhancement or suppression; disorders caused by Helicobacter pylori or another spiral urease-positive gram-negative bacterium; sunburn; angiogenesis or diseases caused by angiogenesis; and the like.

It is furthermore expected that the object compound (I) and a pharmaceutically acceptable salt thereof of the present invention are useful for treating or preventing chronic obstructive pulmonary diseases, particularly chronic pulmonary emphysema; iritis; proliferative vitreoretinopathy; psoriasis; inflammatory intestinal diseases, particularly Crohn's diseases; hepatitis; superficial pain on congelation, burn, herpes zoster or diabetic neuropathy; telalgia attended to hyperlipidemia; postoperative neuroma, particularly of mastectomy; vulvar vestibulitis; hemodialysis-associated itching; lichen planus; laryngopharyngitis; bronchiectasis; coniosis; whooping cough; pulmonary tuberculosis; cystic fibrosis; emesis (e.g., nausea, retching, vomiting, acute emesis, delayed emesis, anticipatory emesis, past operative nausea and vomiting (PONV), acute and/or delayed emesis induced by drugs such as cancer chemotherapeutic agents, etc.); mental diseases, particularly anxiety disorders, stress-related disorders, affective disorders, psychological development disorders and schizophrenia; disorders of the the central nervous system such as anxiety, depression, psychosis and schizophrenia; demyelinating diseases such as multiple sclerosis and amyotrophic lateral sclerosis; attenuation of morphine withdrawal; oedema, such as oedema caused by thermal injury; small cell carcinomas, particularly small cell lung cancer (SCLC); hypersensitivity disorders such as poison ivy; fibrosing and collagen diseases such as scleroderma and eosinophilic fascioliasis; reflex sympathetic dystrophy such as shoulder/hand syndrome; addiction disorders such as alcoholism; stress related somatic disorders; rheumatic diseases such as fibrositis; aggressive behaviour, optionally taking an antipsychotic agent together; mania or hypomania, optionally taking an antipsychotic agent together; symptoms associated with Premenstrual Syndrome (PMS) (PMS is also now referred to as Late Luteal Phase Syndrome (LLS); psychosomatic disoredrs; psychoimmunologic disoredrs; attetion deficit disoredrs (ADD) with or without hyperactivity; and the like.

Furthermore, the object compound (I) and a pharmaceutically acceptable salt thereof of the present invention are Central Nervous System (CNS) penetrant.

For therapeutic purpose, the compound (I) and a pharmaceutically acceptable salt thereof of the present invention can be used in a form of pharmaceutical preparation containing one of said compounds, as an active ingredient, in admixture with a pharmaceutically acceptable carrier such as an organic or inorganic solid or liquid excipient suitable for oral, parenteral, external including topical, internal, intravenous, intramuscular, inhalant, nasal, intraarticular, intraspinal, transtracheal or transocular administration. The pharmaceutical preparations may be solid, semi-solid or solutions such as capsules, tablets, pellets, dragees, powders, granules, suppositories, ointments, creams, lotions, inhalants, injections, cataplasms, gels, tapes, eye drops, solution, syrups, aerosols, suspension, emulsion, or the like. If desired, there may be included in these preparations, auxiliary substances, stabilizing agents, wetting or emulsifying agents, buffers and other commonly used additives.

While the dosage of the compound (I) will vary depending upon the age and condition of a patient, an average single dose of about 0.1 mg, 1 mg, 10 mg, 50 mg, 100 mg, 250 mg, 500 mg and 1000 mg of the compound (I) may be effective for treating Tachykinin-mediated diseases such as asthma and the like. In general, amounts between 0.1 mg/body and about 1,000 mg/body may be administered per day.

In order to show the utility of the object compound (I) and a pharmaceutically acceptable salt thereof, the pharmacological test data of some representative compounds of the present invention is shown in the following.

Emesis in the Dog

[I] Test Method

Individually housed adult female dogs (8 to 15 kg) were given an i.v. injection of a solution containing a test compound. 5 Min later the emetic responses (retching and vomiting) were induced by administration of subcutaneous apomorphine (0.1 mg/0.5 ml/kg) and observed for the next 60 min. The timing and number of retches and vomits observed were recorded for each animal. An individual animal was tested with at least 10 days between experiments.

[II] Test Result

The following Test Compound showed 100% inhibition rate of emesis in the dog at the dose of 1.0 mg/kg.

Test Compound: The Object Compound of the Example 1

The following Preparations and Examples are given for the purpose of illustrating this invention.

Preparation 1

To an ice-cooled solution of tert-butyl (2R)-2-benzhydryl-1-piperazinecarboxylate (214 mg) and triethylamine (0.127 ml) in dichloromethane was added dropwise benzyl chloroformate (0.104 ml) over 10 minutes. After stirring for 10 minutes, the mixture was stirred at room temperature for 2 hours. The solution was washed with aqueous sodium bicarbonate (50 ml), dried over magnesium sulfate, and evaporated under reduced pressure. The residue was purified by column chromatography on silica gel using a mixed solvent of hexane and ethyl acetate (4:1). The fractions containing the objective compound were collected and evaporated under reduced pressure to give colorless oil of 4-benzyl 1-tert-butyl (2R)-2-benzhydryl-1,4-piperazinedicarboxylate (0.246 g).

NMR (CDCl₃, δ): 1.20-1.40 (9H, m), 2.70-3.00 (3H, m), 3.60-4.30 (4H, m), 4.80-5.20 (3H, m), 7.14-7.40 (15H, m)

MASS (API-ES): 509 (M+Na)⁺

Preparation 2

To an ice-cooled solution of 4-benzyl 1-tert-butyl (2R)-2-benzhydryl-1,4-piperazinedicarboxylate in dichloromethane (1 ml) was added dropwise 4N hydrogen chloride in ethyl acetate (10 ml) over 10 minutes. The mixture was stirred at room temperature overnight. The resulting precipitate was triturated with diisopropyl ether, collected by filtration, and washed with diisopropyl ether to give a solid of benzyl (3R)-3-benzhydryl-1-piperazinecarboxylate hydrochloride (0.2 g).

NMR (DMSO-d₆, δ): 2.9-5.20 (10H, m), 7.24-7.54 (15H, m), 8.00-8.30 (1H, m), 9.60-9.90 (1H, m)

MASS (API-ES): 387 (M+H)⁺, 409 (M+Na)⁺(free)

Preparation 3

To an ice-cooled solution of benzyl (3R)-3-benzhydryl-1-piperazinecarboxylate hydrochloride (0.2 g) and 1-methyl-1H-pyrazole-4-carbaldehyde (260 mg) in dichloromethane (10 ml) was added sodium triacetoxyborohydride (802 mg) at room temperature. After stirring overnight, the solution was washed with aqueous sodium bicarbonate, dried over magnesium sulfate, and evaporated under reduced pressure. The residue was purified by column chromatography on silica gel using a mixed solvent of toluene and ethyl acetate (4:1). The fractions containing the objective compound were collected and concentrated under reduced pressure to give a syrup of benzyl (3R)-3-benzhydryl-4-[(1-methyl-1H-pyrazol-4-yl)methyl]-1-piperazinecarboxylate.

MASS (API-ES): 481 (M+H)⁺, 503 (M+Na)⁺

The syrup was dissolved into methanol and the solution was hydrogenated over 10% palladium-charcoal (50% wet, 20 mg) at room temperature under atmospheric pressure for 10 hours. After removal of the catalyst by filtration, the filtrate was evaporated under reduced pressure to give the objective compound as a syrup. The syrup was dissolved into 4N hydrogen chloride in ethyl acetate (10 ml) at room temperature. After stirring for 4 hours, the mixture was concentrated under reduced pressure to give a powder of (2R)-2-benzhydryl-1-[(1-methyl-1H-pyrazol-4-yl)methyl]-piperazine dihydrochloride (160 mg).

NMR (DMSO-d₆, δ): 2.90-5.20 (13H, m), 7.24-7.54 (12H, m), 9.50-10.30 (2H, m)

MASS (API-ES): 347 (M+H)⁺(free)

Preparation 4

The following compound was obtained according to a similar manner to that of Preparation 84.

### N-[(4R, 7R, 8aS)-4-Benzhydryloctahydropyrrolo[1,2-a]pyridin-7-yl]acetamide Dihydrochloride

NMR (DMSO-d₆, δ): 1.71 (3H, s), 2.94-4.45 (13H, m), 7.21-7.52 (10H, m), 8.18 (1H, m), 9.72 (2H, m)

MASS (APCI): 350 (M+H)⁺(free)

Preparation 5

A solution of 3-bromo-1,1-bis(4-fluorophenyl)acetone (1.21 g), 4-benzyl 1-tert-butyl (2R)-2-[[(2-methoxybenzyl)-amino]methyl]-1,4-piperazinedicarboxylate (1.05 g) in tetrahydrofuran (13 ml) and N,N-diisopropylethylamine (0.9 ml) was stirred at room temperature for 2 hours. To the mixture was added 4-benzyl 1-tert-butyl (2R)-2-[[(2-methoxybenzyl)amino]methyl]-1,4-piperazinedicarboxylate (0.42 g) and N,N-diisopropylethylamine (0.9 ml), and the mixture was stirred at room temperature overnight. The mixture was quenched with water (200 ml), added brine, and extracted with ethyl acetate (×3). The combined extracts were washed with water (×2) and brine, dried over magnesium sulfate and evaporated under reduced pressure. The residue was purified by column chromatography on silica gel using a mixed solvent of hexane and ethyl acetate (3:1) to give 4-benzyl 1-tert-butyl (2R)-2-[[N-[3,3-bis(4-fluorophenyl)-2-oxopropyl]-N-(2-methoxybenzyl)amino]methyl]-1,4-piperazinedicarboxylate (1.12 g) as colorless oil.

NMR (CDCl₃, δ): 1.42 (9H, s), 2.69-5.30 (16H, m), 3.75 (3H, s), 6.75-7.35 (17H, m)

MASS (ESI): 714.6 (M+H)⁺

Preparation 6

The following compound was obtained according to a similar manner to that of Preparation 5.

### 4-Benzyl 1-tert-butyl (2R)-2-[[N-[3,3-bis(4-methylphenyl)-2-oxopropyl]-N-(2-methoxybenzyl)amino]methyl]-1,4-piperazinedicarboxylate

NMR (CDCl₃, δ): 1.41 (9H, s), 2.28-2.35 (6H, m), 2.20-5.30 (19H, m), 6.73-7.38 (17H, m)

MASS (ESI): 706.33 (M+H)⁺

Preparation 7

To a solution of 4-benzyl 1-tert-butyl (2R)-2-[[N-[3,3-bis(4-fluorophenyl)-2-oxopropyl]-N-(2-methoxybenzyl)amino]-methyl]-1,4-piperazinedicarboxylate (1.1 g) in dichloromethane (6 ml) was added 4N hydrogen chloride in ethyl acetate (4.6 ml) at 0° C. After stirring for 2 hours at room temperature, the mixture was concentrated under reduced pressure. To the solution of the residue in dichloromethane (10 ml) was added sodium triacetoxyborohydride (980 mg) at 0° C., and the mixture was stirred at room temperature for 2 hours. The mixture was quenched with aqueous saturated sodium bicarbonate and extracred with ethyl acetate (×3). The combined organic layers were washed with brine, dried over magnesium sulfate and evaporated under reduced pressure. The residue was purified by column chromatography on silica gel (50 ml) using a mixed solvent of hexane and ethyl acetate (9:1 to 7:3) to give benzyl (6R, 9aR)-6-[bis(4-fluorophenyl)methyl]-8-(2-methoxybenzyl)octahydro-2H-pyrazino[1,2-a]pyrazine-2-carboxylate (628 mg) as a colorless oil.

NMR (CDCl₃, δ): 1.85-1.99 (3H, m), 2.30-2.50 (2H, m), 2.60-3.00 (4H, m), 3.12-3.20 (1H, m), 3.38 (1H, d, J=13.5 Hz), 3.45 (1H, d, J=13.5 Hz), 3.70 (3H, m), 3.70-3.90 (2H, m), 4.21 (1H, d, J=6.6 Hz), 5.09 (2H, s), 6.77-7.38 (17H, m)

MASS (ESI): 598.8 (M+H)⁺

Preparation 8

The following compound was obtained according to a similar manner to that of Preparation 7.

### Benzyl (6R, 9aR)-6-[bis(4-methylphenyl)methyl]-8-(2-methoxybenzyl)octahydro-2H-pyrazino[1,2-a]pyrazine-2-carboxylate

NMR (CDCl₃, δ): 2.27 (6H, d, J=2.0 Hz), 1.80-2.95 (9H, m), 3.20-3.30 (1H, m), 3.38 (1H, d, J=13.7 Hz), 3.43 (1H, d, J=13.7 Hz), 3.68 (3H, s), 3.68-3.90 (2H, m), 4.10 (1H, d, J=6.9 Hz), 5.07 (2H, s), 6.76-7.38 (17H, m)

MASS (ESI): 591.02 (M+H)⁺

Preparation 9

To a solution of benzyl (6R, 9aR)-6-[bis(4-fluorophenyl)methyl]-8-(2-methoxybenzyl)octahydro-2H-pyrazino[1,2-a]pyrazine-2-carboxylate (608 mg) in 1,2-dichloroethane (6 ml) was added chloroformic acid 1-chloroethyl ester (0.165 ml) at room temperature. After stirring at 80° C. for 40 minutes, the mixture was concentrated under reduced pressure. The residue was dissolved into methanol (6 ml) and stirred for 40 minutes at 75° C. The mixture was concentrated under reduced pressure to give oil. To the oil was added a mixture of methanol (0.5 ml) and diisopropyl ether (30 ml), and the mixture was stirred for 30 minutes. The resulting powder was collected by filtration and dried under reduced pressure to give benzyl (6R, 9aR)-6-[bis(4-fluorophenyl)methyl]octahydro-2H-pyrazino[1,2-a]pyrazine-2-carboxylate dihydrochloride (574 mg) as a powder.

NMR (DMSO-d₆, δ): 2.16-3.84 (12H, m), 4.40 (1H, d, J=8.8 Hz), 5.07 (2H, s)

MASS (ESI): 478.3 (M+H)⁺(free)

Preparation 10

The following compound was obtained according to a similar manner to that of Preparation 9.

### Benzyl (6R, 9aR)-6-[bis(4-methylphenyl)methyl]octahydro-2H-pyrazino[1,2-a]pyrazine-2-carboxylate Dihydrochloride

NMR (DMSO-d₆, δ): 2.24 (6H, br s), 2.15-3.80 (12H, m), 4.24 (1H, d, J=8.5 Hz), 5.06 (2H, s), 7.07-7.35 (13H, m)

MASS (ESI): 470.16 (M+H)⁺(free)

Preparation 11

To a solution of benzyl (6R, 9aR)-6-[bis(4-fluorophenyl)methyl]octahydro-2H-pyrazino[1,2-a]pyrazine-2-carboxylate dihydrochloride (548 mg) in tetrahydrofuran (6 ml) was added triethylamine (0.465 ml) and di-tert-butyl dicarbonate (250 mg), and the mixture was stirred at room temperature for 5 hours. The mixture was quenched with water, and extracted with ethyl acetate (×3). The combined extracts were washed with 1N hydrochloric acid, aqueous saturated sodium bicarbonate and brine, dried over magnesium sulfate and evaporated under reduced pressure. The residue was purified by column chromatography on silica gel (50 ml, ethyl acetate/hexane (1:20 to 4:6)) to give 8-benzyl 2-tert-butyl (4R, 9aS)-4-[bis(4-fluorophenyl)methyl]hexahydro-2H-pyrazino[1,2-a]pyrazine-2,8(1H)-dicarboxylate (354 mg) as an oil.

NMR (CDCl₃, δ): 1.34 (9H, br s), 2.40-3.20 (7H, m), 3.50-3.90 (5H, m), 4.16 (1H, d, J=7.0 Hz), 5.10 (2H, s), 6.92-7.02 (4H, m), 7.18-7.39 (9H, m)

MASS (ESI): 579.3 (M+H)⁺

Preparation 12

The following compound was obtained according to a similar manner to that of Preparation 11.

### 8-Benzyl 2-tert-butyl (4R, 9aS)-4-[bis(4-methylphenyl)-methyl]hexahydro-2H-pyrazino[1,2-a]pyrazine-2,8(1H)-dicarboxylate

NMR (CDCl₃, δ): 1.34 (9H, br s), 2.28 (6H, d, J=2.9 Hz), 1.80-3.10 (7H, m), 3.50-4.00 (5H, m), 4.10 (1H, d, J=6.9 Hz), 5.09 (2H, s), 7.02-7.18 (8H, m), 7.29-7.41 (5H, m)

MASS (ESI): 570.48 (M+H)⁺

Preparation 13

A mixture of 8-benzyl-2-tert-butyl (4R, 9aS)-4-[bis(4-fluorophenyl)methyl]hexahydro-2H-pyrazino[1,2-a]pyrazine-2,8(1H)-dicarboxylate (354 mg) and palladium on carbon (70 mg) in methanol (4 ml) was stirred at room temperature under hydrogen for 2.5 hours. The mixture was filtered and evaporated to give tert-butyl (4R, 9aS)-4-[bis(4-fluorophenyl)methyl]octahydro-2H-pyrazino[1,2-a]pyrazine-2-carboxylate (280 mg) as an oil.

NMR (CDCl₃, δ): 1.34 (9H, br s), 1.90-3.00 (10H, m), 3.50-3.80 (2H, m), 4.20 (1H, d, J=6.6 Hz), 6.92-7.02 (4H, m), 7.19-7.26 (4H, m)

MASS (ESI): 444.7 (M+H)⁺

Preparation 14

The following compound was obtained according to a similar manner to that of Preparation 13.

### tert-Butyl (4R, 9aS)-4-[bis(4-methylphenyl)methyl]-octahydro-2H-pyrazino[1,2-a]pyrazine-2-carboxylate

NMR (CDCl₃, δ): 1.34 (9H, br s), 2.28 (6H, d, J=2.9 Hz), 1.80-3.10 (7H, m), 3.50-4.00 (5H, m), 4.10 (1H, d, J=6.9 Hz), 5.09 (2H, s), 7.02-7.18 (8H, m), 7.29-7.41 (5H, m)

MASS (ESI): 570.48 (M+H)⁺

Preparation 15

To a mixture of tert-butyl (4R, 9aS)-4-[bis(4-fluorophenyl)methyl]octahydro-2H-pyrazino[1,2-a]pyrazine-2-carboxylate (279.5 mg), hydroxyacetic acid (120 mg), and 1-hydroxybenzotriazole hydrate (93.7 mg) in dichloromethane (1 ml) was added 1-ethyl-3-(3-dimethylaminopropyl)carbodiimide hydrochloride (242 mg) at room temperature. After stirring at room temperature overnight, the mixture was quenched with aqueous saturated sodium bicarbonate and extracted with ethyl acetate (×3). The combined extracts were washed with brine, dried over magnesium sulfate and evaporated under reduced pressure. The residue was dissolved into a mixture of methanol (12 ml) and 1N sodium hydroxide solution (4 ml), then the mixture was stirred for 3.5 hours. The mixture was quenched with aqueous saturated ammonium chloride and extracted with ethyl acetate (×3). The combined extracts were dried over magnesium sulfate and evaporated under reduced pressure. The residue was purified with preparative TLC (1 mm×2, methanol/chloroform=1/9) to give tert-butyl (4R, 9aS)-4-[bis(4-fluorophenyl)methyl]-8-glycoloyloctahydro-2H-pyrazino[1,2-a]pyrazine-2-carboxylate (204 mg) as an oil.

NMR (CDCl₃, δ): 1.29 (9H, br s), 2.00-3.90 (12H, m), 4.00-4.30 (3H, m), 6.93-7.03 (4H, m), 7.18-7.26 (4H, m)

MASS (ESI): 502.19 (M+H)⁺

Preparation 16

The following compound was obtained according to a similar manner to that of Preparation 15.

### tert-Butyl (4R, 9aS)-4-[bis(4-methylphenyl)methyl]-8-glycoloyloctahydro-2H-pyrazino[1,2-a]pyrazine-2-carboxylate

NMR (CDCl₃, δ): 1.34 (9H, br s), 2.28 (6H, d, J=2.7 Hz), 1.90-4.30 (15H, m), 7.03-7.18 (8H, m)

MASS (ESI): 494.2 (M+H)⁺

Preparation 17

tert-Butyl (4R, 9aS)-4-[bis(4-fluorophenyl)methyl]-8-glycoloyloctahydro-2H-pyrazino[1,2-a]pyrazine-2-carboxylate (200 mg) was dissolved in a solution of hydrogen chloride in ethyl acetate (4N, 2 ml) and stirred at room temperature for 30 minutes. The mixture was quenched with aqueous saturated sodium bicarbonate and extracted with ethyl acetate (×3). The combined extracts were washed with brine, dried over magnesium sulfate and evaporated under reduced pressure to give 2-[(6R, 9aR)-6-[bis(4-fluorophenyl)methyl]octahydro-2H-pyrazino[1,2-a]pyrazin-2-yl]-2-oxoethanol (149 mg) as an oil.

NMR (CDCl₃, δ): 1.90-3.20 (12H, m), 4.07-4.25 (3H, m), 6.90-7.04 (4H, m), 7.15-7.26 (4H, m)

MASS (ESI): 402.23 (M+H)⁺

Preparation 18

The following compound was obtained according to a similar manner to that of Preparation 17.

### 2-[(6R, 9aR)-6-[Bis(4-methylphenyl)methyl]octahydro-2H-pyrazino[1,2-a]pyrazin-2-yl]-2-oxoethanol

NMR (CDCl₃, δ): 2.29 (6H, d, J=3.8 Hz), 1.80-3.30 (12H, m), 4.06-4.22 (3H, m), 7.01-7.16 (8H, m)

MASS (ESI): 394.26 (M+H)⁺

Preparation 19

To a solution of isopropylamine (8.62 ml) in tetrahydrofuran (100 ml) was added dropwise butyllithium (1.45 M in hexane, 38.6 ml) at −78° C. over 5 minutes. After stirred at −78° C. for 10 minutes, the mixture was added to a solution of ethyl bis(4-methylphenyl)acetate (2.0 g) and chloroiodomethane (3.26 ml) in tetrahydrofuran (36 ml) at −60˜70° C. over 30 minutes. The mixture was stirred at −75° C. for 2.5 hours, and then added dropwise a solution of acetic acid (15 ml) in tetrahydrofuran (30 ml) over 15 minutes. After stirred for 10 minutes at −78° C., the solution was poured into a mixture of water and ethyl acetate. The aqueous layer was separated and extracted with ethyl acetate (×2). The combined organic layers were washed with saturated sodium bicarbonate (×2) and brine, dried over sodium sulfate, filtered and evaporated to give brown oil. The oil was purified by column chromatography (silica gel 200 ml), eluated with a mixture of ethyl acetate and hexane (2:98, 500 ml) to give 3-chloro-1,1-bis(4-methylphenyl)acetone (1.805 g) as an oil.

NMR (CDCl₃, δ): 2.31 (6H, br s), 4.09-4.25 (2H, m), 5.33 (1H, s), 7.05-7.36 (8H, m)

MASS (ESI−): 271.3 (M−H)⁻

Preparation 20

Under nitrogen atmosphere, to a solution of 2,4,6-trimethoxy-5-pyrimidinecarbaldehyde (303 mg) in dichloromethane (5 ml) was added 2 M boron tribromide in dichloromethane (0.96 ml) under iced-cooling. After it was stirred at room temperature for 10 minutes, 2 M boron tribromide in dichloromethane (0.80 ml) was added again to the solution at the same temperature, and then the whole was stirred for 30 minutes. The reaction mixture was poured into ice-water, and the pH of the aqueous layer was adjusted to 4 with aqueous sodium bicarbonate. The organic layer was separated, dried over sodium sulfate, and concentrated under reduced pressure. The resulting residue was purified by column chromatography on silica gel (10 g) using a mixed solvent of dichloromethane and methanol (10:1). The fractions containing the objective compound were collected and evaporated under reduced pressure to give 4-hydroxy-2,6-dimethoxy-5-pyrimidinecarbaldehyde (258 mg) as a light yellow solid.

IR (KBr): 2960, 2837, 2756, 1684, 1647, 1591, 1560, 1508, 1335, 1234 cm⁻¹

NMR (CDCl₃, δ): 4.07 (3H, s), 4.10 (3H, s), 10.04 (1H, s), 13.10 (1H, br)

MASS (API-ES): 207 (M+Na)⁺

Preparation 21

The following compound was obtained according to a similar manner to that of Preparation 20.

### 2-Ethoxy-4-hydroxy-6-methoxy-5-pyrimidinecarbaldehyde

NMR (CDCl₃, δ): 1.51 (3H, t, J=7.1 Hz), 4.09 (3H, s), 4.52 (2H, q, J=7.1 Hz), 10.04 (1H, s), 13.09 (1H, br)

MASS (API-ES): 199 (M+H)⁺

Preparation 22

To a solution of 4-hydroxy-2,6-dimethoxy-5-pyrimidinecarbaldehyde (90 mg) in N,N-dimethylformamide (2 ml) were added 2-iodopropane (249 mg) and cesium fluoride (223 mg) at room temperature. The mixed solution was stirred at 40° C. for 14 hours. After being cooled to room temperature, the mixture was poured into water. The resulting mixture was extracted with ethyl acetate. The extract was washed with brine, dried over sodium sulfate and evaporated under reduced pressure. The residue was purified by preparative TLC eluting with a mixture of hexane and ethyl acetate (4:1) to give 4-isopropoxy-2,6-dimethoxy-5-pyrimidinecarbaldehyde (30 mg) as a colorless powder.

IR (KBr): 2991, 2956, 1691, 1591, 1558, 1487, 1371, 1234, 1103 cm⁻¹

NMR (CDCl₃, δ): 1.41 (6H, d, J=6.2 Hz), 4.03 (3H, s), 4.08 (3H, s), 5.49 (1H, m), 10.20 (1H, m)

MASS (API-ES): 249 (M+Na)⁺

Preparation 23

The following compounds were obtained according to a similar manner to that of Preparation 22.

### (1) 4-Ethoxy-2,6-dimethoxy-5-pyrimidinecarbaldehyde

IR (KBr): 2993, 2881, 1687, 1593, 1558, 1377, 1232, 1151 cm⁻¹

NMR (CDCl₃, δ): 1.44 (3H, t, J=7.1 Hz), 4.03 (3H, s), 4.08 (3H, s), 4.55 (2H, q, J=7.1 Hz), 10.22 (1H, s)

MASS (API-ES): 235 (M+Na)⁺

### (2) 2-Isopropoxy-4,6-dimethoxypyrimidine

NMR (CDCl₃, δ): 1.39 (6H, d, J=6.3 Hz), 3.94 (6H, s), 5.27 (1H, m), 5.68 (1H, s)

MASS (API-ES): 221 (M+Na)⁺

### (3) 2-Ethoxy-4,6-dimethoxypyrimidine

IR (KBr): 2985, 2956, 1585, 1194, 1159, 1099 cm⁻¹

NMR (CDCl₃, δ): 1.42 (3H, t, J=7.1 Hz), 3.92 (6H, s), 4.40 (2H, q, J=7.1 Hz), 5.70 (1H, s)

MASS (API-ES): 207 (M+Na)⁺

### (4) 2,4-Diethoxy-6-methoxy-5-pyrimidinecarbaldehyde

NMR (CDCl₃, δ): 1.40-1.49 (6H, m), 4.07 (3H, s), 4.41-4.60 (4H, m), 10.21 (1H, s)

MASS (API-ES): 227 (M+H)⁺

### (5) 2-Ethoxy-4-isopropoxy-6-methoxy-5-pyrimidinecarbaldehyde

mp: 53-54° C.

NMR (CDCl₃, δ): 1.40 (6H, d, J=6.3 Hz), 1.44 (3H, t, J=7.1 Hz), 4.09 (3H, s), 4.46 (1H, q, J=7.1 Hz), 5.48 (1H, sept, J=6.3 Hz), 10.20 (1H, s)

### (6) 4-(Cyclobutyloxy)-2,6-dimethoxypyrimidine

NMR (CDCl₃, δ): 1.60-1.92 (2H, m), 2.04-2.21 (2H, m), 2.37-2.52 (2H, m), 3.92 (3H, s), 3.95 (3H, s), 5.11 (1H, quintet, J=7.2 Hz), 5.64 (1H, s)

MASS (ESI): 211.22 (M+H)⁺

### (7) 4,6-Diethoxy-2-methoxypyrimidine

mp: 52° C.

IR (KBr): 1599, 1568, 1439, 1367, 1331, 1176 cm⁻¹

NMR (CDCl₃, δ): 1.37 (6H, t, J=7.1 Hz), 3.95 (3H, s), 4.34 (4H, q, J=7.1 Hz), 5.66 (1H, s)

MASS (ES): 221.3 (M+Na)⁺, 199.3 (M+H)⁺

Preparation 24

To a solution of 4,6-dimethoxy-2-pyrimidinamine (1.04 g) in acetic acid (10 ml) was added portionwise sodium nitrite (925 mg) with maintaining reaction temperature below 30° C., and then the mixture was stirred at room temperature for 15 hours. After the reaction mixture was concentrated under reduced pressure, dichloromethane and water were added to the residue with sitrring, and the pH of the aqueous layer was adjusted to 3 with saturated aqueous sodium bicarbonate. The organic layer was separated, dried over sodium sulfate, and concentrated under reduced pressure. The resulting residue was purified by column chromatography on silica gel (30 g) using a mixed solvent of dichloromethane and methanol (15:1). The fractions containing the objective compound were collected and evaporated under reduced pressure to give 4,6-dimethoxy-2(1H)-pyrimidinone (800 mg) as a gray powder.

IR (KBr): 3386, 2850, 1666, 1628, 1574, 1460, 1342, 1227 cm⁻¹

NMR (DMSO-d₆, δ): 3.81 (6H, s), 5.49 (1H, s), 11.64 (1H, br)

Preparation 25

The following compound was obtained according to a similar manner to that of Preparation 24.

### 2,6-Dimethoxy-4-pyrimidinol

NMR (DMSO-d₆, δ): 3.77 (3H, s), 3.89 (3H, s), 5.25 (1H, s), 12.03 (1H, br)

MASS (API-ES): 157 (M+H)⁺

Preparation 26

To an ice-cooled solution of 2-isopropoxy-4,6-dimethoxypyrimidine (370 mg) in N,N-dimethylformamide (3 ml) was added dropwise phosphorus oxychloride (458 mg) under nitrogen atmosphere. The mixed solution was stirred at room temperature for 2 days. The reaction mixture was quenched with water (5 ml), and the whole was extracted with ethyl acetate, the extract was washed with brine, dried over sodium sulfate, and concentrated under reduced pressure. The resulting residue was purified by column chromatography on silica gel (10 g) using a mixed solvent of hexane and ethyl acetate (2:1). The fractions containing the objective compound were collected and evaporated under reduced pressure to give 2-isopropoxy-4,6-dimethoxy-5-pyrimidinecarbaldehyde (350 mg) as a white powder.

IR (KBr): 2974, 2870, 1691, 1595, 1475, 1427, 1375, 1236, 1136 cm⁻¹

NMR (CDCl₃, δ): 1.43 (6H, d, J=6.1 Hz), 4.07 (6H, S), 5.35 (1H, m), 10.20 (1H, s)

MASS (API-ES): 249 (M+Na)⁺

Preparation 27

The following compounds were obtained according to a similar manner to that of Preparation 26.

### (1) 2-Ethoxy-4,6-dimethoxy-5-pyrimidinecarbaldehyde

IR (KBr): 2995, 2960, 2885, 1685, 1577, 1568, 1365, 1223, 1132 cm⁻¹

NMR (CDCl₃, δ): 1.46 (3H, t, J=7.1 Hz), 4.08 (6H, s), 4.49 (2H, q, J=7.1 Hz), 10.21 (1H, s)

MASS (API-ES): 235 (M+Na)⁺

### (2) 4-(Cyclobutyloxy)-2,6-dimethoxy-5-pyrimidinecarbaldehyde

NMR (CDCl₃, δ): 1.40-2.00 (2H, m), 2.15-2.35 (2H, m), 2.40-2.58 (2H, m), 4.03 (3H, s), 4.07 (3H, s), 5.32 (1H, quintet, J=7.7 Hz), 10.22 (1H, s)

MASS (ESI): 239 (M+H)⁺

### (3) 4-(2,2-Difluoroethoxy)-2,6-dimethoxy-5-pyrimidinecarbaldehyde

IR (KBr): 2964, 1685, 1599, 1560, 1377, 1236, 1165 cm⁻¹

NMR (CDCl₃, δ): 4.05 (3H, s), 4.11 (3H, s), 4.68 (2H, dt, J=4.1, 13 Hz), 6.15 (1H, tt, J=4.1, 55 Hz), 10.22 (1H, s)

MASS (API-ES): 249 (M+H)⁺

### (4) 4,6-Diethoxy-5-formyl-2-methoxypyrimidine

mp: 71° C.

IR (KBr): 1695, 1581, 1481, 1375, 1340, 1230, 1149 cm⁻¹

NMR (CDCl₃, δ): 1.44 (6H, t, J=7.1 Hz), 4.01 (3H, s), 4.55 (4H, q, J=7.1 Hz), 10.22 (1H, s)

MASS (ES): 249.3 (M+Na)⁺, 227.3 (M+H)⁺

Preparation 28

Under nitrogen atmosphere, to a solution of diisopropylamine (169 mg) in tetrahydrofuran (15 ml) was added dropwise 1.56 M butyllithium in hexane (1.03 ml) over 10 minutes below −50° C., and the mixture was stirred at 0° C. for 15 minutes. A solution of 2,4,6-triethoxypyrimidine (273 mg) in tetrahydrofuran (2 ml) was added dropwise to the mixture over 15 minutes below −50° C., and the whole was stirred at the same temperature for 90 minutes. Isopropyl formate (227 mg) was added to the mixture rapidly, and the whole was stirred below −25° C. for 1 hour. The reaction was quenched with 1N hydrochloric acid (3.3 ml), and the whole was stirred under ice-cooling for 10 minutes. The mixture was extracted with ethyl acetate, and the extract was dried over sodium sulfate, and concentrated under reduced pressure. The resulting residue was purified by column chromatography on silica gel (8 g) using a mixed solvent of hexane and ethyl acetate (2:1). The fractions containing the objective compound were collected and evaporated under reduced pressure to give 2,4,6-triethoxy-5-pyrimidinecarbaldehyde (64 mg) as a white powder.

NMR (CDCl₃, δ): 1.31-1.49 (9H, m), 4.35-4.59 (6H, m), 10.22 (1H, s)

MASS (APCI): 241 (M+H)⁺

Preparation 29

The following compound was obtained according to a similar manner to that of Preparation 28.

### 2-Chloro-4,6-dimethoxy-5-pyrimidinecarbaldehyde

IR (KBr): 2964, 2881, 1695, 1554, 1261, 1122 cm⁻¹

NMR (CDCl₃, δ): 4.12 (6H, s), 10.28 (1H, s)

MASS (API-ES): 225 (M+Na)⁺

Preparation 30

Under nitrogen atmosphere, to a solution of 2-chloro-4,6-dimethoxypyrimidine (500 mg) in tetrahydrofuran (9 ml) were added tetrakis(triphenylphosphine)palladium(0) (232 mg) and 0.98 M trimethylaluminum in hexane (3.51 ml) successively at room temperature, and then the whole mixture was refluxed with stirring for 5 hours. After cooling with ice, it was quenched with water. The mixture was partitioned between ethyl acetate and water. The organic phase was separated, washed with brine, dried over magnesium sulfate, and concentrated under reduced pressure. The residue was purified by column chromatography on silica gel (10 g) using a mixed solvent of hexane and ethyl acetate (10:1). The fractions containing the objective compound were collected and evaporated under reduced pressure to give 4,6-dimethoxy-2-methylpyrimidine (320 mg) as a colorless powder.

IR (KBr): 2958, 1589, 1377, 1252, 1161 cm⁻¹

NMR (CDCl₃, δ): 2.52 (3H, s), 3.93 (6H, s), 5.85 (1H, s)

MASS (APCI): 155 (M+H)

Preparation 31

To a solution of 4,6-dimethoxy-2-methylpyrimidine (312 mg) in acetic acid (3 ml) was added N-bromosuccinimide (414 mg) at room temperature. After the reaction mixture was heated at 70° C. with stirring for 1 hour, it was concentrated under reduced pressure. The resulting residue was partitioned between dichloromethane and water. The pH of the aqueous layer was adjusted to 7 with aqueous sodium bicarbonate. The organic phase was separated, washed with brine, dried over sodium sulfate, and concentrated under reduced pressure. The resulting residue was purified by column chromatography on silica gel (15 g) using a mixed solvent of hexane and ethyl acetate (4:1). The fractions containing the objective compound were collected and evaporated under reduced pressure to give 5-bromo-4,6-dimethoxy-2-methylpyrimidine (370 mg) as a white solid.

IR (KBr): 3003, 2958, 1568, 1356, 1124 cm⁻¹

NMR (CDCl₃, δ): 2.50 (3H, s), 4.01 (6H, s)

Preparation 32

The following compound was obtained according to a similar manner to that of Preparation 31.

### 5-Bromo-4-isobutyl-2,6-dimethoxypyrimidine

NMR (CDCl₃, δ): 0.96 (6H, d, J=6.68 Hz), 2.17-2.31 (1H, m), 2.71 (2H, d, J=7.10 Hz), 3.94 (3H, s), 4.00 (3H, s)

MASS (API-ES): 277 (M+H)⁺

Preparation 33

Under nitrogen atmosphere, to a solution of 5-bromo-4,6-dimethoxy-2-methylpyrimidine (232 mg) in diethyl ether (10 ml) was added dropwise 1.56 M butyllithium in hexane (0.72 ml) over 10 minutes below −70° C. After the mixture was stirred at the same temperature for 60 minutes, isopropyl formate (219 mg) was added to the mixture at one portion, and the whole was stirred at the same temperature for 20 minutes. The reaction was quenched with 1N hydrochloric acid, and the whole was stirred under ice-cooling for 10 minutes. The resulting mixture was extracted with ethyl acetate, and the extract was dried over sodium sulfate, and concentrated under reduced pressure. The resulting residue was purified by column chromatography on silica gel (10 g) using a mixed solvent of hexane and ethyl acetate (6:1). The fractions containing the objective compound were collected and evaporated under reduced pressure to give 4,6-dimethoxy-2-methyl-5-pyrimidinecarbaldehyde (120 mg) as a white solid.

IR (KBr): 2962, 2866, 1687, 1566, 1556, 1126 cm⁻¹

NMR (CDCl₃, δ): 2.55 (3H, s), 4.07 (6H, s), 10.32 (1H, s)

MASS (API-ES): 205 (M+Na)⁺

Preparation 34

The following compound was obtained according to a similar manner to that of Preparation 33.

### 4-Isobutyl-2,6-dimethoxy-5-pyrimidinecarbaldehyde

NMR (CDCl₃, δ): 0.94 (6H, d, J=6.70Hz), 2.08-2.21 (1H, m), 2.98 (2H, d, J=7.06), 4.05 (3H, s), 4.08 (3H, s), 10.35 (1H, S)

MASS (API-ES): 225 (M+H)⁺

Preparation 35

To a suspension of sodium hydride (2.61 g, in mineral oil) in xylene (30 ml) was added dropwise a solution of cyclobutanol (4.56 g) in xylene (40 ml) at 0° C. over 5 minutes, and the mixture was stirred at room temperature for 15 minutes. To the mixture was added a solution of 4-chloro-6-methoxy-2-pyrimidinamine (10.1 g) in xylene (40 ml), and the mixture was stirred at 150° C. for 3 hours. After cooled to room temperature, the mixture was poured into a mixture of water (400 ml) and ethyl acetate (400 ml). The aqueous layer was separated and extracted with ethyl acetate (200 ml) and then dichloromethane (200 ml). The combined organic layer were dried over magnesium sulfate, filtered and evaporated under reduced pressure to give 16 g of crude oil. The oil was purified by column chromatography (silica gel 1000 ml), eluted with a mixture of ethyl acetate and hexane (5:95 to 20:80) to give 4-(cyclobutyloxy)-6-methoxy-2-pyrimidinamine (7.04 g) as an oil.

NMR (CDCl₃, δ): 1.50-1.90 (2H, m), 2.04-2.28 (2H, m), 2.32-2.49 (2H, m), 3.84 (3H, s), 4.97 (1H, quintet, J=7.3 Hz), 5.38 (1H, s)

MASS (ESI): 196.3 (M+H)⁺

Preparation 36

To a solution of 4-(cyclobutyloxy)-6-methoxy-2-pyrimidinamine (6.758 g) in acetic acid (68 ml) was added portionwise sodium nitrite (4.78 g) at room temperature, and the mixture was stirred at room temperature overnight. Then the mixture was evaporated at 40° C. for 30 minutes. Water (300 ml) was added to the residue, and the mixture was extracted with a mixture of methanol and dichloromethane (1:10, 300 ml, 250 ml×2). The combined organic layers were washed with brine, dried over magnesium sulfate, filtered and evaporated under reduced pressure to give 6.3 g of solid. The solid was purified by column chromatography (silica gel 400 ml), eluted with a mixture of methanol and dichloromethane (3:97, 2000 ml) to give 4-(cyclobutyloxy)-6-methoxy-2-pyrimidinol (5.03 g) as a solid.

NMR (CDCl₃, δ): 1.55-1.95 (2H, m), 2.10-2.50 (4H, m), 3.92 (3H, s), 4.89 (1H, quintet, J=7.2 Hz), 5.13 (1H, s)

Preparation 37

To a solution of 2,4,6-trichloropyrimidine (184 mg) in tetrahydrofuran (2 ml) was added 2 M solution of isobutylmagnesium bromide in ethyl ether (2 ml) at −78° C. and the mixture was allowed to warm to room temperature over 2 hours and then stirred at room temperature for 18 hours. The reaction was quenched with 1N hydrochloric acid and the mixture was extracted twice with ethyl acetates. The combined extracts were washed with saturated aqueous sodium bicarbonate and brine, dried over magnesium sulfate, and evaporated under reduced pressure. The residue was purified by column chromatography on silica gel eluting with ethyl acetate-hexane (1:30) to give 2,4-dichloro-6-isobutylpyrimidine (59 mg).

NMR (CDCl₃, δ): 0.96 (6H, d, J=6.57 Hz), 2.08-2.22 (1H, m), 2.61 (2H, d, J=70.10 Hz)

MASS (API-ES): 227 (M+Na)⁺

Preparation 38

To a solution of 2,4-dichloro-6-isobutylpyrimidine (56 mg) in methanol (1 ml) was added 28% sodium methoxide in methanol (148 mg) and the mixture was heated under refluxing for 0.5 hour. The mixture was partitioned between ethyl acetate and 1N hydrochloric acid. The aqueous phase was neutralized with sodium bicarbonate and extracted twice with ethyl acetates. The combined organic phase was washed with saturated aqueous sodium bicarbonate and brine, dried over magnesium sulfate, and evaporated under reduced pressure to give 4-isobutyl-2,6-dimethoxypyrimidine (42.7 mg).

NMR (CDCl₃, δ): 0.95 (6H, d, J=6.68 Hz), 2.06-2.19 (1H, m), 2.45 (2H, d, J=7.14 Hz), 3.94 (3H, s), 3.97 (3H, s)

MASS (API-ES): 197 (M+Na)⁺

Preparation 39

To a solution of 4-hydroxy-2,6-dimethoxypyrimidine (374 mg) in N,N-dimethylformamide (5 ml) were added 2-bromo-1,1-difluoroethane (1.04 g), cesium fluoride (1.09 g) and potassium iodide (79.5 mg) at room temperature. The mixed solution was stirred at 40° C. for 14 hours. After being cooled to room temperature, the mixture was poured into water. The resulting mixture was extracted with ethyl acetate. The extract was washed with brine, dried over sodium sulfate and evaporated under reduced pressure. The residue was purified by column chromatography on silica gel (10 g) using a mixed solvent of hexane and ethyl acetate (1:1). The fractions containing the objective compound were collected and evaporated under reduced pressure to give 4-(2,2-difluoroethoxy)-2,6-dimethoxypyrimidine (165 mg) as a colorless solid.

IR (KBr): 3006, 2958, 1591, 1367, 1196, 1065 cm⁻¹

NMR (CDCl₃, δ): 3.94 (3H, s), 3.97 (3H, s), 4.54 (2H, dt, J=4.2, 13 Hz), 5.80 (1H, s), 6.08 (1H, tt, J=4.2, 55 Hz)

MASS (API-ES): 221 (M+H)⁺

Preparation 40

The following compound was obtained according to a similar manner to that of Preparation 39.

### 2-Amino-4,6-diethoxypyrimidine

mp: 100-101° C.

IR (KBr): 3292, 3346, 3222, 1653, 1567, 1425, 1387, 1346, 1171, 1039 cm⁻¹

NMR (CDCl₃, δ): 1.35 (6H, t, J=7.0 Hz), 4.23 (4H, q, J=7.0 Hz), 4.79 (2H, br s), 5.42 (1H, s)

MASS (ES): 184.3 (M+H)⁺

Preparation 41

The following compound was obtained according to a similar manner to that of Preparation 36.

### 4,6-Diethoxy-2-hydroxypyrimidine

mp: 187-190° C.

IR (KBr): 1635, 1568, 1429, 1327, 1196 cm⁻¹

NMR (CDCl₃, δ): 1.40 (6H, t, J=7.1 Hz), 4.28 (4H, q, J=7.1 Hz), 5.21 (1H, s), 12.05 (1H, br s)

Preparation 42

To a solution of diisopropylamine (1.77 g) in tetrahydrofuran (43 ml) was added dropwise 1.56 M solution of butyllithium in hexane (10.2 ml) below −65° C. and the mixture was stirred at −78° C. for 20 minutes. To this solution was added dropwise 2,4,6-trimethoxypyrimidine (0.9 g) in tetrahydrofuran (10 ml) at −78° C. and the resulting mixture was stirred at the same temperature for 20 minutes. Acetaldehyde (2.33 g) was added and the reaction mixture was allowed to warm to room temperature over 1 hour. The reaction was quenched with water and the resulting mixture was extracted twice with ethyl acetates. The combined extracts were washed with brine, dried over magnesium sulfate, and concentrated under reduced pressure. The residue was purified by column chromatography on silica gel eluting with ethyl acetate-hexane (1:4-1:2) to give 1-(2,4,6-trimethoxy-5-pyrimidinyl)ethanol (840 mg).

NMR (CDCl₃, δ): 1.46 (3H, d, J=6.66 Hz), 2.95 (1H, d, J=10.98 Hz), 3.96 (3H, s), 3.98 (6H, s), 5.01-5.10 (1H, m)

MASS (API-ES): 237 (M+Na)⁺

Preparation 43

To a solution of 1-(2,4,6-trimethoxy-5-pyrimidinyl)-ethanol (21.4 mg) in dichloromethane (0.5 ml) was added thionyl chloride (35.7 mg) at 0° C. and the mixture was stirred at the same temperature for 0.5 hour. The reaction was quenched with saturated aqueous sodium bicarbonate and the resulting mixture was extracted twice with ethyl acetate. The combined extracts were washed with brine, dried over magnesium sulfate, and evaporated under reduced pressure to give 5-(1-chloroethyl)-2,4,6-trimethoxypyridine (18 mg).

NMR (CDCl₃, δ): 1.86 (3H, d, J=7.04 Hz), 3.97 (3H, s), 3.99 (6H, s), 5.53 (1H, q, J=7.02 Hz)

Preparation 44

A mixture of 2-isopropyl-4,6-pyrimidinediol (1.04 g) and phosphoros oxychloride (6.04 ml) was stirred under reflux for 2 hours. After removal of phosphorus oxychloride by evaporation, the resulting residue was partitioned between water and dichloromethane. The organic layer was separated, dried over magnesium sulfate and evaporated under reduced pressure to give an oil of 2-isopropyl-4,6-dichloropyrimidine. To an ice-cooled solution of 2-isopropyl-4,6-dichloropyrimidine in methanol (10 ml) was added dropwise slowly 28% sodium methoxide in methanol over 45 minutes. After stirring for 10 minutes, the mixture was stirred under reflux for 2 hours. After cooling to room temperature, the insoluble materials were filtered off. The filtrate was concentrated under reduced pressure to give a white solid. The solid was partitioned between 1N hydrochloric acid and dichloromethane. The organic layer was separated, dried over magnesium sulfate and evaporated. The residue was purified by column chromatography on silica gel using a mixed solvent of hexane and ethyl acetate (9:1). The fractions containing the objective compound were collected and concentrated under reduced pressure to give 2-iospropyl-4,6-dimethoxypyrimidine (0.55 g) as a syrup.

NMR (CDCl₃, δ): 1.29 (6H, d, J=6.9 Hz), 2.96 (1H, sept, J=6.9 Hz), 3.92 (6H, s), 5.84 (1H, s)

MASS (API-ES): 183 (M+H)⁺

Preparation 45

The following compound was obtained according to a similar manner to that of Preparation 44.

### 2-Cyclopropyl-4,6-dimethoxypyrimidine

NMR (CDCl₃, δ): 0.93-1.01 (2H, m), 1.09-1.16 (2H, m), 2.02-2.10 (1H, m), 3.89 (6H, s), 5.79 (1H, s)

MASS (API-ES): 181 (M+H)⁺

Preparation 46

To a dry ice-methanol cooled suspension of 2-isopropyl-4,6-dimethoxypyrimidine (100 mg) in tetrahydrofuran (2 ml) was added 1.5 M butyllithium in hexane dropwise over 10 minutes between −30° C. and −40° C. The whole was stirred at the same temperature for 30 minutes followed by at between −20° C. and −10° C. for 30 minutes. To the solution was added N,N-dimethylformamide (85 μl) at one time, and then the whole was stirred at 10° C. for 10 minutes. To the solution was added 1N hydrochloric acid (2 ml) (pH ca. 5-6) and the whole was extracted with ethyl acetate (100 ml×3). The combined extract was dried over magnesium sulfate, and evaporated under reduced pressure. The residue was purified by column chromatography on silica gel using a mixed solvent of hexane and ethyl acetate (4:1). The fractions containing the objective compound were collected and concentrated under reduced pressure to give crystals of 2-isopropyl-4,6-dimethoxy-5-pyrimidinecarbaldehyde (54 mg).

mp: 92-93° C.

NMR (CDCl₃, δ): 1.28 (6H, d, J=6.9 Hz), 3.00 (1H, sept, J=6.9 Hz), 4.08 (6H, s), 10.32 (1H, s)

MASS (API-ES): 211 (M+H)⁺

Preparation 47

The following compound was obtained according to a similar manner to that of Preparation 46.

### 2-Cyclopropyl-4,6-dimethoxy-5-pyrimidinecarbaldehyde

NMR (CDCl₃, δ): 1.07-1.15 (2H, m), 1.19-1.27 (2H, m), 2.04-2.14 (1H, m), 4.04 (3H, s), 10.27 (1H, s)

MASS (API-ES): 209 (M+H)⁺

Preparation 48

To an ice-cooling solution of 2-cyclopropyl-4,6-dimethoxy-5-pyrimidinecarbaldehyde (0.297 g) in dichloromethane (3 ml) was added 1 M boron tribromide in dichloromethane dropwise over 30 minutes. The mixture was further stirred at room temperature for 30 minutes and poured into a mixture of ice and water (20 ml). The pH of the mixture was adjusted to 6 with aqueous sodium bicarbonate and the whole was extracted with ethyl acetate. The extract was dried over magnesium sulfate and evaporated under reduced pressure. The residue was dissolved into N,N-dimethylformamide (2 ml) and thereto cesium fluoride (650 mg) and 2-iodopropane (0.42 ml), and the whole was stirred at 42° C. for 10 hours. The reaction mixture was poured into water and extracted with ethyl acetate. The extract was washed with brine, dried over magnesium sulfate and evaporated under reduced pressure. The residue was purified by column chromatography on silica gel using a mixed solvent of hexane and ethyl acetate (10:1). The fractions containing the objective compound were collected and concentrated under reduced pressure to give crystals of 2-cyclopropyl-4-isopropoxy-6-methoxy-5-pyrimidinecarbaldehyde (100 mg).

mp: 46-47° C.

NMR (CDCl₃, δ): 1.03-1.36 (4H, m), 1.38 (6H, d, J=6.2 Hz), 2.03-2.11 (1H, m), 4.04 (3H, s), 5.47 (1H, sept, J=6.2 Hz), 10.26 (1H, s)

MASS (API-ES): 237 (M+H)⁺

Preparation 49

A mixture of 2-cyclopropyl-4,6-pyrimidinediol (0.9 g) and phosphorus oxychloride (5.29 ml) was stirred under reflux for 2 hours. After removal of phosphorus oxychloride by evaporation, the resulting residue was partitioned between water and dichloromethane. The organic layer was separated, dried over magnesium sulfate and evaporated under reduced pressure to give an oil of 2-cyclopropyl-4,6-dichloropyrimidine. To an ice-cooled solution of 2-cyclopropyl-4,6-dichloropyrimidine in methanol (10 ml) was added dropwise slowly sodium ethoxide (1.41 g) over 45 minutes. After stirring for 10 minutes, the mixture was stirred under reflux for 2 hours. After cooling to room temperature, the insoluble materials were filtered off. The filtrate was concentrated under reduced pressure to give white solid. The solid was partitioned between 1N hydrochloric acid and dichloromethane. The organic layer was separated, dried over magnesium sulfate and evaporated. The residue was purified by column chromatography on silica gel using a mixed solvent of hexane and ethyl acetate (9:1). The fractions containing the objective compound were collected and concentrated under reduced pressure to give a syrup containing three components of 2-cyclopropyl-4,6-diethoxypyrimidine, 2-cyclopropyl-4,6-dimethoxypyrimidine and 2-cyclopropyl-4-ethoxy-6-methoxypyrimidine. The three aromatic signals in NMR (CDCl₃, δ), 5.74, 5.77, 5.79 (1:1.5:3), were assigned to that of 2-cyclopropyl-4,6-diethoxypyrimidine, 2-cyclopropyl-4-ethoxy-6-methoxypyrimidine and 2-cyclopropyl-4,6-dimethoxypyrimidine respectively.

To a cooled suspension of a syrup (1.6 g) containing three components above in tetrahydrofuran (10 ml) was added 1.5 M butyllithium in hexane (6.5 ml) dropwise over 10 minutes between −30° C. and −40° C. The whole was stirred at the same temperature for 30 minutes followed by at between −20° C. and −20° C. for 30 minutes. To the solution was added N,N-dimethylformamide (1.19 ml) at one time, and the whole was stirred at 10° C. for 10 minutes. To the solution was added 1N hydrochloric acid (10 ml) (pH ca. 5-6) and the whole was extracted with ethyl acetate (100 ml×3). The combined extract was dried over sodium sulfate, and evaporated under reduced pressure. The residue was purified by preparative TLC with eluting a mixed solvent of hexane and ethyl acetate (4:1).

(1) The first eluant was concentrated under reduced pressure to give crystals of 2-cyclopropyl-4,6-diethoxy-5-pyrimidinecarbaldehyde (0.30 g).

mp: 86-87° C.

NMR (CDCl₃, δ): 1.05-1.22 (4H, m), 1.41 (6H, t, J=7.0 Hz), 2.02-2.10 (1H, m), 4.52 (2H, q, J=7.0 Hz), 10.28 (1H, s)

MASS (API-ES): 495 (2M+Na)⁺, 259 (M+Na)⁺

(2) The second eluant was concentrated under reduced pressure to give crystals of 2-cyclopropyl-4-ethoxy-6-methoxy-5-pyrimidinecarbaldehyde (0.45 g).

mp: 93-94° C.

NMR (CDCl₃, δ): 1.05-1.22 (4H, m), 1.41 (3H, t, J=7.0 Hz), 2.02-2.10 (1H, m), 4.03 (3H, s), 4.52 (2H, q, J=7.0 Hz), 10.28 (1H, s)

MASS (API-ES): 223

(3) The last eluant was concentrated under reduced pressure to give 2-cyclopropyl-4,6-dimethoxy-5-pyrimidinecarbaldehyde

Preparation 50

To an ice-cooling solution of tert-butyl (4R, 9aS)-4-benzhydryloctahydro-2H-pyrazino[1,2-a]pyrazine-2-carboxylate (550 mg) in dichloromethane (10 ml) and 3-methoxypropanoic acid (0.139 ml) were added triethylamine (200 μl), 1-hydroxybenzotriazole (201 mg), 1-(3-dimethylaminopropyl)-3-ethylcarbodiimide hydrochloride (285 mg), and the whole was stirred overnight. The reaction mixture was washed with aqueous sodium bicarbonate, and brine successively. The organic layer was separated, dried over magnesium sulfate and evaporated under reduced pressure. The residue was purified by column chromatography on silica gel using a mixed solvent of dichloromethane and methanol (20:1). The fractions containing the objective compound were collected and concentrated under reduced pressure. The residue was treated with 4N hydrogen chloride in dioxane (1 ml), the resulting precipitate was collected by filtration with diisopropyl ether, washed with diisopropyl ether, and dried in vacuo to give a powder of (6R, 9aR)-6-benzhydryl-2-(3-methoxypropanoyl)octahydro-2H-pyrazino[1,2-a]pyrazine dihydrochloride (0.36 g).

NMR (DMSO-d₆, δ): 2.20-4.90 (20H, m), 7.15-7.41 (10H, m), 9.40 (2H, br s)

MASS (API-ES): 416 (M+Na)⁺, 394 (M+H)⁺(free)

Preparation 51

To an ice-cooling solution of tert-butyl (4R, 9aS)-4-benzhydryloctahydro-2H-pyrazino[1,2-a]pyrazine-2-carboxylate (305 mg) in dichloromethane (10 ml) and triethylamine (0.156 ml) was added dropwise methoxyacetyl chloride (89.3 mg) over 10 minutes and the whole was stirred for 3 hours. The mixture was washed with aqueous sodium bicarbonate and brine successively, dried over magnesium sulfate and evaporated under reduced pressure. The residue was purified by column chromatography on silica gel using a mixed solvent of dichloromethane and methanol (20:1). The fractions containing the objective compound were collected and concentrated under reduced pressure. The residue was treated with 4N hydrogen chloride in ethyl acetate (2.5 ml), the resulting precipitate was collected by filtration with diisopropyl ether, washed with diisopropyl ether, and dried in vacuo to give a powder of (6R, 9aR)-6-benzhydryl-2-(methoxyacetyl)octahydro-2H-pyrazino[1,2-a]pyrazine dihydrochloride (328 mg).

NMR (DMSO-d₆, δ): 2.20-4.65 (15H, m), 3.25 (3H, s), 7.21-7.45 (10H, m), 9.50-9.80 (2H, m)

MASS (API-ES): 781 (2M+Na)⁺, 402 (M+Na)⁺, 380 (M+H)⁺(free)

Preparation 52

To a suspension of 1,1′-carbonyldiimidazole (3.07 g) in tetrahydrofuran (20 ml) was added morpholine (1.5 g) at room temperature, and then the mixture was refluxed with stirring for 8 hours. After cooling, the mixture was concentrated under reduced pressure, then dichloromethane and water were added to the residue with stirring. The pH of the aqueous layer was adjusted to 8 with 1N hydrochloric acid. The organic layer was separated, dried over sodium sulfate, and concentrated under reduced pressure to give white powder of 4-(1H-imidazol-1-ylcarbonyl)morpholine (1.64 g).

NMR (CDCl₃, δ): 3.61-3.65 (4H, m), 3.68-3.79 (4H, m), 7.11 (1H, d, J=2.9 Hz), 7.21 (1H, d, J=2.9 Hz), 7.88 (1H, s).

MASS (API-ES): 182 (M+H)⁺

Preparation 53

To a solution of 4-(1H-imidazol-1-ylcarbonyl)morpholine (256 mg) in acetonitrile (3 ml) was added methyl iodide (784 mg) at room temperature, then the whole was stirred at the same temperature for 1 hour, followed by at 42° C. for 5 hours. After the reaction mixture was concentrated under reduced pressure, the residue was dissolved in dichloromethane (8 ml). To the solution were added tert-butyl (4R, 9aS)-4-benzhydryloctahydro-2H-pyrazino[1,2-a]pyrazine-2-carboxylate (500 mg) and triethylamine (124 mg) at room temperature, and the whole was stirred for overnight. The reaction mixture was poured into water, and the aqueous layer was adjusted to pH 6 with 1N hydrochloric acid. The organic layer was washed with water twice, separated, dried over sodium sulfate, and concentrated under reduced pressure. The resulting residue was purified by column chromatography on silica gel (15 g) using a mixed solvent of hexane and ethyl acetate (1:1). The fractions containing the objective compound were collected and evaporated under reduced pressure to give white foam of tert-butyl (4R, 9aS)-4-benzhydryl-8-(morpholinocarbonyl)octahydro-2H-pyrazino[1,2-a]pyrazine-2-carboxylate (585 mg).

IR (KBr): 2972, 2854, 1693, 1647, 1417, 1240, 1163, 1120 cm⁻¹

NMR (CDCl₃, δ): 1.32 (9H, br), 1.95-4.20 (21H, m), 7.10-7.40 (10H, m)

MASS (API-ES): 521 (M+H)⁺

Preparation 54

4N Hydrogen chloride in dioxane solution (2.3 ml) was added dropwise to a solution of tert-butyl (4R, 9aS)-4-benzhydryl-8-(morpholinocarbonyl)octahydro-2H-pyrazino[1,2-a]pyrazine-2-carboxylate (532 mg) in dichloromethane (3 ml) under ice-cooling. After being stirred at room temperature for 1 hour, the reaction mixture was concentrated under reduced pressure to give colorless foam of (6R, 9aR)-6-benzhydryl-2-(morpholinocarbonyl)octahydro-2H-pyrazino[1,2-a]pyrazine dihydrochloride (492 mg).

NMR (DMSO-d₆, δ): 2.30-4.70 (22H, m), 7.10-7.50 (10H, m), 9.75 (2H, br)

MASS (API-ES): 421 (M+H)⁺(free)

Preparation 55

To a solution of isoxazole-5-carboxylic acid (126 mg) in dichloromethane (10 ml) were added triethylamine (169 mg), tert-butyl (4R, 9aS)-4-benzhydryloctahydro-2H-pyrazino-[1,2-a]pyrazine-2-carboxylate (455 mg) under ice-cooling. After 2-chloro-1-methylpyridinium iodide (428 mg) was added to the solution at the same temperature, the whole was stirred at room temperature for 14 hours. The reaction mixture was poured into aqueous sodium bicarbonate, and the organic layer was separated, washed with brine, dried over sodium sulfate, and concentrated under reduced pressure. The resulting residue was purified by column chromatography on silica gel (14 g) using a mixed solvent of hexane and ethyl acetate (1:1). The fractions containing the objective compound were collected and evaporated under reduced pressure to give colorless syrup of tert-butyl (4R, 9aS)-4-benzhydryl-8-(5-isoxazolylcarbonyl)octahydro-2H-pyrazino[1,2-a]pyrazine-2-carboxylate (105 mg).

NMR (CDCl₃, δ): 1.10-1.60 (9H, m), 2.00-4.40 (13H, m), 7.10-7.40 (10H, m), 6.75 (1H, s), 8.28 (1H, m)

MASS (API-ES): 525 (M+Na)⁺

Preparation 56

The following compound was obtained according to a similar manner to that of Preparation 54.

### (6R, 9aR)-6-Benzhydryl-2-(5-isoxazolylcarbonyl)-octahydro-2H-pyrazino[1,2-a]pyrazine

MASS (API-ES): 403 (M+H)⁺(free)

Preparation 57

3-[(6R, 9aR)-6-Benzhydryloctahydro-2H-pyrazino[1,2-a]pyrazin-2-yl]-3-oxo-1-propanol dihydrochloride (200 mg) was partitioned between aqueous saturated sodium bicarbonate and dichloromethane. The organic layer was separated, dried over sodium sulfate and evaporated under reduced pressure to give a brownish oil of 3-[(6R, 9aR)-6-benzhydryloctahydro-2H-pyrazino[1,2-a]pyrazin-2-yl]-3-oxo-1-propanol (145 mg).

MASS (API-ES): 781 (2M+Na)⁺, 402 (M+Na)⁺, 380 (M+H)⁺

Preparation 58

The following compound was obtained according to a similar manner to that of Preparation 57.

### (6R, 9aR)-6-Benzhydryl-2-(methoxyacetyl)octahydro-2H-pyrazino[1,2-a]pyrazine Dihydrochloride

MASS (API-ES): 781 (2M+Na)⁺, 402 (M+Na)⁺, 380 (M+H)⁺

Preparation 59

1-[3-(Dimethylamino)propyl]-3-ethylcarbodiimide hydrochloride (2.11 g) was added over 5 minutes to a mixture of N,O-dimethylhydroxylamine hydrochloride (1.17 g), (2S)-piperazine-1,2,4-tricarboxylic acid 4-benzyl ester 1-tert-butyl ester (3.64 g), 1-hydroxybenzotriazole (1.49 g) and N,N-diisopropylethylamine (2.1 ml) in dichloromethane (40 ml). After being stirred for 18 hours at room temperature, the resulting mixture was extracted with ethyl acetate. The extract was washed with brine, dried over sodium sulfate and evaporated under reduced pressure. The residue was purified by column chromatography on silica gel using a mixed solvent of hexane and ethyl acetate (3:1) to give 2-(N-methoxy-N-methylcarbamoyl)piperazine-1,4-dicarboxylic acid 4-benzyl ester 1-tert-butyl ester (3.61 g) as a colorless powder.

NMR (CDCl₃, δ): 1.45 (9H, s), 2.90-3.20 (5H, m), 3.60-4.20 (6H, m), 4.41 (1H, m), 4.90 (1H, m), 5.06 (1H, d, J=12.4 Hz), 5.16 (1H, d, J=12.4 Hz), 7.33 (5H, m)

MASS (APCI): 308 (M−Boc+H)+

Preparation 60

Lithium aluminum hydride (38 mg) was added by small portions to an ice-cooled solution of 2-(N-methoxy-N-methylcarbamoyl)piperazine-1,4-dicarboxylic acid 4-benzyl ester 1-tert-butyl ester (407 mg) in tetrahydrofuran (5 ml) below 5° C. under nitrogen atmosphere. After the mixture was stirred at the same temperature for 2.5 hours, 2N sodium hydroxide (0.2 ml) was added to the mixture. After the mixture was stirred for 30 minutes, the insoluble materials were removed by filtration and washed with tetrahydrofuran. The filtrate and the washing were combined, and evaporated under reduced pressure to give a residue. Sodium triacetoxyborohydride (424 mg) was added portionwisely to a stirred mixture of the residue obtained in the above procedure and 2-methoxybenzylamine (151 mg) in dichloromethane (4 ml). After being stirred at room temperature for 4 hours, 3-bromo-1,1-diphenyl-2-propanone (347 mg) in N,N-dimethylformamide (5 ml) and N,N-diisopropylethylamine (0.35 ml) were added successively to the reaction mixture at 5° C. The whole mixture was stirred at room temperature for 36 hours and then poured into ice-water, and extracted with ethyl acetate. The extract was washed with brine, dried over magnesium sulfate and evaporated under reduced pressure. The residue was purified by column chromatography on silica gel using a mixed solvent of hexane and ethyl acetate (4:1) to give (2R)-2-[[N-(2-methoxybenzyl)-N-(2-oxo-3,3-diphenylpropyl)amino]methyl]-piperazine-1,4-dicarboxylic acid 4-benzyl ester 1-tert-butyl ester (170 mg) as a colorless powder.

NMR (CDCl₃, δ): 1.41-1.57 (9H, m), 2.70-3.00 (5H, m), 3.25-4.35 (11H, m), 4.95-5.15 (3H, m), 6.70-7.29 (19H, m)

Preparation 61

4N Hydrogen chloride in ethyl acetate solution (3 ml) was added to a solution of (2R)-2-[[N-(2-methoxybenzyl)-N-(2-oxo-3,3-diphenylpropyl)amino]methyl]piperazine-1,4-dicarboxylic acid 4-benzyl ester 1-tert-butyl ester (160 mg) in ethyl acetate (3 ml) at room temperature. After being stirred for 2 hours, the reaction mixture was concentrated under reduced pressure. The resulting residue was dissolved into dichloromethane (4 ml). Sodium triacetoxyborohydride (150 mg) was added to the stirred mixture and the whole was stirred at room temperature for 18 hours. The mixture was partitioned between ethyl acetate and 2N sodium hydroxide. The organic layer was separated, washed with brine, dried over magnesium sulfate and evaporated under reduced pressure. The resulting residue was purified by column chromatography on silica gel using a mixed solvent of hexane and ethyl acetate (3:4) as an eluent to give (6R, 9aR)-6-benzhydryl-8-(2-methoxybenzyl)octahydropyrazino[1,2-a]pyrazine-2-carboxylic acid benzyl ester (108 mg) as a colorless powder.

NMR (CDCl₃, δ): 1.83-2.09 (3H, m), 2.43 (2H, m), 2.60-3.05 (4H, m), 3.20-3.56 (3H, m), 3.68 (3H, s), 3.78 (2H, m), 4.18 (1H, d, J=6.9 Hz), 5.08 (2H, s), 6.70-7.32 (19H, m)

MASS (APCI): 562 (M+H)⁺

Preparation 62

A solution of (6R, 9aR)-6-benzhydryl-8-(2-methoxybenzyl)octahydropyrazino[1,2-a]pyrazine-2-carboxylic acid benzyl ester (100 mg) and triethylamine (0.049 ml) in tetrahydrofuran (3 ml) was hydrogenated over 10% palladium-carbon (50% wet, 20 mg) at room temperature under atmospheric pressure for 2 hours. After removal of the catalyst by filtration, the filtrate was evaporated under reduced pressure to give an oil, which was purified by column chromatography on silica gel using a mixed solvent of dichloromethane and methanol (4:1). The fractions containing the objective compound were collected and evaporated under reduced pressure and the resulting residue was treated with 4N hydrogen chloride in ethyl acetate to give (6R, 9aS)-4-benzhydryl-2-(2-methoxybenzyl)-octahydropyrazino[1,2-a]pyrazine trihydrochloride (58 mg) as a colorless powder.

NMR (DMSO-d₆, δ): 2.26-4.45 (19H, m), 6.91-7.46 (14H, m)

MASS (APCI): 428 (M+H)⁺(free)

Preparation 63

The following compound was obtained according to a similar manner to that of Preparation 62.

### (4R, 9aS)-4-Benzhydryl-2-(2-methoxybenzyl)octahydro-2H-pyrazino[1,2-a]pyrazine

NMR (CDCl₃, δ): 3.67 (3H, s), 1.50-4.30 (16H, m), 6.70-6.90 (2H, m), 7.10-7.35 (12H, m)

MASS (APCI): 428 (M+H)⁺

Preparation 64

Acetyl chloride (3 drops) was added to a mixture of (6R, 9aS)-4-benzhydryl-2-(2-methoxybenzyl)octahydropyrazino-[1,2-a]pyrazine trihydrochloride (20 mg) and N,N-diisopropylethylamine (6 drops) in dichloromethane (1 ml) under ice-cooling. After being stirred at the same temperature for 2 hours, the mixture was poured into ice-water and extracted with ethyl acetate. The extract was washed with brine, dried over sodium sulfate and evaporated under reduced pressure to give a crude oil. The oil was purified by column chromatography on silica gel using a mixed solvent of dichloromethane and methanol (50:1) as an eluent. The fractions containing the objective compound were collected and evaporated under reduced pressure and the resulting residue was treated with 4N hydrogen chloride in ethyl acetate to give 1-[(6R, 9aR)-6-benzhydryl-8-(2-methoxybenzyl)octahydropyrazino[1,2-a]pyrazin-2-yl]ethanone dihydrochloride (9.8 mg) as a colorless powder.

NMR (DMSO-d₆, δ): 1.90-4.60 (21H, m), 6.95-7.39 (14H, m)

MASS (APCI): 470 (M+H)⁺(free)

Preparation 65

The following compound was obtained according to a similar manner to that of Preparation 64.

### (4R, 9aR)-8-Acetyl-4-benzhydryl-2-(2-methoxybenzyl)-octahydro-2H-pyrazino[1,2-a]pyrazine

NMR (CDCl₃, δ): 3.60-3.70 (3H, m), 1.70-4.00 (16H, m), 4.05-4.30 (2H, m), 6.70-6.95 (2H, m), 7.09-7.35 (12H, m)

MASS (APCI): 470 (M+H)⁺

Preparation 66

To a solution of (4R, 9aR)-8-acetyl-4-benzhydryl-2-(2-methoxybenzyl)octahydro-2H-pyrazino[1,2-a]pyrazine (5.9 g) in dichloroethane (60 ml) was added 1-chloroethyl chloroformate (2.3 ml) at room temperature, and the reaction mixture was heated at 70° C. for 30 minutes with stirring. After removal of solvent by evaporation, to the resulting residue was added methanol (45 ml), and the solution was refluxed for 40 minutes. After being concentrated, the residue was triturated with diisopropyl ether. The resulting precipitate was collected by filtration and dried under reduced pressure for 5 hours at 40° C. to give (4R, 9aR)-8-acetyl-4-benzhydryloctahydro-2H-pyrazino[1,2-a]pyrazine dihydrochloride (3.1 g) as a colorless foam.

NMR (DMSO-d₆, δ): 1.90-2.00 (3H, m), 2.20-4.70 (13H, m), 7.10-7.50 (10H, m), 9.65 (2H, br)

MASS (APCI): 350 (M+H)⁺(free)

Preparation 67

Under nitrogen atmosphere, to a solution of (2S)-2-ethoxycarbonylpiperazine-1,4-dicarboxylic acid 4-benzyl ester 1-tert-butyl ester (9.35 g) was added portionwise lithium borohydride (1.82 g), and the reaction mixture was stirred for 90 minutes. After methanol (2.32 ml) was added dropwise to the solution under ice-cooling, the mixture was stirred at room temperature for 17 hours. 1N Hydrochloric acid (80 ml) was added dropwise under ice-cooling, and ethyl acetate (100 ml) and sodium chloride (6 g) was added to it. The organic layer was washed with brine, dried over magnesium sulfate, and evaporated under reduced pressure to give colorless oil. The oil was purified by column chromatography on silica gel (90 g) using a mixed solvent of hexane and ethyl acetate (3:2). The fractions containing the objective compound were collected and evaporated under reduced pressure to give (2S)-2-(hydroxymethyl)piperazine-1,4-dicarboxylic acid 4-benzyl ester 1-tert-butyl ester (8.40 g) as a colorless oil.

NMR (CDCl₃, δ): 1.46 (9H, s), 2.40-4.30 (10H, m), 5.10-5.30 (2H, m), 7.30-7.50 (5H, m)

MASS (API-ES): 373 (M+Na)⁺

Preparation 68

The following compound was obtained accordidng to a similar manner to that of Preparation 67.

### 4-Benzyl 1-tert-butyl (2S)-2-(hydroxymethyl-1,4-piperazinedicarboxylate

NMR (CDCl₃, δ): 1.46 (9H, s), 2.52 (1H, br), 2.91-3.00 (3H, m), 3.58 (2H, m), 3.84-4.17 (4H, m), 5.15 (2H, s), 7.35-7.45 (5H, m)

Preparation 69

Under nitrogen atmosphere, to a solution of oxalyl chloride (1.64 ml) in dichloromethane (34 ml) under −65° C., was added dropwise a solution of dimethyl sulfoxide (2.0 ml) in dichloromethane (15 ml) and stirred for 10 minutes at the same temperature. A solution of (2S)-2-(hydroxymethyl)-piperazine-1,4-dicarboxylic acid 4-benzyl ester 1-tert-butyl ester (3.29 g) in dichloromethane (24 ml) was dropped into the above solution over 5 minutes under −65° C. The reaction mixture was stirred at the same temperature for 15 minutes, then stirred at −45° C. for 90 minutes. Triethylamine (7.85 ml) was added to the solution under −40° C., and the mixture was stirred at 0° C. for 20 minutes. The mixture was poured into saturated aqueous ammonium chloride (100 ml). The organic layer was washed with brine, dried over magnesium sulfate, and evaporated to give (2R)-2-formylpiperazine-1,4-dicarboxylic acid 4-benzyl ester 1-tert-butyl ester (3.33 g) as a colorless syrup.

NMR (CDCl₃, δ): 1.40-1.70 (9H, m), 2.85-3.30 (3H, m), 3.70-4.80 (4H, m), 5.05-5.30 (2H, m), 7.30-7.40 (5H, m), 9.58 (1H, s)

MASS (API-ES): 371 (M+Na)⁺

Preparation 70

The following compound was obtained according to a similar manner to that of Preparation 69.

### 4-Benzyl 1-tert-butyl (2S)-2-formyl-1,4-piperazinedicarboxylate

MASS (ESI, negative): 347 (M−H)

Preparation 71

Under nitrogen atmosphere, to a solution of (2R)-2-formylpiperazine-1,4-dicarboxylic acid 4-benzyl ester 1-tert-butyl ester (2.64 g) and 3-(2-methoxybenzylamino)-1,1-diphenylpropan-2-one (3.66 g) in dichloromethane (30 ml) was added acetic acid (0.607 ml) and sodium tritacetoxyborohydride (4.82 g) under ice-cooling, and then it was stirred at room temperature for 3 hours. The reaction mixture was poured into aqueous sodium bicarbonate (100 ml) and extracted with dichloromethane. The organic layer was washed with brine, dried over sodium sulfate, and evaporated under reduced pressure. The resulting residue was purified by column chromatography on silica gel (82 g) using a mixed solvent of hexane and ethyl acetate (3:1). The fractions containing the objective compound were collected and evaporated under reduced pressure to give (2S)-2-[[N-(2-methoxybenzyl)-N-(2-oxo-3,3-diphenylpropyl)amino]methyl]piperazine-1,4-dicarboxylic acid 4-benzyl ester 1-tert-butyl ester (3.24 g) as a syrup.

NMR (CDCl₃, δ): 1.40-1.65 (9H, m), 2.65-5.40 (19H, m), 6.70-7.40 (19H, m)

MASS (APCI): 678 (M+H)⁺

Preparation 72

The following compound was obtained according to a similar manner to that of Preparation 71.

### 4-Benzyl 1-tert-butyl (2R)-2-[[N-(2-methoxybenzyl)-N-(2-oxo-3,3-diphenylpropyl)amino]methyl]-1,4-piperazinedicarboxylate

NMR (CDCl₃, δ): 1.41 (9H, s), 2.70-5.52 (19H, m), 6.73-7.29 (19H, m)

MASS (ESI): 678 (M+H)

Preparation 73

To a solution of (2S)-2-[[N-(2-methoxybenzyl)-N-(2-oxo-3,3-diphenylpropyl)amino]methyl]piperazine-1,4-dicarboxylic acid 4-N-benzyl ester 1-N-tert-butyl ester (3.15 g) in ethyl acetate (15 ml) was added a solution of 4N hydrogen chloride in ethyl acetate (29.6 ml) under ice-cooling. After stirring at the same temperature for 3 hours, the reaction mixture was evaporated under reduced pressure. To the solution of the residue in dichloromethane (30 ml) was added portionwise sodium triacetoxyborohydride (2.95 g) under ice-cooling, and then it was stirred at the same temperature for 20 hours. The mixture was poured into aqueous sodium bicarbonate and extracted with dichloromethane. The organic layer was washed with brine, dried over sodium sulfate, evaporated under reduced pressure. The resulting residue was purified by column chromatography on silica gel (5.2 g) using a mixed solvent of hexane and ethyl acetate (2:1). The fractions containing the objective compound were collected and evaporated under reduced pressure to give (4S, 9aS)-8-(benzyloxycarbonyl)-4-benzhydryl-2-(2-methoxybenzyl)octahydro-2H-pyrazino[1,2-a]pyrazine (2.0 g) as a syrup.

NMR (CDCl₃, δ): 3.68 (3H, s), 1.75-4.25 (15H, m), 5.08 (2H, s), 6.70-6.90 (2H, m), 7.10-7.40 (17H, m)

MASS (APCI): 562 (M+H)⁺

Preparation 74

The following compound was obtained according to a similar manner to that of Preparation 73.

### Benzyl (6R, 9aR)-6-benzhydryl-8-(2-methoxybenzyl)-octahydro-2H-pyrazino[1,2-a]pyrazine-2-carboxylate

NMR (CDCl₃, δ): 1.88 (2H, m), 2.03 (1H, m), 2.49 (2H, m), 2.68 (2H, m), 2.91 (2H, m), 3.28-3.42 (3H, m), 3.67 (3H, s), 3.67-3.78 (2H, m), 4.17 (1H, d, J=5.7 Hz), 5.07 (2H, s), 6.76-6.85 (2H, m), 7.11-7.37 (17H, m)

MASS (APCI): 562 (M+H)

Preparation 75

To a solution of (4R, 9aR)-8-acetyl-4-benzhydryl-2-(2-methoxybenzyl)octahydro-2H-pyrazino[1,2-a]pyrazine (5.9 g) in dichloroethane (60 ml) was added 1-chloroethyl chloroformate (2.3 ml) at room temperature, and the reaction mixture was heated at 70° C. for 30 minutes with stirring. After removal of solvent by evaporation, to the resulting residue was added methanol (45 ml), and the solution was refluxed for 40 minutes. After being concentrated, the residue was triturated with diisopropyl ether. The resulting precipitate was collected by filtration and dried under reduced pressure for 5 hours at 40° C. to give (4R, 9aR)-8-acetyl-4-benzhydryloctahydro-2H-pyrazino[1,2-a]pyrazine dihydrochloride (3.1 g) as colorless foam.

NMR (DMSO-d₆, δ): 1.90-2.00 (3H, m), 2.20-4.70 (13H, m), 7.10-7.50 (10H, m), 9.65 (2H, br)

MASS (APCI): 350 (M+H)⁺(free)

Preparation 76

The following compound was obtained according to a similar manner to that of Preparation 75.

### (6R, 9aR)-6-Benzhydryloctahydro-2H-pyrazino[1,2-a]pyrazine-2-carboxylate Dihydrochloride

[□]_{D}^{23.8} −60.4411 (C=0.34, MeOH 68 mg in 2 ml)

mp: 235-236° C.

IR (KBr): 3423, 2588, 2467, 2441, 1703, 1423, 1265, 1230, 1163, 1142, 1049 cm⁻¹

NMR (DMSO-d₆-D₂O, δ): 2.40-3.80 (11H, m), 4.22-4.58 (2H, m), 5.08 (2H, s), 7.14-7.52 (15H, m)

MASS (ES): 442.3 (M+H)⁺(free)

Preparation 77

To a solution of benzyl (6R, 9aR)-6-benzhydryloctahydro-2H-pyrazino[1,2-a]pyrazine-2-carboxylate dihydrochloride (10.01 g) and triethylamine (4.13 g) in dichloromethane (50 ml) was added di-tert-butyldicarbonate (4.46 g) at room temperature and stirred at the same temperature for 1.5 hours. The mixture was poured into water (50 ml) and the pH of the aqueous layer was adjusted to 5 with 1N hydrochloric acid. The organic layer was separated, washed with brine, dried over sodium sulfate, and concentrated under reduced pressure. The resulting residue was purified by column chromatography on silica gel (120 g) using a mixed solvent of hexane and ethyl acetate (1:3). The fractions containing the objective compound were collected and evaporated under reduced pressure to give 8-benzyl 2-tert-butyl (4R, 9aS)-4-benzhydrylhexahydro-2H-pyrazino[1,2-a]pyrazine-2,8(1H)-dicarboxylate (10.6 g).

NMR (CDCl₃, δ): 1.32 (9H, br), 1.80-4.20 (13H, m), 5.09 (2H, s), 7.10-7.45 (15H, m)

MASS (API-ES): 542 (M+H)⁺

Preparation 78

A solution of 8-benzyl 2-tert-butyl (4R, 9aS)-4-benzhydrylhexahydro-2H-pyrazino[1,2-a]pyrazine-2,8(1H)-dicarboxylate (11.0 g) in methanol (110 ml) was hydrogenated over 10% palladium on activated carbon (50% wet, 2.8 g) at room temperature under atmospheric pressure for 4 hours. After removal of the catalyst by filtration, the filtrate was evaporated under reduced pressure to give tert-butyl (4R, 9aS)-4-benzhydryloctahydro-2H-pyrazino[1,2-a]pyrazine-2-carboxylate (8.0 g) as an oil.

MASS (API-ES): 408 (M+H)⁺

Preparation 79

The following compound was obtained according to a similar manner to that of Preparation 64.

### tert-Butyl (4R, 9aS)-4-benzhydryl-8-[(benzyloxy)-acetyl]octahydro-2H-pyrazino[1,2-a]pyrazine-2-carboxylate

NMR (CDCl₃, δ): 1.33 (9H, br s), 1.90-4.30 (15H, m), 4.54-4.57 (2H, m), 7.17-7.34 (15H, m)

MASS (ESI): 556 (M+H)⁺

Preparation 80

To a solution of tert-butyl (4R, 9aS)-4-benzhydryl-8-[(benzyloxy)acetyl]octahydro-2H-pyrazino[1,2-a]pyrazine-2-carboxylate (499.6 mg) in dichloromethane (2.5 ml) was added trifluoroacetic acid (2.5 ml) at 0° C. Then the mixture was stirred at room temperature for 1.5 hours and evaporated to dryness. The residue was added aqueous saturated sodium bicarbonate (20 ml) and extracted with ethyl acetate (×3). The combined organic extracts were dried over sodium sulfate and evaporated under reduced pressure to give (6R, 9aR)-6-benzhydryl-2-[(benzyloxy)acetyl]octahydro-2H-pyrazino[1,2-a]pyrazine (467.6 mg) as an oil.

NMR (CDCl₃, δ): 1.93-4.22 (15H, m), 4.54 (2H, s), 7.11-7.34 (15H, m)

MASS (APCI): 456 (M+H)⁺

Preparation 81

A mixture of (6R, 9aR)-6-benzhydryl-2-[(benzyloxy)-acetyl]octahydro-2H-pyrazino[1,2-a]pyrazine (450 mg), 20% palladium hydroxide on carbon (120 mg) and concentrated hydrochloric acid (0.146 ml) in methanol (10 ml) was hydrogenated with 3 atmospheric hydrogen at room temperature for 2 hours. And then to the mixture was added additional 20% palladium hydroxide on carbon (120 mg), and the mixture was hydrogenated under the same condition for 18 hours. The mixture was filtered, and the filtrate was evaporated under reduced pressure to give 2-[(6R, 9aR)-6-benzhydryloctahydro-2H-pyrazino[1,2-a]pyrazin-2-yl]-2-oxoethanol dihydrochloride (372.8 mg) as a solid.

NMR (DMSO-d₆, δ): 2.30-5.20 (15H, m), 7.18-7.45 (10H, m)

MASS (APCI): 366 (M+H)⁺(free)

Preparation 82

2-[(6R, 9aR)-6-Benzhydryloctahydro-2H-pyrazino[1,2-a]pyrazin-2-yl]-2-oxoethanol dihydrochloride (200 mg) was partitioned between aqueous saturated sodium bicarbonate and dicloromethane. The organic layer was separated, washed with brine, dried over magnesium sulfate and evaporated under reduced pressure to give 2-[(6R, 9aR)-6-benzhydryloctahydro-2H-pyrazino[1,2-a]pyrazin-2-yl]-2-oxoethanol (170 mg) as an oil.

MASS (APCI): 366 (M+H)⁺

Preparation 83

To an ice-cooling mixture of (4R, 9aS)-4-benzhydrylocathydro-2H-pyrazino[1,2-a]pyrazine-2-carboxylate (407 mg), triethylamine (0.21 ml) and nicotinic acid (123 mg) in dichloromethane (20 ml) was added 2-chloro-1-methylpyridium iodide (255 mg), and the whole was stirred at room temperature for 14 hours. The mixture was washed with aqueous sodium bicarbonate and water successively, dried over sodium sulfate and evaporated under reduced pressure. The residue was purified by column chromatography on silica gel using a mixed solvent of dichloromethane and methanol (40:1). The fractions containing the objective compound were collected and concentrated under reduced pressure to give a syrup of tert-butyl (4R, 9aS)-4-benzhydryl-8-(3-pyridylcarbonyl)octahydro-2H-pyrazino[1,2-a]pyrazine-2-carboxylate (300 mg).

NMR (CDCl₃, δ): 1.31 (9H, s), 1.50-4.30 (13H, m), 7.10-7.40 (11H, m), 7.70-7.75 (1H, m), 8.61-8.66 (2H, m)

MASS (APCI): 535 (M+Na), 513 (M+H)⁺

Preparation 84

4N Hydrogen chloride in 1,4-dioxane (44 ml) was added to a solution of 4-tert-butoxycarbonyl-2-benzhydryl-1-methylpiperazine (6.5 g) in ethanol (33 ml) under ice-cooling over 30 minutes. The mixture was stirred at room temperature for 4 hours and evaporated under reduced pressure. The residue was triturated with diisopropyl ether and the resulting solid was collected by filtration to give 2-benzhydrylpiperazine dihydrochloride (6.02 g) as a powder.

NMR (DMSO-d₆, δ): 2.50-3.95 (6H, m), 3.56 (3H, s), 4.30-5.50 (2H, m), 7.21-7.57 (11H, m)

MASS (APCI): 267 (M+H)⁺(free)

Preparation 85

The following compound was obtained according to a similar manner to that of Preparation 84.

### (6R, 9aR)-6-Benzhydryl-2-(3-pyridylcarbonyl)octahydro-2H-pyrazino[1,2-a]pyrazine Trihydrochloride

MASS (ES): 413 (M+H)⁺(free)

Preparation 86

To a suspension of 1-acetylamino-1-cyclopropanecarboxylic acid (31.7 mg) in dichloromethane (3 ml) were added triethylamine (46.4 μl) and 2-chloro-1-methylpyridinium iodide (85 mg) at room temperature. After being stirred for 30 minutes, (4R, 9aS)-4-benzhydryl-2-[2-methoxy-5-[5-(trifluoromethyl)-1H-tetrazol-1-yl]benzyl]-octahydro-2H-pyrazino[1,2-a]pyrazine (125 mg) was added to the solution at the same temperature, and the whole was stirred at room temperature for 14 hours. After removal of solvent by evaporation; to the resulting residue were added N,N-dimethylformamide (3.5 ml) and triethylamine (15 μl), and the whole mixture was heated at 90° C. for 3 hours with stirring. The solution was partitioned between ethyl acetate and water, while aqueous layer was adjusted at pH 9 with aqueous saturated sodium bicarbonate. The organic layer was separated, washed with brine, dried over sodium sulfate, and concentrated under reduced pressure. The resulting residue was purified by column chromatography on silica gel (6 g) using a mixed solvent of toluene and ethyl acetate (35:1). The fractions containing the objective compound were collected and evaporated under reduced pressure to give a syrup. To a solution of the syrup in dichloromethane (3 ml) was added a solution of 4N hydrogen chloride in ethyl acetate (50 μl), and triturated with diisopropyl ether. The precipitate was collected by filtration and dried under reduced pressure for 5 hours at 40° C. to give (4R, 9aR)-8-(1-acetylamino-1-cyclopropanecarbonyl)-4-benzhydryl-2-[2-methoxy-5-[5-(trifluoromethyl)-1H-tetrazol-1-yl]benzyl]octahydro-2H-pyrazino[1,2-a]pyrazine dihydrochloride (47 mg) as colorless powder.

IR (KBr): 3435, 1658, 1649, 1506, 1450, 1265, 1201, 1163 cm⁻¹

NMR (DMSO-d₆, δ): 0.70-0.90 (2H, m), 1.00-1.20 (2H, m), 1.73 (3H, s), 3.82 (3H, s), 2.10-4.50 (15H, m), 7.10-7.50 (11H, m), 7.70-7.90 (2H, m), 8.51 (1H, S)

MASS (APCI): 689 (M+H)⁺(free)

Preparation 87

The title compound was obtained according to a similar manner to that of Preparation 86 followed by Preparation 84.

### (6R, 9aR)-6-Benzhydryl-2-(2-pyridylcarbonyl)octahydro-2H-pyrazino[1,2-a]pyrazine Trihydrochloride

NMR (DMSO-d₆, δ): 2.10-5.20 (13H, m), 7.20-7.70 (12H, m), 7.90-8.00 (1H, m), 8.50-8.55 (1H, m), 9.63 (3H, br s)

MASS (APCI): 413 (M+H)⁺(free)

Preparation 88

To an ice-cooling mixture of tert-butyl (4R, 9aS)-4-benzhydryloctahydro-2H-pyrazino[1,2-a]pyrazine-2-carboxylate (0.5 g) in a mixture of tetrahydrofuran (10 ml) and saturated aqueous sodium bicarbonate were added 3-chloro-3-oxopropyl acetate (0.35 ml) in tetrahydrofuran (2 ml) over 10 minutes. After stirring for 30 minutes at the same temperature, the reaction mixture was extracted with ethyl acetate. The extract was dried over magnesium sulfate and evaporated under reduced pressure. The residue was dissolved into methanol (10 ml) and thereto was added 1N sodium hydroxide (1.2 ml) and the whole was stirred for 1 hour. After removal of the solvent, the residue was partitioned between water and dichloromethane. The organic layer was separated, dried over magnesium sulfate and evaporated under reduced pressure. The residue was purified by column chromatography on silica gel using a mixed solvent of dichloromethane and methanol (40:1). The fractions containing the objective compound were collected and concentrated under reduced pressure to give an intermediate of tert-butyl (4R, 9aS)-8-(3-acetoxypropionyl)-4-benzhydryloctahydro-2H-pyrazino[1,2-a]pyrazine-2-carboxylate.

NMR (CDCl₃, δ): 1.32 (9H, br s), 1.80-4.30 (20H, m), 7.19-7.30 (10H, m)

MASS (API-ES): 524 (M+Na)⁺, 502 (M+H)⁺

The intermediate above was treated with 4N hydrogen chloride in dioxane (5 ml), and the resulting precipitate was collected by filtration with diisopropyl ether, washed with diisopropyl ether, and dried in vacuo to give powders of 3-[(6R, 9aR)-6-benzhydryloctahydro-2H-pyrazino[1,2-a]pyrazin-2-yl]-3-oxo-1-propanol dihydrochloride (0.47 g).

MASS (API-ES): 402 (M+Na)⁺, 380 (M+H)⁺

Preparation 89

1-[3-(Dimethylamino)propyl]-3-ethylcarbodiimide hydrochloride (1.15 g) was added over 5 minutes to a mixture of N-(2-methoxybenzyl)glycine methyl ester hydrochloride (1.72 g), N-(tert-butoxycarbonyl)-3,3-diphenyl-L-alanine (1.71 g), 1-hydroxybenzotriazole (0.81 g) and N,N-diisopropylethylamine (1.22 ml) in dichloromethane (40 ml). After being stirred for 3 hours at room temperature, the resulting mixture was extracted with ethyl acetate. The extract was washed with brine, dried over sodium sulfate and evaporated under reduced pressure. The residue was purified by column chromatography on silica gel using a mixed solvent of hexane and ethyl acetate (4:1) to give N-[(2S)-2-(tert-butoxycarbonylamino)-3,3-diphenylpropionyl]-N-(2-methoxybenzyl)glycine methyl ester (2.34 g) as a colorless powder.

NMR (CDCl₃, δ): 1.29 (9H, s), 3.62-3.77 (6H, m), 3.89 (1H, m), 4.13 (1H, m), 4.51 (2H, m), 4.86-5.07 (1H, m), 5.30-5.68 (1H, m), 6.44-7.38 (15H, m)

MASS (APCI): 555 (M+Na)⁺

Preparation 90

The following compound was obtained according to a similar manner to that of Preparation 89.

### N-Benzyl N-[(2R)-2-tert-butoxycarbonylamino-3,3-diphenylpropionyl]glycine ethyl ester

NMR (CDCl₃, δ): 1.15-1.47 (12H, m), 3.61-4.25 (4H, m), 4.48-4.76 (2H, m), 4.99-5.17 (1H, m), 5.36-5.61 (1H, m), 6.61-7.43 (15H, m)

MASS (APCI): 417 (M+H)⁺

Preparation 91

4N Hydrogen chloride in ethyl acetate solution (10 ml) was added to a solution of N-[(2S)-2-(tert-butoxycarbonylamino)-3,3-diphenylpropionyl]-N-(2-methoxybenzyl)glycine methyl ester (1.34 g) in ethyl acetate (5 ml) at room temperature. After being stirred for 2 hours, the reaction mixture was concentrated under reduced pressure. The resulting residue was dissolved into isopropyl alcohol (8 ml) and the solution was stirred for 3 hours under reflux. After being cooled with ice, the residue was triturated with diisopropyl ether (50 ml) and the resulting solid was collected by filtration to give (3S)-3-benzhydryl-1-(2-methoxybenzyl)piperazine-2,5-dione (785 mg) as a colorless powder.

NMR (DMSO-d₆, δ): 3.01 (1H, d, J=17.4 Hz), 3.50 (1H, d, J=17.4 Hz), 3.75 (3H, s), 4.24 (1H, d, J=15.0 Hz), 4.38 (1H, d, J=15.0 Hz), 4.53 (1H, d, J=5.4 Hz), 4.73 (1H, d, J=5.4 Hz), 6.85-7.33 (14H, m), 8.39 (1H, m)

MASS (APCI): 423 (M+Na)⁺

Preparation 92

The following compound was obtained according to a similar manner to that of Preparation 91.

### (3R)-3-Benzhydryl-1-benzylpiperazine-2,5-dione

NMR (DMSO-d₆, δ): 2.98 (1H, d, J=17.2 Hz), 3.47 (1H, d, J=17.2 Hz), 4.16 (1H, d, J=14.5 Hz), 4.54 (1H, d, J=5.4 Hz), 4.57 (1H, d, J=14.5 Hz), 4.76 (1H, dd, J=5.4, 5.4 Hz), 7.07-7.41 (15H, m), 8.40 (1H, m)

MASS (APCI): 371 (M+H)⁺

Preparation 93

Lithium aluminum hydride (198 mg) was added by small portions to an ice-cooled solution of 1,4-dibenzyl 3-benzhydryl-2,5-piperazinedione (800 mg) in tetrahydrofuran (8 ml) under nitrogen atmosphere, and the mixture was stirred under reflux for 5 hours. After being cooled with ice, 2N sodium hydroxide (1 ml) was added to the mixture under nitrogen atmosphere. The resulting precipitates were filtered off and washed with tetrahydrofuran, and the filtrate and the washings were combined and evaporated under reduced pressure to give a crude oil. The oil was purified by column chromatography on silica gel using a mixed solvent of hexane and ethyl acetate (9:1). The fractions containing the objective compound were collected, evaporated under reduced pressure and treated with 4N hydrogen chloride in ethyl acetate solution to give 1,4-dibenzyl 2-benzhydrylpiperazine dihydrochloride (846 mg) as a colorless powder.

NMR (DMSO-d₆, δ): 2.30-6.50 (12H, m), 7.03-7.98 (20H, m)

MASS (APCI): 433 (M+H)⁺(free)

Preparation 94

The following compound was obtained according to a similar manner to that of Preparation 93.

### (R)-2-Benzhydryl-4-benzylpiperazine

mp: 133-135° C.

IR (KBr): 1491, 1448, 1138 cm⁻¹

NMR (CDCl₃, δ): 1.86-2.15 (2H, m) 2.57-2.95 (4H, m), 3.28 (1H, d, J=13.0 Hz), 3.46-3.68 (1H, m), 3.56 (1H, d, J=13.0 Hz), 3.83 (1H, d, J=10.5 Hz), 7.05-7.45 (15H, m)

MASS (ES): 365 (M+Na)⁺, 343 (M+H)⁺

Preparation 95

To a solution of (R)-2-benzhydryl-4-benzylpiperazine (4.57 g) in a mixture of acetone (25 ml) and tetrahydrofuran (40 ml) were added triethylamine (2.42 ml) and water (30 ml). Di-tert-butyl dicarbonate (3.49 g) was added to the reaction mixture with water bath cooling and the whole was stirred overnight. Sodium chloride and isopropyl ether were added to the mixture and the organic layer was separated, washed with brine, dried over magnesium sulfate, and evaporated in vacuo. The residue was triturated with hexane to give (R)-2-benzhydryl-4-benzylpiperazine-1-carboxylic acid tert-butyl ester (4.545 g) as a powder. The filtrate was concentrated under reduced pressure and the residue was purified by column chromatography on silica gel (hexane:ethyl acetate 1:0 to 10:1 as eluent) to give the second crop (0.837 g).

mp: 108.5-109° C.

IR (KBr): 1687, 1421, 1363, 1172, 1147 cm⁻¹

NMR (CDCl₃, δ): 1.29 and 1.38 (9H, s), 1.90-2.15 (2H, m), 2.55-4.05 (6H, m), 4.70-5.06 (2H, m), 7.02-7.52 (15H, m)

MASS (ES): 466 (M+Na)⁺, 443 (M+H)⁺

Preparation 96

To a solution of (R)-2-benzhydryl-4-benzylpiperazine-1-carboxylic acid tert-butyl ester (5.30 g) in a mixture of tetrahydrofuran (53 ml) and methanol (53 ml) was added 10% palladium hydroxide on carbon (0.53 g) and the mixture was hydrogenated with 3 atmospheric hydrogen at 40° C. for 20 hours. After cooling, the mixture was filtered and the filtrate was evaporated in vacuo to give (R)-2-benzhydrylpiperazine-1-carboxylic acid tert-butyl ester (4.49 g).

mp: 100-105° C.

NMR (CDCl₃, δ): 1.28 and 1.43 (9H, s), 2.55-4.05 (6H, m), 5.70-5.10 (2H, m), 7.05-7.50 (10H, m)

MASS (APCI): 343 (M+H)⁺

MASS (ES): 375 (M+Na)⁺, 353 (M+H)⁺, 297 (M−tBu)⁺

Preparation 97

A solution of dimethyl sulfoxide (0.219 ml) in dichloromethane (1.1 ml) was added dropwise to a solution of oxalyl chloride (0.133 ml) in dichloromethane (2.7 ml) under cooling below −60° C. with dry ice-acetone. After 5 minutes, the mixture was allowed to −10° C., and a solution of (2S)-1-benzyl 2-(hydroxymethyl)piperidine (156.5 mg) in dichloromethane (1.6 ml) was added to the mixture. The whole mixture was then cooled below −60° C. and was stirred for 20 minutes at the same temperature. After addition of triethylamine (0.64 ml) followed by stirring at room temperature, the reaction mixture was poured into water and extracted with 1,2-dichloroethane. The extract was dried over magnesium sulfate and evaporated under reduced pressure to give a syrup. Benzylamine (0.33 ml) was added to the solution of the syrup obtained above procedure in 1,2-dichloroethane (2.5 ml) with ice-cooling. After the whole was stirred for 30 minutes at the same temperature, sodium triacetoxyborohydride (0.323 g) was added to this mixture. The reaction mixture was allowed to room temperature and was stirred for 3 hours. The mixture was poured into aqueous saturated sodium bicarbonate solution and extracted with dichloromethane. The extract was dried over magnesium sulfate and evaporated under reduced pressure. The resulting residue was purified by silica gel chromatography using a mixture of dichloromethane and methanol (20:1) as an eluent to give N-benzyl [(2S)-1-benzylpiperidin-2-ylmethyl]amine (168.5 mg).

NMR (CDCl₃, δ): 1.26-1.49 (3H, m), 1.56-1.67 (3H, m), 2.03 (1H, s), 2.04-2.14 (1H, m), 2.42-2.50 (1H, m), 2.66-2.86 (3H, m), 3.25 (1H, d, J=13.6 Hz), 3.73 (2H, s), 3.92 (1H, d, J=13.6 Hz), 7.19-7.38 (20H, m)

MASS (APCI): 295 (M+H)⁺

Preparation 98

The following compound was obtained according to a similar manner to that of Preparation 97.

### Benzyl (2S, 4R)-2-[(benzylamino)methyl]-4-[[tert-butyl(dimethyl)silyl]oxy]-1-pyrrolidinecarboxylate

IR (Neat): 1702, 1422, 1504 cm⁻¹

NMR (CDCl₃, δ): 0.89 (9H, s), 0.13 (6H, s), 1.90-2.00 (2H, m), 2.70-2.85 (2H, m), 3.40-3.50 (2H, m), 3.70-3.85 (2H, m), 4.11 (1H, br s), 4.35-4.45 (1H, m), 5.05-5.20 (2H, m), 7.16-7.35 (10H, m)

MASS (APCI): 455 (M+H)⁺(free)

Preparation 99

3-Bromo-1,1-diphenyl-2-propanone (12.7 g) and N,N-diisopropylethylamine (15.7 ml) were added successively to a solution of (2S)-2-[(2-methoxybenzylamino)methyl]-pyrrolidine-1-carboxylic acid benzyl ester (15.6 g) in tetrahydrofuran (156 ml) at 0° C. After being stirred at room temperature for 2 hours, the mixture was poured into ice-water (100 ml) and extracted with ethyl acetate (100 ml×2). The extract was washed with brine, dried over magnesium sulfate and evaporated under reduced pressure. The residue was purified by column chromatography on silica gel using a mixed solvent of hexane and ethyl acetate (3:1). The fractions containing the objective compound were collected and evaporated under reduced pressure to give a colorless syrup of (2S)-2-[[N-(2-oxo-3,3-diphenylpropyl)-N-(2-methoxybenzyl)amino]methyl]pyrrolidine-1-carboxylic acid benzyl ester (1.51 g).

NMR (CDCl₃, δ): 1.30-2.00 (3H, m), 2.23-2.70 (2H, m), 3.11-3.93 (8H, m), 3.74 (3H, s), 5.06 (2H, m), 5.36 (1H, m), 6.82-7.31 (19H, m)

MASS (APCI): 563 (M+H)⁺

Preparation 100

The following compound was obtained according to a similar manner to that of Preparation 99.

### Benzyl (2S, 4R)-2-[[N-benzyl-N-(2-oxo-3,3-diphenylpropyl)amino]methyl]-4-[[tert-butyl(dimethyl)silyl]oxy]-1-pyrrolidinecarboxylate

NMR (CDCl₃, δ): 0.13 (6H, s), 0.82 (9H, s), 1.60-4.20 (12H, m), 5.00-5.20 (3H, m), 7.16-7.35 (20H, m)

MASS (APCI): 685 (M+Na), 663 (M+H)⁺, 505, 455, 415, 356

Preparation 101

A solution of (2S, 4R)-2-[[N-benzyl-N-(2-oxo-3,3-diphenylpropyl)amino]methyl]-4-[(tert-butyldimethylsilyl)-oxy]-1-pyrrolidinecarboxylate (4.82 g) and acetic acid (0.87 g) in methanol (100 ml) was hydrogenated over 10% palladium-charcoal (50% wet, 1.0 g) at room temperature under 2-3 atoms for 15 hours. After removal of the catalyst by filtration, the filtrate was evaporated under reduced pressure to give bis(acetic acid) salt of (7R, 8aS)-4-benzhydryl-7-[(tert-butyldimethylsilyl)oxy]octahydropyrrolo-[1,2-a]pyrazine (4.05 g) as a syrup.

IR (KBr): 3400, 1648, 1504 cm⁻¹

NMR (CDCl₃, δ): −0.20-−0.11 (6H, m), 0.74-0.81 (9H, m), 2.03 (6H, s), 1.60-1.80 (2H, m), 2.00-4.70 (10H, m), 7.16-7.35 (10H, m)

MASS (APCI): 423 (M+H)⁺(free)

Preparation 102

Di-tert-butyl dicarbonate (4.4 g) was added to an ice-cooled mixture of bis(acetic acid) salt of (7R, 8aS)-4-benzhydryl-7-[(tert-butyldimethylsilyl)oxy]octahydropyrrolo-[1,2-a]pyrazine (7.6 g) and triethylamine (4.9 ml) in dichloromethane (200 ml). After being stirred at the same temperature for 3 hours, the reaction mixture was washed with water and brine successively, dried over magnesium sulfate and evaporated under reduced pressure. The residue was purified by column chromatography on silica gel using a mixed solvent of hexane and ethyl acetate (4:1). The eluting fractions were collected and evaporated under reduced pressure to give colorless oil of tert-butyl (7R, 8aS)-4-benzhydryl-7-[(tert-butyldimethylsilyl)oxy]-hexahydropyrrolo[1,2-a]pyrazine-2(1H)-carboxylate (6.9 g). This compound (6.88 g) was dissolved into 1 M tetrabutylammonium floride in tetrahydrofuran (65 ml). After being stirred for 3 hours at room temperature, the reaction mixture was poured into water, the whole was extracted with ethyl acetate. The extract was washed with brine, dried over magnesium sulfate and evaporated under reduced pressure. The syrup was purified by column chromatography on silica gel using a mixed solvent of hexane and ethyl acetate (4:1). The later eluting fractions were collected and evaporated under reduced pressure to give colorless oil of tert-butyl (4R, 7R, 8aS)-4-benzhydryl-7-hydroxyhexahydropyrrolo[1,2-a]pyrazine-2(1H)-carboxylate (1.3 g).

IR (neat): 1695, 1504 cm⁻¹

NMR (CDCl₃, δ): 1.43 (9H, s), 1.31-1.74 (3H, m), 2.20-2.75 (3H, m), 1.93 (1H, dd, J=4.2 and 9.9 Hz), 3.08 (1H, dd, J=6.1 and 9.9 Hz), 3.30-3.40 (1H, m), 3.60-3.70 (1H, m), 3.78 (1H, br s), 3.94 (1H, d, J=9.0 Hz), 4.15-4.19 (1H, m), 7.13-7.45 (10H, m)

MASS (APCI): 409 (M+H)⁺(free)

The earlier eluting fractions were collected and evaporated under reduced pressure to give colorless oil of tert-butyl (4S, 7R, 8aS)-4-benzhydryl-7-hydroxyhexahydropyrrolo[1,2-a]pyrazine-2(1H)-carboxylate (1.5 g).

NMR (CDCl₃, δ): 1.32 (9H, s), 1.50-2.00 (3H, m), 2.40-2.55 (2H, m), 3.00-3.10 (2H, m), 3.40-4.05 (5H, m), 4.30 (1H, d, J=11.2 Hz), 7.15-7.45 (10H, m)

MASS (APCI): 409 (M+H)⁺(free)

Preparation 103

Triphenylphosphine (860 mg), acetic acid (159 mg) and diisopropyl azodicarboxylate were added successively into a solution of tert-butyl (4R, 7R, 8aS)-4-benzhydryl-7-hydroxyhexahydropyrrolo[1,2-a]pyrazine-2(1H)-carboxylate (670 mg) in tetrahydrofuran (10 ml) at room temperature. After being stirred for 1 hour at room temperature, the reaction mixture was poured into aqueous saturated sodium bicarbonate. The whole was extracted with ethyl acetate. The extract was washed with brine, dried over magnesium sulfate and evaporated under reduced pressure. The resulting residue was purified by silica gel column chromatography with a mixture of hexane and ethyl acetate (2:1-3:2) as an eluent to give tert-butyl (4R, 7S, 8aS)-7-acetoxy-4-benzhydrylhexahydropyrrolo[1,2-a]pyrazine-2(1H)-carboxylate.

NMR (CDCl₃, δ): 1.30-1.43 (11H, m), 2.01-2.04 (3H, m), 2.08-2.79 (6H, m), 3.12 (1H, m), 3.77-4.10 (2H, m), 4.89-5.01 (1H, m), 6.71-7.42 (10H, m)

MASS (APCI): 451 (M+H)⁺

Preparation 104

Sodium methoxide in methanol (5M, 27 μl) was added into a solution of tert-butyl (4R, 7S, 8aS)-7-acetoxy-4-benzhydrylhexahydropyrrolo[1,2-a]pyrazine-2(1H)-carboxylate (628 mg) in methanol (10 ml) at room temperature. After being stirred for 1 hour at the same temperature, the reaction mixture was poured into water (10 ml). The whole was extracted with ethyl acetate. The extract was washed with brine, dried over magnesium sulfate and evaporated under reduced pressure. The resulting residue was purified by silica gel column chromatography with a mixture of hexane and ethyl acetate (1:1) as an eluent to give tert-butyl (4R, 7S, 8aS)-4-benzhydryl-7-hydroxyhexahydropyrrolo[1,2-a]-pyrazine-2(1H)-carboxylate (521 mg).

NMR (CDCl₃, δ): 1.20-1.38 (11H, m), 1.80-1.98 (2H, m), 2.14-2.33 (2H, m), 2.43-2.74 (3H, m), 3.10 (1H, br), 3.73 (1H, br), 4.04-4.09 (2H, m), 7.14-7.41 (10H, m)

MASS (APCI): 409 (M+H)⁺

Preparation 105

Methanesulfonyl chloride (0.18 ml) was added dropwise to an ice-cooled solution of tert-butyl (4R, 7R, 8aS)-4-benzhydryl-7-hydroxyhexahydropyrrolo[1,2-a]pyrazine-2(1H)-carboxylate (0.78 g) and triethylamine (0.53 ml) in dichloromethane. After being stirred for 3 hours at the same temperature, the mixture was washed with aqueous saturated sodium bicarbonate, dried over magnesium sulfate and concentrated under reduced pressure. The syrup obtained by above procedure and sodium azide (126 mg) was dissolved into dimethylsulfoxide (5 ml). The whole was stirred at 75° C. for 15 hours. The mixture was poured into water and extracted with ethyl acetate. The extract was washed with brine, dried over magnesium sulfate and concentrated under reduced pressure. The syrup was purified by column chromatography on silica gel using a mixed solvent of hexane and ethyl acetate (30:1). The fractions containing the objective compound were collected to give (4R, 7S, 8aS)-4-benzhydryl-2-(tert-butoxycarbonyl)octahydropyrrolo[1,2-a]pyrazine-7-azide (0.70 mg).

NMR (CDCl₃, δ): 1.30-1.40 (2H, m), 1.38 (9H, s), 1.98-2.06 (1H, m), 2.15-2.27 (2H, m), 2.31-2.65 (2H, m), 2.78 (1H, d, J=8.6 Hz), 3.00-3.20 (1H, m), 3.63-3.72 (2H, m), 4.04 (1H, d, J=8.7 Hz), 7.13-7.43 (10H, m)

MASS (APCI): 434 (M+H)⁺(free)

Preparation 106

10% Palladium-charcoal (50% wet, 40 mg) and 0.1N hydrochloric acid (0.1 ml) were added into a solution of tert-butyl (4R, 7R, 8aS)-7-azido-4-benzhydrylhexahydropyrrolo-[1,2-a]pyrazine-2(1H)-carboxylate (200 mg) in methanol (2.5 ml) at room temperature. The mixture was hydrogenated at room temperature under atmospheric pressure for 4 hours. The palladium was filtered and washed with methanol. The filtrate and washings were combined and concentrated in vacuo. The resulting residue was partitioned between aqueous sodium bicarbonate and ethyl acetate. The organic layer was separated, dried over magnesium sulfate and evaporated under reduced pressure. The resulting residue was purified by silica gel column chromatography with a mixture of dichloromethane and methanol (15:1) as an eluent. The fractions containing the objective compound were collected to give tert-butyl (4R, 7R, 8aS)-7-amino-4-benzhydrylhexahydropyrrolo[1,2-a]pyrazine-2(1H)-carboxylate (193 mg).

NMR (CDCl₃, δ): 1.22-1.65 (15H, m), 2.30-2.51 (3H, m), 3.00-3.40 (2H, m), 3.68-4.10 (3H, m), 7.13-7.42 (10H, m)

MASS (APCI): 408 (M+H)⁺

Preparation 107

The following compound was obtained according to a similar manner to that of Preparation 105.

### tert-Butyl (4R, 7R, 8aS)-7-azido-4-benzhydrylhexahydropyrrolo[1,2-a]pyrazine-2(1H)-carboxylate

NMR (CDCl₃, δ): 1.38 (9H, s), 1.64-1.91 (3H, m), 2.40-2.60 (3H, m), 3.05-3.24 (2H, m), 3.82-4.18 (4H, m), 7.15-7.41 (10H, m)

MASS (APCI): 433 (M+H)⁺

Preparation 108

The following compound was obtained according to a similar manner to that of Preparation 109 from tert-butyl (4R, 7R, 8aS)-7-amino-4-benzhydrylhexahydropyrrolo[1,2-a]pyrazine-2(1H)-carboxylate.

### tert-Butyl (4R, 7R, 8aS)-7-(acetylamino)-4-benzhydrylhexahydropyrrolo[1,2-a]pyrazine-2(1H)-carboxylate

NMR (CDCl₃, δ): 1.37 (9H, s), 1.71 (5H, m), 2.03 (1H, dd, J=3.3, 7.3 Hz), 2.46-2.61 (2H, m), 2.94-3.07 (2H, m), 3.54-3.90 (4H, m), 4.21-4.28 (1H, m), 5.13-5.17 (1H, m), 7.15-7.42 (10H, m)

MASS (APCI): 450 (M+H)⁺

Preparation 109

Acetic anhydride (25.3 μl) was added to an ice cooled solution of tert-butyl (4R, 7S, 8aS)-7-amino-4-benzhydrylhexahydropyrrolo[1,2-a]pyrazine-2(1H)-carboxylate (0.1 g) and pyridine (0.096 ml) in dichloromethane (1 ml). After being stirred for 2 hours at the same temperature, the mixture was poured into aqueous sodium bicarbonate and extracted with dichloromethane. The organic layer was separated, dried over magnesium sulfate, concentrated under reduced pressure. The syrup was purified by column chromatography on silica gel using a mixed solvent of hexane and ethyl acetate (4:1). The fractions containing the objective compound were collected to give tert-butyl (4R, 7S, 8aS)-7-(acetylamino)-4-benzhydrylhexahydropyrrolo-[1,2-a]pyrazine-2(1H)-carboxylate (110 mg) as a syrup.

MASS (APCI): 450 (M+H)⁺

### EXAMPLE 1

To a mixture of 2-[(6R, 9aR)-6-benzhydryloctahydro-2H-pyrazino[1,2-a]pyrazin-2-yl]-2-oxoethanol (74 mg) and 2,4,6-trimethoxy-5-pyrimidinecarbaldehyde (40.1 mg) in dichloromethane (3 ml) was added sodium triacetoxyborohydride (85.8 mg) at room temperature, and then the whole was stirred at the same temperature for 15 hours. The reaction was quenched with saturated aqueous sodium bicarbonate, and the mixture was extracted with dichloromethane. The extract was dried over sodium sulfate, and concentrated under reduced pressure. The residue was purified by preparative TLC eluting with a mixture of dichloromethane and methanol (12:1) to give 88 mg of the product. The product was treated with 4N hydrogen chloride in ethyl acetate (0.081 ml). It was evaporated under reduced pressure to give 2-[(6R, 9aR)-6-benzhydryl-8-[(2,4,6-trimethoxy-5-pyrimidinyl)methyl]octahydro-2H-pyrazino[1,2-a]pyrazin-2-yl]-2-oxoethanol dihydrochloride (87 mg) as a colorless powder.

mp: 155-158° C. (decomp)

IR (KBr): 3435, 1658, 1601, 1576, 1379, 1232, 1146 cm⁻¹

NMR (DMSO-d₆, δ): 3.80 (6H, s), 3.91 (3H, s), 2.20-4.80 (18H, m), 7.10-7.50 (10H, m)

MASS (API-ES): 548 (M+H)⁺(free)

### EXAMPLE 2

The following compounds were obtained according to a similar manner to that of Example 1.

### (1) 2-[(6R, 9aR)-6-Benzhydryl-8-[(2-isopropoxy-4,6-dimethoxy-5-pyrimidinyl)methyl]octahydro-2H-pyrazino[1,2-a]pyrazin-2-yl]-2-oxoethanol Dihydrochloride

NMR (DMSO-d₆, δ): 1.33 (6H, d, J=6.1 Hz), 3.78 (6H, s), 2.20-4.45 (18H, m), 5.17 (1H, m), 7.10-7.45 (10H, m)

MASS (API-ES): 576 (M+H)⁺(free)

### (2) 2-[(6R, 9aR)-6-Benzhydryl-8-[(4,6-dimethoxy-2-methyl-5-pyrimidinyl)methyl]octahydro-2H-pyrazino[1,2-a]pyrazin-2-yl]-2-oxoethanol Dihydrochloride

mp: 129-132° C.

IR (KBr): 3402, 1651, 1572, 1454, 1271, 1146 cm⁻¹

NMR (DMSO-d₆, δ): 2.46 (3H, s), 3.81 (6H, s), 2.05-4.35 (18H, m), 7.10-7.45 (10H, m)

MASS (API-ES): 532 (M+H)⁺(free)

### (3) 2-[(6R, 9aR)-6-Benzhydryl-8-[(2,4,6-triethoxy-5-pyrimidinyl)methyl]octahydro-2H-pyrazino[1,2-a]pyrazin-2-yl]-2-oxoethanol Dihydrochloride

NMR (DMSO-d₆, δ): 1.14-1.40 (9H, m), 4.15-4.40 (6H, m), 2.20-4.50 (18H, m), 7.10-7.45 (10H, m)

MASS (API-ES): 5.90 (M+H)⁺(free)

### (4) 2-[(6R, 9aR)-6-Benzhydryl-8-[(4-isopropoxy-2,6-dimethoxy-5-pyrimidinyl)methyl]octahydro-2H-pyrazino[1,2-a]pyrazin-2-yl]-2-oxoethanol Dihydrochloride

mp: 145-148° C. (decomp)

IR (KBr): 3400, 1657, 1599, 1572, 1381, 1149 cm⁻¹

NMR (DMSO-d₆, δ): 1.13-1.25 (6H, m), 3.89 (3H, s), 2.20-4.70 (21H, m), 5.16 (1H, m), 7.10-7.50 (10H, m)

MASS (API-ES): 576 (M+H)⁺(free)

### (5) 2-[(6R, 9aR)-6-Benzhydryl-8-[(4-chloro-2,6-dimethoxy-5-pyrimidinyl)methyl]octahydro-2H-pyrazino[1,2-a]pyrazin-2-yl]-2-oxoethanol Dihydrochloride

mp: 98-101° C. (decomp)

IR (KBr): 3402, 1662, 1577, 1375, 1234 cm⁻¹

NMR (DMSO-d₆, δ): 3.94 (3H, s), 2.20-4.80 (21H, m), 7.10-7.60 (10H, m)

MASS (API-ES): 552 (M)⁺(free)

### (6) 2-[(6R, 9aR)-6-Benzhydryl-8-[(2-ethoxy-4,6-dimethoxy-5-pyrimidinyl)methyl]octahydro-2H-pyrazino[1,2-a]pyrazin-2-yl]-2-oxoethanol Dihydrochloride

NMR (DMSO-d₆, δ): 1.33 (3H, t, J=7.0 Hz), 3.79 (6H, s), 4.36 (2H, q, J=7.0 Hz), 2.20-4.45 (18H, m), 7.10-7.45 (10H, m)

MASS (API-ES): 562 (M+H)⁺(free)

### (7) 2-[(6R, 9aR)-6-Benzhydryl-8-[(2-chloro-4,6-dimethoxy-5-pyrimidinyl)methyl]octahydro-2H-pyrazino[1,2-a]pyrazin-2-yl]-2-oxoethanol Dihydrochloride

mp: 150-155° C. (decomp)

IR (KBr): 3402, 1662, 1595, 1537, 1383, 1234 cm⁻¹

NMR (DMSO-d₆, δ): 3.83 (6H, s), 2.10-4.90 (18H, m), 7.10-7.60 (10H, m)

MASS (API-ES): 552 (M)⁺(free)

### (8) 2-[(6R, 9aR)-6-Benzhydryl-8-[[4-(2,2-difluoroethoxy)-2,6-dimethoxy-5-pyrimidinyl]methyl]octahydro-2H-pyrazino[1,2-a]pyrazin-2-yl]-2-oxoethanol Dihydrochloride

mp: 143-146° C.

IR (KBr): 3435, 1658, 1608, 1576, 1383, 1232, 1080 cm⁻¹

NMR (DMSO-d₆, δ): 3.92 (3H, s), 2.10-4.60 (21H, m), 4.40-4.70 (2H, m), 6.05-6.70 (1H, m), 7.10-7.50 (10H, m)

MASS (API-ES): 598 (M+H)⁺(free)

### (9) 2-[(6R, 9aR)-6-Benzhydryl-8-[(2,4-dimethoxy-6-methyl-5-pyrimidinyl)methyl]octahydro-2H-pyrazino[1,2-a]pyrazin-2-yl]-2-oxoethanol Dihydrochloride

mp: 174-177° C.

IR (KBr): 3402, 1651, 1454, 1375 cm⁻¹

NMR (DMSO-d₆, δ): 2.42 (3H, s), 3.89 (3H, s), 2.20-4.60 (21H, m), 7.10-7.50 (10H, m)

MASS (API-ES): 532 (M+H)⁺(free)

### (10) 2-[(6R, 9aR)-6-Benzhydryl-8-[(4-isobutyl-2,6-dimethoxy-5-pyrimidinyl)methyl]octahydro-2H-pyrazino[1,2-a]pyrazin-2-yl]-2-oxoethanol Dihydrochloride

NMR (CDCl₃, δ) (free form): 0.87 (3H, d, J=5.12 Hz), 0.90 (3H, d, J=5.16 Hz), 1.71-3.35 (16H, m), 3.55 (1H, br q, J=3.38 Hz), 3.77 and 3.81 (total 3H, each s), 3.94 (3H, s), 4.03-4.17 (4H, m), 7.12-7.29 (10H, m)

MASS (API-ES): 574 (M+H)⁺

### (11) 2-[(6R, 9aR)-6-Benzhydryl-8-[(4-ethoxy-2,6-dimethoxy-5-pyrimidinyl)methyl]octahydro-2H-pyrazino[1,2-a]pyrazin-2-yl]-2-oxoethanol Dihydrochloride

mp: 150-152° C. (decomp)

IR (KBr): 3402, 1658, 1601, 1576, 1381, 1232, 1147 cm⁻¹

NMR (DMSO-d₆, δ): 1.21 (3H, t, J=7.1 Hz), 3.90 (3H, s), 4.25 (2H, q, J=7.1 Hz), 2.10-4.50 (21H, m), 7.10-7.50 (10H, m)

MASS (API-ES): 562 (M+H)⁺(free)

### (12) 2-[(6R, 9aR)-6-Benzhydryl-8-[[4-(cyclobutyloxy)-2,6-dimethoxy-5-pyrimidinyl]methyl]octahydro-2H-pyrazino[1,2-a]pyrazin-2-yl]-2-oxoethanol Dihydrochloride

mp: 141-145° C.

IR (KBr): 3425, 2949, 1651, 1597, 1576, 1381, 1230, 1149 cm⁻¹

NMR (DMSO-d₆, δ): 3.88 (3H, s), 1.50-4.40 (27H, m), 5.01 (1H, m), 7.10-7.50 (10H, m)

MASS (API-ES): 588 (M+H)⁺(free)

### (13) 2-[(6R, 9aR)-6-Benzhydryl-8-[(4,6-diethoxy-2-methoxy-5-pyrimidinyl)methyl]octahydro-2H-pyrazino[1,2-a]pyrazin-2-yl]-2-oxoethanol Dihydrochloride

IR (KBr): 3276, 1645, 1568, 1146, 1084 cm⁻¹

NMR (DMSO-d₆+D₂O, δ): 1.22 (6H, t, J=7.0 Hz), 1.85-4.55 (30H, m), 7.10-7.55 (10H, m)

MASS (ES): 598.3 (M+Na)⁺(free), 576.4 (M+H)⁺(free).

### (14) (4R, 9aR)-4-Benzhydryl-2-[(4-isopropoxy-2,6-dimethoxy-5-pyrimidinyl)methyl]-8-(5-isoxazolylcarbonyl)octahydro-2H-pyrazino[1,2-a]pyrazine Dihydrochloride

NMR (DMSO-d₆, δ): 1.05-1.35 (6H, m), 3.79 (3H, s), 3.89 (3H, s), 2.20-4.60 (15H, m), 5.15 (1H, m), 6.93 (1H, d, J=1.8 Hz), 7.10-7.45 (10H, m), 8.76 (1H, d, J=1.8 Hz)

MASS (API-ES): 613 (M+H)⁺(free)

### (15) (4R, 9aR)-4-Benzhydryl-2-[(2-cyclopropyl-4-isopropoxy-6-methoxy-5-pyrimidinyl)methyl]-8-(methoxyacetyl)-octahydro-2H-pyrazino[1,2-a]pyrazine Dihydrochloride

NMR (DMSO-d₆, δ): 0.95-1.22 (10H, m), 1.99 (1H, m), 2.15-4.50 (23H, m), 5.14 (1H, m), 7.10-7.45 (10H, m)

MASS (API-ES): 600 (M+H)⁺(free)

### (16) 3-[(6R, 9aR)-6-Benzhydryl-8-[(2-cyclopropyl-4-ethoxy-6-methoxy-5-pyrimidinyl)methyl]octahydro-2H-pyrazino[1,2-a]pyrazin-2-yl]-3-oxo-1-propanol Dihydrochloride

NMR (DMSO-d₆, δ): 1.01-1.05 (4H, m), 1.20 (3H, t, J=7.0 Hz), 2.01 (1H, m), 3.80 (3H, s), 4.22 (2H, q, J=7.0 Hz), 2.10-4.50 (20H, m), 7.10-7.45 (10H, m)

MASS (API-ES): 586 (M+H)⁺(free)

### (17) (4R, 9aR)-4-Benzhydryl-2-[(2-cyclopropyl-4-ethoxy-6-methoxy-5-pyrimidinyl)methyl]-8-(methoxyacetyl)-octahydro-2H-pyrazino[1,2-a]pyrazine Dihydrochloride

NMR (DMSO-d₆, δ): 1.01 (4H, m), 1.20 (3H, t, J=7.0 Hz), 2.01 (1H, m), 3.78 (3H, s), 4.22 (2H, q, J=7.0 Hz), 2.15-4.50 (20H, m), 7.10-7.50 (10H, m)

MASS (API-ES): 586 (M+H)⁺(free)

### (18) 2-[(6R, 9aR)-6-[Bis(4-fluorophenyl)methyl]-8-[(4-isopropoxy-2,6-dimethoxy-5-pyrimidinyl)methyl]-octahydro-2H-pyrazino[1,2-a]pyrazin-2-yl]-2-oxoethanol Dihydrochloride

NMR (DMSO-d₆, δ): 1.15-1.24 (3H, m), 2.10-4.45 (23H, m), 5.10-5.25 (1H, m), 7.09-7.40 (8H, m)

MASS (ESI): 612.1 (M+H)⁺(free)

### (19) 2-[(6R, 9aR)-6-[Bis(4-methylphenyl)methyl]-8-[(4-isopropoxy-2,6-dimethoxy-5-pyrimidinyl)methyl]-octahydro-2H-pyrazino[1,2-a]pyrazin-2-yl]-2-oxoethanol Dihydrochloride

NMR (DMSO-d₆, δ): 1.15-1.24 (6H, m), 2.23 (6H, s), 2.20-4.20 (23H, m), 5.10-5.20 (1H, m), 7.07-7.24 (8H, m)

MASS (ESI): 604.35 (M+H)⁺(free)

### EXAMPLE 3

To a mixture of (6R, 9aR)-2-acetyl-6-benzhydryloctahydro-2H-pyrazino[1,2-a]pyrazine dihydrochloride (65 mg) and N,N-diisopropylethylamine (49.7 mg) in dichloromethane (2 ml) were added 2,4,6-trimethoxy-5-pyrimidinecarbaldehyde (30.5 mg) and sodium triacetoxyborohydride (65.2 mg) successively at room temperature, and then the whole was stirred at the same temperature for 15 hours. The reaction mixture was quenched with saturated aqueous sodium bicarbonate, and extracted with dichloromethane. The extract was dried over sodium sulfate, and concentrated under reduced pressure. The residue was purified by preparative TLC eluting with a mixture of dichloromethane and methanol (12:1) to give 64 mg of the product. The product was treated with 4N hydrogen chloride in ethyl acetate (0.061 ml). It was evaporated under reduced pressure to give (4R, 9aR)-8-acetyl-4-benzhydryl-2-[(2,4,6-trimethoxy-5-pyrimidinyl)methyl]-octahydro-2H-pyrazino[1,2-a]pyrazine dihydrochloride (64 mg) as a colorless powder.

IR (Neat): 3203, 1635, 1597, 1577, 1458, 1381, 1234, 1147 cm⁻¹

NMR (DMSO-d₆, δ): 1.96 (3H, s), 3.80 (6H, s), 3.91 (3H, s), 2.20-4.80 (15H, m), 7.10-7.50 (10H, m)

MASS (API): 531 (M)⁺(free)

### EXAMPLE 4

The following compounds were obtained according to a similar manner to that of Example 3.

### (1) 2-[(6R, 9aR)-6-Benzhydryl-8-[(2,4-diethoxy-6-methoxy-5-pyrimidinyl)methyl]octahydro-2H-pyrazino[1,2-a]pyrazin-2-yl]-2-oxoethanol Dihydrochloride

NMR (DMSO-d₆, δ): 1.21 (3H, t, J=6.8 Hz), 1.33 (3H, t, J=7.0 Hz), 2.30-4.20 (20H, m), 4.23 (2H, q, J=7.0 Hz), 4.33 (2H, q, J=6.8 Hz), 7.19-7.41 (10H, m)

MASS (API-ES): 598 (M+Na)⁺, 576 (M+H)⁺

### (2) 2-[(6R, 9aR)-6-Benzhydryl-8-[(2-ethoxy-4-isopropoxy-6-methoxy-5-pyrimidinyl)methyl]octahydro-2H-pyrazino-[1,2-a]pyrazin-2-yl]-2-oxoethanol Dihydrochloride

NMR (DMSO-d₆, δ): 1.03-1.30 (10H, m), 2.30-4.20 (19H, m), 4.33 (2H, d, J=7.0 Hz), 5.14 (1H, sept, J=6.0 Hz), 7.17-7.41 (10H, m)

MASS (API-ES): 590 (M+H)⁺

### (3) (4R, 9aR)-4-Benzhydryl-2-[(2-ethoxy-4-isopropoxy-6-methoxy-5-pyrimidinyl)methyl]-8-(methoxyacetyl)-octahydro-2H-pyrazino[1,2-a]pyrazine Dihydrochloride

NMR (DMSO-d₆, δ): 1.03-1.20 (6H, m), 1.33 (3H, t, J=7.0 Hz), 2.30-4.20 (23H, m), 4.33 (2H, d, J=7.0 Hz), 5.10-5.20 (1H, m), 7.19-7.39 (˜10H, m), 10.60-11.30 (2H, m)

MASS (API-ES): 604 (M+H)⁺

### (4) N-[(4R, 7R, 8aS)-4-Benzhydryl-2-[(4-isopropoxy-2,6-dimethoxy-5-pyrimidinyl)methyl]octahydropyrrolo-[1,2-a]pyrazin-7-yl]acetamide Dihydrochloride

NMR (DMSO-d₆, δ): 1.14-1.23 (6H, m), 1.68 (3H, s), 3.74 (3H, s), 3.89 (3H, s), 1.60-4.80 (14H, m), 5.15 (1H, m), 7.10-7.50 (10H, m), 8.00 (1H, br)

MASS (API-ES): 560 (M+H)⁺(free)

### (5) (4R, 9aR)-4-Benzhydryl-2-[(4-isopropoxy-2,6-dimethoxy-5-pyrimidinyl)methyl]-8-(3-pyridylcarbonyl)octahydro-2H-pyrazino[1,2-a]pyrazine trihydrochloride

mp: 143-147° C.

IR (KBr): 3433, 1647, 1595, 1576, 1232, 1147 cm⁻¹

NMR (DMSO-d₆, δ): 1.13-1.24 (6H, m), 3.80 (3H, s), 3.90 (3H, s), 2.25-4.70 (15H, m), 5.16 (1H, m), 7.10-7.45 (10H, m), 7.77 (1H, m), 8.14 (1H, m), 8.80 (2H, m)

MASS (API-ES): 623 (M+H)⁺(free)

### (6) (4R, 9aR)-4-Benzhydryl-8-(2-pyridylcarbonyl)-2-[(2,4,6-trimethoxy-5-pyrimidinyl)methyl]octahydro-2H-pyrazino[1,2-a]pyrazine Trihydrochloride

IR (KBr): 3404, 1635, 1597, 1577, 1379, 1146 cm⁻¹

NMR (DMSO-d₆, δ): 3.78 (6H, s), 3.92 (3H, s), 2.10-4.50 (15H, m), 7.10-7.45 (10H, m), 7.50-7.65 (2H, m), 7.95 (1H, m), 8.56 (1H, m)

MASS (API-ES): 595 (M+H)⁺(free)

### (7) 3-[(6R, 9aR)-6-Benzhydryl-8-[(2-ethoxy-4-isopropoxy-6-methoxy-5-pyrimidinyl)methyl]octahydro-2H-pyrazino[1,2-a]pyrazin-2-yl]-3-oxo-1-propanol Dihydrochloride

NMR (DMSO-d₆, δ): 1.14-1.39 (6H, m), 1.32 (3H, t, J=7.0 Hz), 2.20-4.00 (24H, m), 4.33 (2H, q, J=7.0 Hz), 5.10-5.16 (1H, m), 7.19-7.43 (10H, m)

MASS (API-ES): 626 (M+Na)⁺, 604 (M+H)⁺

### (8) (4R, 9aR)-4-Benzhydryl-8-(3-pyridylcarbonyl)-2-[(2,4,6-trimethoxy-5-pyrimidinyl)methyl]octahydro-2H-pyrazino[1,2-a]pyrazine Trihydrochloride

NMR (DMSO-d₆, δ): 3.79 (6H, s), 3.91 (3H, s), 2.25-4.60 (15H, m), 7.10-7.50 (10H, m), 7.82 (1H, m), 8.20 (1H, d, J=7.6 Hz), 8.82 (2H, m)

MASS (API-ES): 595 (M+H)⁺(free)

### (9) (4R, 9aR)-4-Benzhydryl-2-[(2-ethoxy-4-isopropoxy-6-methoxy-5-pyrimidinyl)methyl]-8-(3-methoxypropanoyl)-octahydro-2H-pyrazino[1,2-a]pyrazine Dihydrochloride

NMR (DMSO-d₆, δ): 1.16-1.23 (6H, m), 1.31-1.34 (3H, m), 2.30-4.60 (27H, m), 5.13-5.15 (1H, m), 7.19-7.42 (10H, m), 11.00-12.00 (2H, m)

MASS (API-ES): 640 (M+Na)⁺, 618 (M+H)⁺(free)

### (10) (4R, 9aR)-4-Benzhydryl-8-(morpholinocarbonyl)-2-[(2,4,6-trimethoxy-5-pyrimidinyl)methyl]octahydro-2H-pyrazino[1,2-a]pyrazine Dihydrochloride

mp: 139-143° C.

IR (KBr): 3437, 1645, 1603, 1576, 1464, 1379, 1234 cm⁻¹

NMR (DMSO-d₆, δ): 3.79 (6H, s), 3.91 (3H, s), 2.15-4.60 (23H, m), 7.10-7.50 (10H, m)

MASS (API-ES): 603 (M+H)⁺(free)

### (11) (4R, 9aR)-4-Benzhydryl-2-[(4-isopropoxy-2,6-dimethoxy-5-pyrimidinyl)methyl]-8-morpholinocarbonyl)octahydro-2H-pyrazino[1,2-a]pyrazine Dihydrochloride

mp: 136-139° C.

IR (KBr): 3437, 1645, 1697, 1576, 1460, 1425, 1379, 1232, 1149, 1109 cm⁻¹

NMR (DMSO-d₆, δ): 1.10-1.30 (6H, m), 3.76 (3H, s), 3.89 (3H, s), 2.20-4.80 (23H, m), 5.17 (1H, m), 7.10-7.50 (10H, m)

MASS (API-ES): 631 (M+H)⁺(free)

### (12) 2-[(6R, 9aR)-6-Benzhydryl-8-[(2-isopropyl-4,6-dimethoxy-5-pyrimidinyl)methyl]octahydro-2H-pyrazino[1,2-a]pyrazin-2-yl]-2-oxoethanol Dihydrochloride

NMR (CDCl₃, δ): 1.26 (6H, d, J=6.6 Hz), 2.95 (1H, sept, J=6.6 Hz), 3.82 (6H, s), 2.20-4.60 (18H, m), 7.10-7.75 (10H, m)

MASS (API-ES): 560 (M+H)⁺(free)

### (13) 2-[(6R, 9aR)-6-Benzhydryl-8-[(2-cyclopropyl-4,6-diethoxy-5-pyrimidinyl)methyl]octahydro-2H-pyrazino[1,2-a]pyrazin-2-yl]-2-oxoethanol dihydrochoride

NMR (DMSO-d₆, δ): 0.80-1.30 (10H, m), 1.91-2.06 (1H, m), 2.20-4.60 (21H, m), 7.19-7.41 (10H, m)

MASS (API-ES): 608 (M+Na)⁺, 586 (M+H)⁺(free)

### (14) 2-[(6R, 9aR)-6-Benzhydryl-8-[(2-cyclopropyl-4,6-dimethoxy-5-pyrimidinyl)methyl]octahydro-2H-pyrazino[1,2-a]pyrazin-2-yl]-2-oxoethanol Dihydrochloride

NMR (DMSO-d₆, δ): 1.02-1.05 (4H, m), 1.91-2.15 (1H, m), 2.24-4.50 (24H, m), 7.20-7.40 (10H, m)

MASS (API-ES): 558 (M+H)⁺(free)

### (15) (4R, 9aR)-4-Benzhydryl-2-[(2-cyclopropyl-4-isopropoxy-6-methoxy-5-pyrimidinyl)methyl]-8-(3-methoxypropanoyl)octahydro-2H-pyrazino[1,2-a]pyrazine Dihydrochloride

NMR (DMSO-d₆, δ): 0.85-1.30 (10H, m), 2.01-2.06 (1H, m), 2.20-4.60 (25H, m), 5.15-5.20 (1H, m), 7.19-7.41 (10H, m)

MASS (API-ES): 636 (M+Na)⁺, 614 (M+H)⁺(free)

### (16) 2-[(6R, 9aR)-6-Benzhydryl-8-[(2-cyclopropyl-4-isopropoxy-6-methoxy-5-pyrimidinyl)methyl]octahydro-2H-pyrazino[1,2-a]pyrazin-2-yl]-2-oxoethanol Dihydrochloride

NMR (DMSO-d₆, δ): 1.03-1.30 (10H, m), 2.01-2.06 (1H, m), 2.30-5.20 (21H, m), 7.20-7.41 (10H, m), 10.00-13.00 (3H, m)

MASS (API-ES): 606 (M+Na)⁺, 586 (M+H)⁺(free)

### (17) 3-[(6R, 9aR)-6-Benzhydryl-8-[(2-cyclopropyl-4-isopropoxy-6-methoxy-5-pyrimidinyl)methyl]octahydro-2H-pyrazino[1,2-a]pyrazin-2-yl]-3-oxo-1-propanol Dihydrochloride

NMR (DMSO-d₆, δ): 1.01-1.25 (10H, m), 2.00-2.10 (1H, m), 2.20-4.60 (23H, m), 5.10-5.16 (1H, m), 7.19-7.43 (10H, m), 10.00-12.00 (1H, br s)

MASS (API-ES): 622 (M+Na)⁺, 600 (M+H)⁺(free)

### (18) 2-[(6R, 9aR)-6-Benzhydryl-8-[(2-cyclopropyl-4-ethoxy-6-methoxy-5-pyrimidinyl)methyl]octahydro-2H-pyrazino-[1,2-a]pyrazin-2-yl]-2-oxoethanol Dihydrochloride

NMR (DMSO-d₆, δ): 0.90-1.30 (7H, m), 1.95-2.10 (1H, m), 2.20-4.40 (32H, m), 7.18-7.41 (10H, m)

MASS (API-ES): 594 (M+Na)⁺, 572 (M+H)⁺(free)

### (19) (4R, 9aR)-4-Benzhydryl-2-[(2-cyclopropyl-4-ethoxy-6-methoxy-5-pyrimidinyl)methyl]-8-(3-methoxypropanoyl)-octahydro-2H-pyrazino[1,2-a]pyrazine Dihydrochloride

NMR (DMSO-d₆, δ): 0.85-1.10 (4H, m), 1.20 (3H, t, J=7.0 Hz), 1.95-2.05 (1H, m), 2.20-4.40 (25H, m), 4.23 (2H, q, J=7.0 Hz), 7.19-7.42 (10H, m)

MASS (API-ES): 622 (M+Na)⁺, 600 (M+H)⁺(free)

### (20) 5-[[(3R)-3-Benzhydryl-4-[(1-methyl-1H-pyrazol-4-yl)methyl]-1-piperazinyl]methyl]-2,4,6-trimethoxypyrimidine Dihydrochloride

NMR (CDCl₃, δ): 2.00-4.20 (26H, m), 7.20-7.50 (12H, m)

MASS (API): 529 (M+H)⁺(free)

### (21) 5-[[(3R)-3-Benzhydryl-4-[(1-methyl-1H-pyrazol-4-yl)methyl]-1-piperazinyl]methyl]-4-isopropoxy-2,6-dimethoxypyrimidine Dihydrochloride

NMR (DMSO-d₆, δ): 1.05-5.20 (30H, m), 6.80-7.50 (10H, m)

MASS (ESI): 557.4 (M+H)⁺(free)

### EXAMPLE 5

To a solution of 2-[(6R, 9aR)-6-benzhydryloctahydro-2H-pyrazino[1,2-a]pyrazin-2-yl]-2-oxoethanol (197 mg), potassium iodide (358 mg), and N,N-diisopropylethylamine (348 mg) in N,N-dimethylformamide (6 ml) was added a solution of 5-(1-chloroethyl)-2,4,6-trimethoxypyrimidine (376 mg) in dichloromethane (4.5 ml) at 0° C. and the mixture was stirred at the same temperature for 16 hours. The reaction mixture was diluted with ethyl acetate and washed with saturated aqueous sodium bicarbonate and brine, dried over magnesium sulfate, and concentrated under reduced pressure. The residue was purified by column chromatography on silica gel eluting first with ethyl acetate-hexane (1:2) and then with dichloromethane-methanol (20:1) to give 218 mg of a mixture of 2-[(6R, 9aR)-6-benzhydryl-8-[(1R)-1-(2,4,6-trimethoxy-5-pyrimidinyl)ethyl]octahydro-2H-pyrazino[1,2-a]pyrazin-2-yl]-2-oxoethanol and 2-[(6R, 9aR)-6-benzhydryl-8-[(1S)-(2,4,6-trimethoxy-5-pyrimidinyl)ethyl]octahydro-2H-pyrazino[1,2-a]pyrazin-2-yl]-2-oxoethanol. The mixture was purified by preparative TLC eluting with diethyl ether-methanol (10:1) to afford 71.4 mg (23.6%) of the less polar isomer and 70.5 mg (23.3%) of the more polar isomer. The less polar isomer; MASS (API-ES) 562 (M+H)⁺The more polar isomer; MASS (API-ES) 562 (M+H)⁺Treatments of the less and the more polar isomer with 4N hydrogene chloride in ethyl acetate (1 ml) and the volatile materials were evaporated under reduced pressure to give 59.6 mg and 29.6 mg of its corresponding hydrochloride of the amine derivatives above respectively.

### EXAMPLE 6

To a mixture of tert-butyl (2R)-2-benzhydryl-1-piperazinecarboxylate (150 mg) and 2,4,6-trimethoxy-5-pyrimidinecarbaldehyde (84 mg) in dichloromethane (3 ml) was added sodium triacetoxyborohydride (135 mg) at room temperature, and then the whole was stirred at the same temperature for 15 hours. The reaction was quenched with saturated aqueous sodium bicarbonate, and the mixture was extracted with dichloromethane. The extract was dried over sodium sulfate, and concentrated under reduced pressure. The residue was purified by column chromatography on silica gel using a mixed solvent of dichloromethane and methanol (20:1). The fractions containing the objective compound were collected and concentrated under reduced pressure to give an oil of tert-butyl (2R)-2-benzhydryl-4-[(2,4,6-trimethoxypyrimidin-5-yl)methyl]-1-piperazinecarboxylate.

NMR (CDCl₃, δ): 1.90-5.00 (10H, m), 3.92, 3.94, 3.98 (each 3H, s), 7.10-7.45 (10H, m)

MASS (API-ES): 535 (M+H)⁺

The oil was treated with 4N hydrogen chloride in ethyl acetate (2.5 ml), and the whole was evaporated under reduced pressure to give a foam of 5-[[(3R)-3-benzhydryl-1-piperazinyl]methyl]-2,4,6-trimethoxypyrimidine dihydrochloride (246 mg).

Preparation 110

To an ice-cooled solution of tert-butyl (4R, 9aS)-4-benzhydryloctahydro-2H-pyrazino[1,2-a]pyrazine-2-carboxylate (600 mg) and 2-pyrazinecarboxylic acid (201 mg) in dichloromethane (10 ml) were added 1-hydroxybenzotriazole (199 mg), 1-(3-dimethylaminopropyl)-3-ethylcarbodiimide hydrochloride (423 mg), and the whole was stirred at room temperature for 5 hours. The reaction mixture was poured into ice-water (15 ml), and the organic layer was washed with aqueous sodium hydrogen carbonate, and brine successively, then dried over sodium sulfate and evaporated under reduced pressure. The residue was purified by column chromatography on silica gel (20 g) using a mixed solvent of dichloromethane and methanol (35:1 to 25:1). The fractions containing the objective compound were collected and concentrated under reduced pressure to give tert-butyl (4R, 9aS)-4-benzhydryl-8-(2-pyrazinylcarbonyl)octahydro-2H-pyrazino[1,2-a]pyrazine-2-carboxylate as syrup (860 mg).

NMR (CDCl₃, δ): 1.20-1.55 (9H, m), 1.90-4.60 (13H, m), 7.10-7.40 (10H, m), 8.51 (1H, d, J=10.1 Hz), 8.62 (1H, d, J=8.1 Hz), 8.93 (1H, d, J=8.9 Hz)

MASS (API-ES): 536 (M+Na)⁺

Preparation 111

The following compounds were obtained according to a similar manner to that of Preparation 110.

### (1) tert-Butyl (4R, 9aS)-4-Benzhydryl-8-[(4-oxido-2-pyrazinyl)carbonyl]octahydro-2H-pyrazino[1,2-a]-pyrazine-2-carboxylate

NMR (CDCl₃, δ): 1.14-1.29 (9H, m), 2.00-2.25 (1H, m), 2.50-3.19 (6H, m), 3.57-3.95 (3H, m), 4.10-4.30 (3H, m), 7.19-7.30 (10H, m), 8.04-8.11 (1H, m), 8.33-8.40 (2H, m)

MASS (ES positive): 552 (M+Na)⁺

### (2) tert-Butyl (4R, 9aS)-8-[2-(acetylamino)-3-hydroxypropanoyl]-4-benzhydryloctahydro-2H-pyrazino[1,2-a]pyrazine-2-carboxylate

NMR (CDCl₃, δ): 1.33 (9H, s), 2.10-2.04 (4H, m), 2.25-2.55 (1H, m), 2.55-3.20 (6H, m), 3.25-3.85 (6H, m), 4.12-4.24 (2H, m), 4.74-4.94 (1H, m), 6.65-6.86 (1H, m), 7.18-7.29 (10H, m)

MASS (ES positive): 537 (M+H)⁺

### (3) tert-Butyl (4R, 9aS)-8-[2-(acetylamino)-3-hydroxybutanoyl]-4-benzhydryloctahydro-2H-pyrazino-[1,2-a]pyrazine-2-carboxylate

NMR (CDCl₃, δ): 1.09-1.17 (3H, m), 1.33 (9H, s), 1.90-2.11 (4H, m), 2.25-2.60 (1H, m), 2.60-3.25 (6H, m), 3.40-3.95 (3H, m), 3.95-4.17 (4H, m), 4.71 (1H, d, J=9.34 Hz), 6.26-6.34 (1H, m), 7.18-7.29 (10H, m)

MASS (ES positive): 551 (M+H)⁺

### (4) tert-Butyl (4R, 9aS)-4-benzhydryl-8-[(6-methoxy-2-pyrazinyl)carbonyl]octahydro-2H-pyrazino[1,2-a]-pyrazine-2-carboxylate

NMR (CDCl₃, δ): 1.14-1.39 (9H, m), 2.04-2.26 (1H, m), 2.55-3.12 (6H, m), 3.58-3.90 (3H, m), 3.84 and 3.97 (total 3H, each s), 4.10-4.23 (2H, m), 4.37-4.42 (1H, m), 7.16-7.29 (10H, m), 8.23 and 8.28 (total 1H, each s), 8.38 and 8.43 (total 1H, each s)

MASS (ES positive): 566 (M+Na)⁺

### (5) tert-Butyl (4R, 9aS)-4-benzhydryl-8-(1,4-dioxan-2-ylcarbonyl)octahydro-2H-pyrazino[1,2-a]pyrazine-2-carboxylate

NMR (CDCl₃, δ): 1.32 (9H, s), 1.95-2.10 (1H, m), 2.34-3.07 (7H, m), 3.49-3.95 (9H, m), 3.95-4.22 (3H, m), 7.21-7.29 (10H, m)

MASS (ES positive): 544 (M+Na)⁺

### (6) tert-Butyl (4R, 9aS)-4-benzhydryl-8-[(2R)-tetrahydro-2-furanylcarbonyl]octahydro-2H-pyrazino[1,2-a]pyrazine-2-carboxylate

NMR (CDCl₃, δ): 1.05-1.55 (9H, m), 1.80-3.20 (12H, m), 3.45-4.35 (7H, m), 4.40-4.65 (1H, m), 7.10-7.40 (10H, m)

MASS (API-ES, Pos): 506.2 (M+H)⁺, 528.3 (M+Na)⁺

### (7) tert-Butyl (4R, 9aS)-8-[(3-amino-2-pyrazinyl)carbonyl]-4-benzhydryloctahydro-2H-pyrazino[1,2-a]pyrazine-2-carboxylate

NMR (CDCl₃, δ): 1.10-1.55 (9H, m), 2.00-2.25 (1H, m), 2.45-4.45 (12H, m), 5.60-5.85 (2H, m), 7.10-7.40 (10H, m), 7.70-7.95 (1H, m), 7.95-8.10 (1H, m)

MASS (API-ES, Pos): 529.2 (M+H)⁺, 551.2 (M+Na)⁺

### (8) tert-Butyl (4R, 9aS)-4-benzhydryl-8-[(2S)-tetrahydro-2-furanylcarbonyl]octahydro-2H-pyrazino[1,2-a]pyrazine-2-carboxylate

NMR (CDCl₃, δ): 1.10-1.60 (9H, m), 1.80-2.15 (4H, m), 2.15-3.35 (8H, m), 3.35-4.30 (7H, m), 4.40-4.60 (1H, m), 7.10-7.40 (10H, m)

MASS (API-ES, Pos): 506.4 (M+H)⁺, 528.3 (M+Na)⁺

### (9) tert-Butyl (4R, 9aS)-4-benzhydryl-8-(tetrahydro-3-furanylcarbonyl)octahydro-2H-pyrazino[1,2-a]pyrazine-2-carboxylate

NMR (CDCl₃, δ): 1.10-1.60 (9H, m), 1.85-3.30 (11H, m), 3.45-4.40 (9H, m), 7.10-7.40 (10H, m)

MASS (API-ES, Pos): 506.2 (M+H)⁺, 528.3 (M+Na)⁺(free)

### (10) tert-Butyl (4R, 9aS)-4-benzhydryl-8-[(3-methyl-3-oxetanyl)carbonyl]octahydro-2H-pyrazino[1,2-a]pyrazine-2-carboxylate

NMR (CDCl₃, δ): 1.10-1.50 (9H, m), 1.50-1.70 (3H, m), 2.00 (1H, br s), 2.25-3.25 (8H, m), 3.40-3.90 (2H, m), 4.00-4.40 (4H, m), 4.80-5.00 (2H, m), 7.10-7.40 (10H, m)

MASS (API-ES, Pos): 506.4 (M+H)⁺, 528.3 (M+Na)⁺

### (11) tert-Butyl (4R, 9aS)-4-benzhydryl-8-[(3-ethyl-3-oxetanyl)carbonyl]octahydro-2H-pyrazino[1,2-a]pyrazine-2-carboxylate

NMR (CDCl₃, δ): 1.85-1.05 (3H, m), 1.05-1.70 (9H, m), 1.80-2.15 (3H, m), 2.25-2.55 (1H, m), 2.55-3.20 (7H, m), 3.40-3.90 (2H, m), 4.05-4.40 (4H, m), 4.80-5.00 (2H, m), 7.10-7.40 (10H, m)

MASS (API-ES, Pos): 520.3 (M+H)⁺, 542.3 (M+Na)⁺

### (12) tert-Butyl (4R, 9aS)-4-benzhydryl-8-(trifluoroacetyl)-octahydro-2H-pyrazino[1,2-a]pyrazine-2-carboxylate

### (13) tert-Butyl (4R, 9aS)-4-benzhydryl-8-(1,3-dioxan-5-ylcarbonyl)octahydro-2H-pyrazino[1,2-a]pyrazine-2-carboxylate

NMR (CDCl₃, δ): 1.15-1.60 (9H, m), 1.80-2.10 (1H, m), 2.25-3.30 (8H, m), 3.45-4.35 (9H, m), 4.60-4.75 (1H, m), 4.95-5.10 (1H, m), 7.10-7.40 (10H, m)

MASS (API-ES, Pos): 522.2 (M+H)⁺, 544.3 (M+Na)⁺

Preparation 112

To an ice-cooled solution of tert-butyl (4R, 9aS)-4-benzhydryl-8-(2-pyrazinylcarbonyl)octahydro-2H-pyrazino-[1,2-a]pyrazine-2-carboxylate (780 mg) in dichloromethane (10 ml) was added dropwise 4N hydrogen chloride in ethyl acetate (3.43 ml). After being stirred at room temperature for 1 hour, the reaction mixture was concentrated under reduced pressure. The residue was partitioned between dichloromethane and aqueous sodium hydrogen carbonate. The organic layer was dried over sodium sulfate, and concentrated in vacuo to give (6R, 9aR)-6-benzhydryl-2-(2-pyrazinylcarbonyl)octahydro-2H-pyrazino[1,2-a]pyrazine as a colorless foam (535 mg).

NMR (CDCl₃, δ): 1.90-4.50 (14H, m), 7.10-7.40 (10H, m), 8.45-8.55 (1H, m), 8.57-8.65 (1H, m), 8.85-8.95 (1H, m)

MASS (API-ES): 414 (M+H)⁺

Preparation 113

The following compounds were obtained according to a similar manner to that of Preparation 112.

### (1) (6R, 9aR)-6-Benzhydryl-2-[(4-oxido-2-pyrazinyl)carbonyl]octahydro-2H-pyrazino[1,2-a]pyrazine

NMR (CDCl₃, δ): 1.90-2.10 (1H, m), 2.47-2.70 (5H, m), 2.82-3.20 (5H, m), 3.65-3.83 (1H, m), 4.17-4.26 (2H, m), 7.16-7.27 (10H, m), 8.04-8.09 (1H, m), 8.33-8.39 (2H, m)

MASS (ES positive): 430 (M+H)⁺

### (2) N-[2-(−6R, 9aR)-6-Benzhydryloctahydro-2H-pyrazino[1,2-a]-pyrazin-2-yl]-1-(hydroxymethyl)-2-oxoethyl]acetamide

NMR (CDCl₃, δ): 1.93-2.17 (5H, m), 2.17-3.32 (10H, m), 3.56-3.90 (3H, m), 4.00-4.31 (2H, m), 4.76-4.93 (1H, m), 6.70-6.83 (1H, m), 7.15-7.26 (10H, m)

MASS (ES positive): 437 (M+H)⁺

### (3) N-[1-[[(6R, 9aR)-6-Benzhydryloctahydro-2H-pyrazino-[1,2-a]pyrazin-2-yl]carbonyl]-2-hydroxypropyl]acetamide

NMR (CDCl₃, δ): 1.09-1.13 (3H, m), 1.84-2.03 (1H, m), 2.03-2.05 (3H, m), 2.08-3.09 (11H, m), 3.65-4.24 (4H, m), 4.68-4.82 (1H, m), 6.31-6.39 (1H, m), 7.13-7.37 (10H, m)

MASS (ES positive): 451 (M+H)⁺

### (4) (6R, 9aS)-6-Benzhydryl-2-[(6-methoxy-2-pyrazinyl)carbonyl]octahydro-2H-pyrazino[1,2-a]pyrazine

NMR (CDCl₃, δ): 2.58-2.72 (5H, m), 2.82-3.27 (5H, m), 3.61-3.83 (2H, m), 3.83 and 3.95 (total 3H, each s), 4.10-4.36 (2H, m), 7.19-7.28 (10H, m), 8.23 and 8.27 (total 1H, each s), 8.39 and 8.41 (total 1H, each s)

MASS (ES positive): 444 (M+H)⁺

### (5) (6R, 9aR)-6-Benzhydryl-2-(1,4-dioxan-2-ylcarbonyl)octahydro-2H-pyrazino[1,2-a]pyrazine

NMR (CDCl₃, δ): 1.63 (9H, s), 1.80-2.10 (1H, m), 2.22-3.25 (9H, m), 3.45-3.95 (8H, m), 3.95-4.30 (3H, m), 7.15-7.27 (10H, m)

MASS (ES positive): 422 (M+H)⁺

### (6) (6R, 9aR)-6-Benzhydryl-2-[(2R)-tetrahydro-2-furanylcarbonyl]octahydro-2H-pyrazino[1,2-a]pyrazine

NMR (CDCl₃, δ): 1.80-2.40 (6H, m), 2.45-3.15 (8H, m), 3.60-4.00 (3H, m), 4.05-4.30 (2H, m), 4.45-4.65 (1H, m), 7.10-7.40 (10H, m)

MASS (API-ES, Pos): 406.3 (M+H)⁺, 428.2 (M+Na)⁺

### (7) 3-[[(6R, 9aR)-6-Benzhydryloctahydro-2H-pyrazino[1,2-a]-pyrazin-2-yl]carbonyl]-2-pyrazinamine

NMR (CDCl₃, δ): 1.90-2.10 (1H, m), 2.35-4.05 (10H, m), 4.15-4.40 (2H, m), 5.70 (2H, s), 7.05-7.40 (10H, m), 7.70-7.90 (1H, m), 7.95-8.10 (1H, m)

MASS (API-ES, Pos): 429.2 (M+H)⁺, 451.3 (M+Na)⁺

### (8) (6R, 9aR)-6-Benzhydryl-2-[(2S)-tetrahydro-2-furanylcarbonyl]octahydro-2H-pyrazino[1,2-a]pyrazine

NMR (CDCl₃, δ): 1.60-2.10 (3H, m), 2.10-2.50 (2H, m), 2.50-3.30 (8H, m), 3.50-4.00 (4H, m), 4.00-4.30 (2H, m), 4.45-4.65 (1H, m), 7.10-7.40 (10H, m)

MASS (API-ES, Pos): 406.3 (M+H)⁺, 428.2 (M+Na)⁺

### (9) (6R, 9aR)-6-Benzhydryl-2-tetrahydro-3-furanylcarbonyl)octahydro-2H-pyrazino[1,2-a]pyrazine

NMR (CDCl₃, δ): 1.80-2.35 (4H, m), 2.45-3.25 (9H, m), 3.40-4.05 (5H, m), 4.10-4.30 (2H, m), 7.10-7.40 (10H, m)

MASS (API-ES, Pos): 406.3 (M+H)⁺, 428.2 (M+Na)⁺

### (10) (6R, 9aR)-6-Benzhydryl-2-[(3-methyl-3-oxetanyl)carbonyl]octahydro-2H-pyrazino[1,2-a]pyrazine

NMR (CDCl₃, δ): 1.40-2.10 (4H, m), 2.20-3.20 (10H, m), 4.00-4.40 (4H, m), 4.80-5.05 (2H, m), 7.05-7.45 (10H, m)

MASS (API-ES, Pos): 406.3 (M+H)⁺, 428.2 (M+Na)⁺

### (11) (6R, 9aR)-6-Benzhydryl-2-[(3-ethyl-3-oxetanyl)-carbonyl]octahydro-2H-pyrazino[1,2-a]pyrazine

NMR (CDCl₃, δ): 0.94 (3H, t, J=7.4 Hz), 1.80-2.10 (3H, m), 2.15-2.40 (1H, m), 2.45-3.15 (9H, m), 4.05-4.40 (4H, m), 4.80-5.00 (2H, m), 7.10-7.40 (10H, m)

MASS (API-ES, Pos): 420.2 (M+H)⁺, 442.3 (M+Na)⁺

### (12) (6R, 9aR)-6-Benzhydryl-2-(trifluoroacetyl)octahydro-2H-pyrazino[1,2-a]pyrazine

NMR (CDCl₃, δ): 1.95-2.10 (1H, m), 2.35-2.50 (1H, m), 2.50-2.75 (3H, m), 2.75-3.30 (5H, m), 3.55-3.80 (1H, m), 4.00-4.30 (2H, m), 7.10-7.40 (10H, m)

MASS (API-ES, Pos): 404.2 (M+H)⁺, 426.2 (M+Na)⁺

### (13) (6R, 9aR)-6-Benzhydryl-N,N-dimethyloctahydro-2H-pyrazino[1,2-a]pyrazine-2-carboxamide

NMR (CDCl₃, δ): 1.85-2.06 (1H, m), 2.28-3.45 (11H, m), 2.78 (6H, s), 4.24 (1H, d, J=6.9 Hz), 7.08-7.36 (10H, m)

MASS (ES+): 379 (M+H)⁺

### (14) (6R, 9aR)-6-Benzhydryl-2-(0,1,3-dioxan-5-ylcarbonyl)-octahydro-2H-pyrazino[1,2-a]pyrazine

NMR (CDCl₃, δ): 1.80-2.00 (1H, m), 2.20-2.40 (1H, m), 2.45-3.25 (9H, m), 3.55-3.70 (1H, m), 3.75-4.30 (6H, m), 4.60-4.70 (1H, m), 4.95-5.05 (1H, m), 7.10-7.35 (10H, m)

MASS (API-ES, Pos): 422.2 (M+H)⁺, 444.4 (M+Na)⁺

Preparation 114

To an ice-cooling mixture of tert-butyl (4R, 9aS)-4-benzhydryloctahydro-2H-pyrazino[1,2-a]pyrazine-2-carboxylate (350 mg) and N,N-dimethylglycine (73.5 mg) in dichloromethane (10 ml) were added 1-hydroxybenzotriazole (139 mg) and 1-(3-dimethylaminopropyl)-3-ethylcarbodiimide hydrochloride (198 mg), and the whole was stirred overnight. The reaction mixture was washed with aqueous sodium hydrogen carbonate, and brine successively. The organic layer was separated, dried over magnesium sulfate and evaporated under reduced pressure. The residue was purified by column chromatography on silica gel using a mixed solvent of dichloromethane and methanol (40:1). The fractions containing the objective compound were collected and concentrated under reduced pressure. The residue was treated with 4N hydrogen chloride in dioxane (3 ml), the resulting precipitate was collected by filtration, washed with diisopropyl ether, and dried in vacuo to give a powder of 2-[(6R, 9aR)-6-benzhydryloctahydro-2H-pyrazino[1,2-a]-pyrazin-2-yl]-N,N-dimethyl-2-oxoethanamine trihydrochloride (38 mg).

NMR (CDCl₃, δ): 1.20-1.45 (9H, br s), 1.80-4.20 (21H, m), 7.18-7.30 (10H, m)

MASS (API-ES, Pos): 493 (M+H)

Preparation 115

To a solution of 4-methoxy-6-(2,2,2-trifluoroethoxy)-2-pyrimidinamine (2.1 g) in acetic acid (14 ml) was added portionwise sodium nitrile (1.3 g) with maintaining reaction temperature below 30° C., and then the mixture was stirred at room temperature for 15 hours. After the reaction mixture was concentrated under reduced pressure, dichloromethane and water were added to the residue with stirring, the pH of the aqueous layer was adjusted to 3 with saturated aqueous sodium hydrogen carbonate. The organic layer was separated, dried over sodium sulfate, and concentrated under reduced pressure to give crude solid. The solid was dissolved into minimum ethyl acetate, and triturated with a mixed solvent of diisopropyl ether and ethyl acetate (20:1) to give yellowish powder of 4-methoxy-6-(2,2,2-trifluoroethoxy)-2-pyrimidinol (1.31 g).

NMR (DMSO-d₆, δ): 3.86 (3H, s), 4.95 (2H, q, J=9.1 Hz), 5.74 (1H, s), 11.98 (1H, br)

MASS (API-ES): 247 (M+Na)⁺

Preparation 116

A mixture of 4-methoxy-6-(2,2,2-trifluoroethoxy)-2-pyrimidinol (700 mg), and ethyl iodide (1.46 g) and cesium fluoride (1.42 g) in N,N-dimethylformamide (15 ml) was stirred at 43° C. for 4 hours. After being cooled to room temperature, the mixture was poured into water (70 ml), and extracted with ethyl acetate (70 ml). The extract was dried over sodium sulfate and evaporated under reduced pressure. The resulting residue was purified by column chromatography on silica gel (15 g) using a mixed solvent of hexane and ethyl acetate (4:1). The fractions containing the objective compound were collected and evaporated under reduced pressure to give 2-ethoxy-4-methoxy-6-(2,2,2-trifluoroethoxy)pyrimidine as an oil (680 mg).

NMR (CDCl₃, δ): 1.43 (3H, t, J=7.1 Hz), 3.94 (3H, s), 4.40 (2H, q, J=7.1 Hz), 4.75 (2H, q, J=8.5 Hz), 5.83 (1H, s)

MASS (API-ES): 275 (M+Na)⁺

Preparation 117

The following compounds were obtained according to a similar manner to that of Preparation 116.

### (1) 4-Isopropoxy-6-methoxy-2-(2,2,2-trifluoroethoxy)-5-pyrimidinecarbaldehyde

### (2) 2-(2,2-Difluoroethoxy)-4-isopropoxy-6-methoy-5-pyrimidinecarbaldehyde

NMR (CDCl₃, δ): 1.42 (6H, d, J=6.1 Hz), 4.08 (3H, s), 4.59 (2H, dt, J=4.1, 13 Hz), 5.35-5.60 (1H, m), 6.14 (1H, tt, J=4.1, 55 Hz), 10.21 (1H, s)

MASS (API-ES): 299 (M+Na)⁺

### (3) 2-(2,2-Difluoroethoxy)-4-ethoxy-6-methoxy-5-pyrimidinecarbaldehyde

NMR (CDCl₃, δ): 1.45 (3H, t, J=7.1 Hz), 4.09 (3H, s), 4.55 (2H, q, J=7.1 Hz), 4.60 (2H, dt, J=4.1, 13 Hz), 6.14 (1H, tt, J=4.1, 55 Hz), 10.23 (1H, s)

MASS (API-ES): 285 (M+Na)⁺

### (4) 2-Cyclopropyl-4-ethoxy-6-methoxypyrimidine

NMR (CDCl₃, δ): 0.92-1.00 (2H, m), 1.07-1.14 (2H, m), 1.36 (3H, t, J=7.1 Hz), 2.00-2.08 (1H, m), 3.89 (3H, s), 4.31 (2H, q, J=7.1 Hz), 5.77 (1H, s)

### (5) 2-Cyclopropyl-4-isopropoxy-6-methoxypyrimidine

NMR (CDCl₃, δ): 0.90-0.99 (2H, m), 1.05-1.13 (2H, m), 1.31 (6H, d, J=6.3 Hz), 2.00-2.08 (1H, m), 3.88 (3H, s), 5.23 (1H, sept, J=6.3 Hz), 5.74 (1H, s)

Preparation 118

To a solution of 2-ethoxy-4-hydroxy-6-methoxy-5-pyrimidinecarbaldehyde (500 mg) in N,N-dimethylformamide (10 ml) were added 2-bromo-1,1-difluoroethane (1.83 g), cesium fluoride (1.15 g) and potassium iodide (419 mg) at room temperature. The mixed solution was stirred at 43° C. for 6 hours. 2-Bromo-1,1-difluoroethane (1.0 g) was added again to the solution at room temperature, and then the whole was stirred at 61° C. for 2 days. After being cooled to room temperature, the mixture was poured into water (50 ml). The resulting mixture was extracted with ethyl acetate (50 ml). The extract was washed with brine, dried over sodium sulfate and evaporated under reduced pressure. The residue was purified by column chromatography on silica gel (20 g) using a mixed solvent of hexane and ethyl acetate (1:1). The fractions containing the objective compound were collected and evaporated under reduced pressure to give crude solid. The solid was dissolved into minimum ethyl acetate, and triturated with diisopropyl ether to give colorless solid of 4-(2,2-difluoroethoxy)-2-ethoxy-6-methoxy-5-pyrimidinecarbaldehyde (55 mg).

NMR (CDCl₃, δ): 1.45 (3H, t, J=7.1 Hz), 4.09 (3H, s), 4.46 (2H, q, J=7.1 Hz), 4.67 (2H, dt, J=4.1, 13 Hz), 6.15 (1H, tt, J=4.1, 55 Hz), 10.22 (1H, s)

MASS (API-ES): 285 (M+Na)⁺

Preparation 119

The following compounds were obtained according to a similar manner to that of Preparation 118.

### (1) 2-(2,2-Difluoroethoxy)-4,6-dimethoxypyrimidine

NMR (CDCl₃, δ): 3.94 (6H, s), 4.55 (2H, dt, J=4.3, 13 Hz), 5.77 (1H, s), 6.15 (1H, tt, J=4.3, 55 Hz)

MASS (API-ES): 243 (M+Na)⁺

### (2) 2-Cyclopropyl-4-(2,2-difluoroethoxy)-6-methoxypyrimidine

NMR (CDCl₃, δ): 0.97-1.01 (2H, m), 1.07-1.11 (2H, m), 2.02-2.07 (1H, m), 3.90 (3H, s), 4.52 (2H, dt, J=13.5, 4.24 HZ), 5.87 (1H, s), 6.07 (1H, tt, J=55.5, 4.24 Hz)

### (3) 2-Cyclopropyl-4-(2-fluoroethoxy)-6-methoxypyrimidine

IR (Neat): 1577, 1568, 1466, 1425, 1390, 1350, 1257, 1188, 1169, 1051 cm⁻¹

NMR (CDCl₃, δ): 0.90-1.15 (4H, m), 1.90-2.14 (1H, m), 3.90 (3H, s), 4.42-4.86 (4H, m), 5.86 (1H, s)

MASS (ES+): 235 (M+Na)⁺, 213 (M+H)⁺

### (4) 4-(2-Fluoroethoxy)-2,6-dimethoxypyrimidine

IR (Neat): 1595, 1587, 1369, 1346, 1169, 1105 cm⁻¹

NMR (CDCl₃, δ): 3.93 (3H, s), 3.96 (3H, s), 4.45-4.90 (4H, m), 5.78 (1H, s)

MASS (ES+): 225 (M+Na)⁺, 203 (M+H)⁺

### (5) 2-(2-Fluoroethoxy)-4-methoxy-6-(2,2,2-trifluoroethoxy)pyrimidine

IR (Neat): 1604, 1577, 1342, 1163, 1115, 1055 cm⁻¹

NMR (CDCl₃, δ): 3.95 (3H, s), 4.46-4.94 (6H, m), 5.87 (1H, s)

MASS (ES+): 293 (M+Na)⁺, 271 (M+H)⁺

Preparation 120

To an ice-cooled solution of 2,2,2-trifluoroethanol (292 mg) in tetrahydrofuran (5 ml) was added portionwise 60% sodium hydride in oil (129 mg). The mixture was stirred below 5° C. for 30 minutes, then at room temperature for 10 minutes. To the ice-cooled solution was added a solution of 2-chloro-4,6-dimethoxypyrimidine (510 mg) in tetrahydrofuran (3 ml), and the mixture was stirred below 5° C. for 10 minutes, at room temperature for 3 hours. The mixture was poured into a mixture of water (15 ml) and ethyl acetate (10 ml). The organic layer was separated and dried over magnesium sulfate, filtered and evaporated under reduced pressure to give crude oil. The oil was purified by column chromatography (silica gel 20 g, ethyl acetate/hexane (1/6)) to give 4,6-dimethoxy-2-(2,2,2-trifluoroethoxy)pyrimidine as powder (420 mg).

NMR (CDCl₃, δ): 3.94 (6H, s), 4.77 (2H, q, J=8.4 Hz), 5.79 (1H, s)

MASS (API-ES): 261 (M+Na)⁺

Preparation 121

The following compounds were obtained according to a similar manner to that of Preparation 120.

### (1) 2,14-Dimethoxy-6-(2,2,2-trifluoroethoxy)pyrimidine

NMR (CDCl₃, δ): 3.95 (3H, s), 4.00 (3H, s), 4.76 (2H, q, J=8.4 Hz), 5.85 (1H, s)

MASS (API-ES): 239 (M+H)⁺

### (2) 4-Methoxy-6-(2,2,2-trifluoroethoxy)-2-pyrimidinamine

NMR (CDCl₃, δ): 3.86 (3H, s), 4.68 (2H, q, J=8.5 Hz), 4.89 (2H, br), 5.58 (1H, s)

MASS (API-ES): 224 (M+H)⁺

Preparation 122

To an ice-cooled solution of 4,6-dimethoxy-2-(2,2,2-trifluoroethoxy)pyrimidine (410 mg) in N,N-dimethylformamide (5 ml) was added dropwise phosphorus oxychloride (660 mg) under nitrogen atmosphere. The mixed solution was stirred at room temperature for 2 days, then at 46° C. for 3 hours. Phosphorus oxychloride (200 mg) was added again to the solution, and it was stirred at 52° C. for 6 hours. The reaction mixture was quenched with ice-water (20 ml), and the whole was extracted with ethyl acetate (20 ml), the extract was washed with brine, dried over sodium sulfate, and concentrated under reduced pressure. The resulting residue was purified by column chromatography on silica gel (11 g) using a mixed solvent of hexane and ethyl acetate (4:1 to 1:1). The fractions containing the objective compound were collected and concentrated under reduced pressure to give yellowish solid of 4,6-dimethoxy-2-(2,2,2-trifluoroethoxy)-5-pyrimidinecarbaldehyde (190 mg).

NMR (CDCl₃, δ): 4.10 (6H, s), 4.83 (2H, q, J=8.2 Hz), 10.23 (1H, s)

MASS (API-ES): 289 (M+Na)⁺

Preparation 123

The following compounds were obtained according to a similar manner to that of Preparation 122.

### (1) 2,4-Dimethoxy-6-(2,2,2-trifluoroethoxy)-5-pyrimidinecarbaldehyde

NMR (CDCl₃, δ): 4.06 (3H, s), 4.12 (3H, s), 4.89 (2H, q, J=8.3 Hz), 10.23 (1H, s)

MASS (API-ES): 289 (M+Na)⁺

### (2) 2-Ethoxy-4-methoxy-6-(2,2,2-trifluoroethoxy)-5-pyrimidinecarbaldehyde

NMR (CDCl₃, δ): 1.46 (3H, t, J=7.1 Hz), 4.12 (3H, s), 4.49 (2H, q, J=7.1 Hz), 4.88 (2H, q, J=8.3 Hz), 10.23 (1H, s)

MASS (API-ES): 303 (M+Na)⁺

### (3) 2-(2,2-Difluoroethoxy)-4,6-dimethoxy-5-pyrimidinecarbaldehyde

NMR (CDCl₃, δ): 4.10 (6H, s), 4.62 (2H, dt, J=4.3, 13 Hz), 6.15 (1H, tt, J=4.3, 55 Hz), 10.22 (1H, s)

MASS (API-ES): 271 (M+Na)⁺

### (4) 2-Cyclopropyl-4-ethoxy-6-methoxypyrimidinecarbaldehyde

NMR (CDCl₃, δ): 1.07-1.11 (2H, m), 1.17-1.21 (2H, m), 1.41 (3H, t, J=7.1 Hz), 2.07-2.11 (1H, m), 4.03 (3H, s), 4.51 (2H, q, J=7.1 Hz), 10.28 (1H, s)

### (5) 2-Cyclopropyl-4-isopropoxy-6-methoxy-5-pyrimidinecarbaldehyde

NMR (CDCl₃, δ): 1.04-1.11 (2H, m), 1.16-1.23 (2H, m), 1.38 (6H, d, J=6.3 Hz), 2.03-2.11 (1H, m), 4.03 (3H, s), 5.47 (1H, sept, J=6.3 Hz), 10.26 (1H, s)

Preparation 124

Under nitrogen atmosphere, to an ice-cooled solution of 4,6-dimethoxy-2-(2,2,2-trifluoroethoxy)-5-pyrimidinecarbaldehyde (178 mg) in dichloromethane (3 ml) was added 1.0M boron tribromide in dichloromethane (0.84 ml). After it was stirred at room temperature for 10 minutes, 1.0M boron tribromide in dichloromethane (0.67 ml) was added again to the solution at the same temperature, and then the whole was stirred for 30 minutes. The reaction mixture was poured into ice-water, the pH of the aqueous layer was adjusted to 3 with aqueous sodium hydrogen carbonate. The organic layer was separated, dried over sodium sulfate, and concentrated under reduced pressure. The resulting residue was purified by column chromatography on silica gel (6 g) using a mixed solvent of dichloromethane and methanol (10:1). The fractions containing the objective compound were collected and concentrated under reduced pressure to give 4-hydroxy-6-methoxy-2-(2,2,2-trifluoroethoxy)-5-pyrimidinecarbaldehyde as slight red solid (150 mg).

NMR (CDCl₃, δ): 4.13 (3H, s), 4.84 (2H, q, J=8.1 Hz), 10.05 (1H, s), 13.10 (1H, br)

MASS (API-ES): 275 (M+Na)⁺

Preparation 125

The following compound was obtained according to a similar manner to that of Preparation 124.

### 2-(2,2-Difluoroethoxy)-4-hydroxy-6-methoxy-5-pyrimidinecarbaldehyde

NMR (CDCl₃, δ): 4.12 (3H, s), 4.63 (2H, dt, J=4.1, 13 Hz), 6.13 (1H, tt, J=4.1, 55 Hz), 10.04 (1H, s), 13.15 (1H, br)

MASS (API-ES): 257 (M+Na)⁺

Preparation 126

A mixture of 2-cyclopropyl-6-methoxy-4-pyrimidinol (420 mg), 2,2,2-trifluoroethyl-p-toluenesulfonate (1.76 g) and cesium fluoride (1.15 g) in N,N-dimethylformamide (8 ml) was stirred at 60° C. for 3 hours. After being cooled to room temperature, the mixture was poured into water, and extracted with ethyl acetate. The extract was dried over magnesium sulfate and evaporated under reduced pressure. The resulting residue was purified by column chromatography on silica gel (15 g) using a mixed solvent of hexane and ethyl acetate (4:1). The fractions containing the objective compound were collected and evaporated under reduced pressure to give an oil of 2-cyclopropyl-4-methoxy-6-(2,2,2-trifluoroethoxy)pyrimidine (435 mg).

NMR (CDCl₃, δ): 0.98-1.12 (4H, m), 2.00-2.10 (1H, m), 3.91 (3H, s), 4.75 (1H, AB q, J=8.5 Hz), 5.92 (1H, s)

MASS (API-ES, Pos): 249 (M+H)

Preparation 127

A mixture of 2-cyclopropyl-4-methoxy-6-(2,2,2-trifluoethoxy)pyrimidine (250 mg) and N-bromosuccinimide (359 mg) in acetic acid (0.577 ml) was stirred at 60° C. for 3.5 hours. After being cooled to room temperature, the mixture was poured into water, and extracted with ethyl acetate. The extract was dried over magnesium sulfate and evaporated under reduced pressure. The resulting residue was purified by column chromatography on silica gel using a mixed solvent of hexane and ethyl acetate (4:1). The fractions containing the objective compound were collected and evaporated under reduced pressure to give an oil of 5-bromo-2-cyclopropyl-4-methoxy-6-(2,2,2-trifluoroethoxy)-pyrimidine (250 mg).

NMR (CDCl₃, δ): 0.98-1.13 (4H, m), 2.00-2.11 (1H, m), 4.01 (3H, s), 4.80 (1H, AB q, J=8.6 Hz)

MASS (API-ES, Pos): 329 (M+Na)⁺

Preparation 128

The following compounds were obtained according to a similar manner to that of Preparation 127.

### (1) 5-Bromo-2-cyclopropyl-4-(2,2-difluoroethoxy)-6-methoxypyrimidine

NMR (CDCl₃, δ): 0.97-1.09 (4H, m), 1.98-2.11 (1H, m), 4.00 (3H, s), 4.58 (2H, dt, J=13.1, 4.12 Hz), 6.11 (1H, tt, J=55.3, 4.18 Hz)

### (2) 5-Bromo-2-cyclopropyl-4-(2-fluoroethoxy)-6-methoxypyrimidine

NMR (CDCl₃, δ): 0.95-1.11 (4H, m), 1.96-2.09 (1H, m), 3.99 (3H, s), 4.55-4.64 (2H, m), 4.68-4.72 (1H, m), 4.84-4.88 (1H, m)

MASS (ES positive): 291 (M+H)⁺

Preparation 129

To a dry ice-acetone cooling solution of 5-bromo-2-cyclopropyl-4-methoxy-6-(2,2,2-trifluoroethoxy)pyrimidine (290 mg) in tetrahydrofuran was added a solution of 1.5M butyl lithium in hexane (0.89 ml) dropwise below −65° C. After being stirred below −70° C. for 0.5 hour, N,N-dimethylformamide (0.686 ml) was added to the mixture, and the whole was stirred at −70° C., followed by warmed to −5° C. over 10 minutes. To the mixture was added diluted hydrochloric acid, and the whole was extracted with ethyl acetate. The extract was dried over magnesium sulfate and evaporated under reduced pressure. The resulting residue was purified by column chromatography on silica gel using a mixed solvent of toluene and ethyl acetate (50:1). The fractions containing the objective compound were collected and evaporated under reduced pressure to give an oil of 2-cyclopropyl-4-methoxy-6-(2,2,2-trifluoroethoxy)-5-pyrimidinecarbaldehyde (50 mg).

NMR (CDCl₃, δ): 1.08-1.26 (4H, m), 2.06-2.16 (1H, m), 4.06 (3H, s), 4.87 (1H, AB q, J=8.3 Hz), 10.29 (1H, s)

MASS (API-ES, Pos): 299 (M+Na)

Preparation 130

The following compounds were obtained according to a similar manner to that of Preparation 129.

### (1) 2-Cyclopropyl-4-(2,2-difluoroethoxy)-6-methoxy-5-pyrimidinecarbaldehyde

NMR (CDCl₃, δ): 1.07-1.25 (4H, m), 2.05-2.15 (1H, m), 4.06 (3H, s), 4.65 (1H, dt, J=13.1, 4.1 Hz), 6.14 (1H, tt, J=55.2, 4.08 Hz), 10.28 (1H, s)

### (2) 2-Cyclopropyl-4-(2-fluoroethoxy)-6-methoxy-5-pyrimidinecarbaldehyde

NMR (CDCl₃, δ): 1.05-1.24 (4H, m), 2.03-2.13 (1H, m), 4.05 (3H, s), 4.64 (2H, s), 4.75-4.80 (1H, m), 4.85-4.89 (1H, m), 10.30 (1H, m)

MASS (ES positive): 263 (M+Na)⁺

### (3) 4-(2-Fluoroethoxy)-2,6-dimethoxy-5-pyrimidinecarbaldehyde

mp: 127-129° C.

IR (KBr): 1691, 1682, 1595, 1562, 1155 cm⁻¹

NMR (CDCl₃, δ): 4.04 (3H, s), 4.10 (3H, s), 4.56-4.95 (4H, m), 10.25 (1H, s)

MASS (ES+): 231 (M+H)⁺

### (4) 2-(2-Fluoroethoxy)-4-methoxy-6-(2,2,2-trifluoroethoxy)-5-pyrimidinecarbaldehyde

mp: 63-65° C.

IR (KBr): 1695, 1587, 1562, 1171, 1136 cm⁻¹

NMR (CDCl₃, δ): 4.11 (3H, s), 4.65-4.98 (6H, m), 10.23 (1H, s)

MASS (ES+): 321 (M+Na)⁺, 299 (M+H)⁺

Preparation 131

A 1.6M solution of butyl lithium in hexane (3.51 ml) was added to cold (−10° C.) tetrahydrofuran (40 ml). To the solution was added 2,2,6,6-tetramethylpiperidine (832 mg) at −70° C. The mixture was warmed to 0° C. and kept at 0° C. for 30 minutes. It was then cooled to −70° C. and to it was added 2-ethoxypyrazine (484 mg), and the mixture was stirred for 30 minutes at −70° C. A stream of carbon dioxide (supplied by evaporation of solid carbon dioxide) was passed through the solution for 30 minutes. Hydrolysis was then carried out at −70° C. using a mixture of 35% aqueous hydrochloric acid (2 ml), ethanol (2 ml), and tetrahydrofuran (8 ml). The mixture was then gently warmed to room temperature, neutralized with saturated aqueous sodium hydrogen carbonate (10 ml), and evaporated nearly to dryness. The residue was extracted with dichloromethane (50 ml×3). The combined organic extracts were dried over magnesium sulfate and evaporated. The residue was azeotropically evaporated with toluene (100 ml×2). The residue was dissolved in a mixture of ethyl acetate and methanol (60 ml-40 ml) and undissolved precipitates were removed off by filtration. The filtered solution was evaporated and the precipitates collected were washed with dichloromethane to give 3-methoxy-2-pyrazinecarboxylic acid (160 mg).

NMR (CDCl₃, δ): 8.27 (1H, d, J=2.57 Hz), 8.40 (1H, d, J=2.58 Hz)

MASS (ES positive): 153 (M−H)⁺

Preparation 132

A mixture of 3-(benzyloxy)-1,2-propanediol (2.56 g) and 1,2-bis(4-methylbenzenesulfonyloxy)ethane (5.2 g) in 2,6-di-tert-butylpyridine (10.8 g) was heated at 180° C. for 4 hours. After being cooled to room temperature, the mixture was partitioned between ethyl acetate and water. The organic phase was washed with saturated aqueous sodium hydrogen carbonate and brine, dried over magnesium sulfate, and evaporated in vacuo. The residue was purified by chromatography on silica gel eluting first with ethyl acetate-hexane (1:20) to remove di-tert-butylpyridine, and then with ethyl acetate-hexane (1:10) to give 2-[(benzyloxy)methyl]-1,4-dioxane (1.47 g).

NMR (CDCl₃, δ): 3.37-4.06 (7H, m), 4.54 (2H, s), 7.13-7.35 (5H, m)

MASS (ES positive): 231 (M+Na)⁺

Preparation 133

2-[(Benzyloxy)methyl]-1,4-dioxane (1.54 g) in methanol (15 ml) was hydrogenated over 10% palladium on carbon (50% wet, 200 mg) for 6 hours. The catalyst was removed off by filtration and the filtrate was evaporated in vacuo to give 1,4-dioxan-2-ylmethanol (736 mg).

NMR (CDCl₃, δ): 1.96 (1H, t, J=5.78 Hz), 3.46-3.81 (9H, m)

Preparation 134

To a solution of 1,4-dioxane-2-ylmethanol (650 mg) in a mixture of acetonitrile (6 ml) and water (6 ml) were added iodobenzene diacetate (3.9 g) and 2,2,6,6-tetramethyl-1-piperidinyloxy (172 mg) successively and the mixture was stirred at room temperature for 4 hours. The reaction mixture was diluted with ethyl acetate and the resulting solution was washed with water. The organic phase was extracted with 1N aqueous sodium hydroxide. The aqueous phases were combined and washed with ethyl acetate. The solution was brought to pH 3 with 1N aqueous hydrochloric acid, and it was extracted two times with ethyl acetate. The extracts were washed with brine, dried over magnesium sulfate, and evaporated in vacuo to give 1,4-dioxane-2-carboxylic acid (259 mg).

NMR (CDCl₃, δ): 3.65-3.78 (2H, m), 3.70-3.80 (2H, m), 3.96-4.03 (1H, m), 4.06-4.32 (1H, m), 4.31 (1H, dd, J=4.44, 1.64 Hz)

MASS (ES negative): 131 (M−H)⁺

Preparation 135

To a solution of methyl 6-methoxy-2-pyrazinecarboxylate (350 mg) was added 1N aqueous sodium hydroxide and the mixture was stirred at room temperature for 3 hours. The mixture was concentrated under reduced pressure. The residue was partitioned between ethyl acetate and 1N aqueous hydrochloric acid, and the organic phase was washed with brine, dried over magnesium sulfate, and evaporated in vacuo to give 6-methoxy-2-pyrazinecarboxylic acid (173 mg).

NMR (CDCl₃, δ): 3.96 (3H, s), 8.53 (1H, s), 8.76 (1H, s), 13.68 (1H, br s)

MASS (ES positive): 153 (M−H)+

Preparation 136

The following compound was obtained according to a similar manner to that of Preparation 51.

### Tert-Butyl (4R, 9aS)-4-benzhydryl-8-[(dimethylamino)-carbonyl]octahydro-2H-pyrazino[1,2-a]pyrazine-2-carboxylate

NMR (CDCl₃, δ): 1.61 (9H, s), 1.95-3.86 (12H, m), 2.78 (6H, s), 4.17 (1H, d, J=7.3 Hz), 7.10-7.35 (10H, m)

MASS (ES+): 479 (M+H)⁺

### EXAMPLE 7

To a solution of (6R, 9aR)-6-benzhydryl-2-(trifluoroacetyl)octahydro-2H-pyrazino[1,2-a]pyrazine in dichloromethane was added 2-ethoxy-4-methoxy-6-(2,2,2-trifluoroethoxy)-5-pyrimidinecarbaldehyde at 0° C., and stirred for 15 minutes at the same temperature, then added sodium triacetoxyborohydride and stirred for 3 hours at ambient temperature. The reaction mixture was added 2 ml of saturated sodium hydrogen carbonate aqueous solution, then extracted with dichloromethane (5 ml×3). The organic layer was dried over diatomaceous earth and evaporated under reduced pressure to give a crude oil. Purification by preparative TLC (0.5 mm silica gel, dichloromethane:methanol=10:04) gave (4R, 9aR)-4-benzhydryl-2-[[2-ethoxy-4-methoxy-6-(2,2,2-trifluoroethoxy)-5-pyrimidinyl]methyl]-8-(trifluoroacetyl)octahydro-2H-pyrazino[1,2-a]pyrazine as a colorless oil (117.9 mg).

NMR (CDCl₃, δ): 1.42 (3H, t, J=7.1 Hz), 1.85-2.05 (3H, m), 2.40-2.55 (2H, m), 2.60-3.00 (3H, m), 3.00-3.25 (1H, m), 3.25-3.35 (1H, m), 3.35-3.45 (2H, m), 3.55-3.70 (1H, m), 3.70-3.85 (3H, m), 3.90-4.20 (2H, m), 4.37 (2H, q, J=7.1 Hz), 4.50-4.80 (2H, m), 7.10-7.40 (10H, m)

MASS (API-ES, Pos): 668.2 (M+H)⁺, 690.1 (M+Na)⁺

### EXAMPLE 8

The following compounds were obtained according to a similar manner to that of Example 7.

### (1) (4R, 9aR)-4-Benzhydryl-2-[[2,4-dimethoxy-6-(2,2,2-trifluoroethoxy)-5-pyrimidinyl]methyl]-8-(trifluoroacetyl)octahydro-2H-pyrazino[1,2-a]pyrazine

NMR (CDCl₃, δ): 1.85-2.05 (3H, m), 2.40-3.80 (13H, m), 3.95 (3H, s), 4.05-4.15 (1H, m), 4.50-4.80 (2H, m), 7.10-7.35 (10H, m)

MASS (API-ES, Pos): 654.3 (M+H)⁺, 676.2 (M+Na)⁺

### (2) (4R, 9aR)-4-Benzhydryl-2-[(2-cyclopropyl-4-isopropoxy-6-methoxy-5-pyrimidinyl)methyl]-8-(trifluoroacetyl)-octahydro-2H-pyrazino[1,2-a]pyrazine

NMR (CDCl₃, δ): 0.85-1.00 (2H, m), 1.00-1.10 (2H, m), 1.15-1.25 (6H, m), 1.85-2.10 (4H, m), 2.40-4.15 (15H, m), 5.20-5.30 (1H, m), 7.10-7.30 (10H, m)

MASS (API-ES, Pos): 624.4 (M+H)⁺, 646.3 (M+Na)⁺

### (3) (4R, 9aR)-4-Benzhydryl-2-[[2-cyclopropyl-4-methoxy-6-(2,2,2-trifluoroethoxy)-5-pyrimidinyl]methyl]-8-[(2R)-tetrahydro-2-furanylcarbonyl]octahydro-2H-pyrazino-[1,2-a]pyrazine Dihydrochloride

NMR (DMSO-d₆, δ): 0.95-1.20 (4H, m), 1.65-4.70 (26H, m), 4.75-5.05 (2H, m), 7.05-7.55 (10H, m)

MASS (API-ES, Pos): 666.3 (M+H)⁺, 688.3 (M+Na)⁺(free)

### (4) (4R, 9aR)-4-Benzhydryl-2-[[2,4-dimethoxy-6-(2,2,2-trifluoroethoxy)-5-pyrimidinyl]methyl]-8-[(2R)-tetrahydro-2-furanylcarbonyl]octahydro-2H-pyrazino-[1,2-a]pyrazine Dihydrochloride

NMR (DMSO-d₆, δ): 1.70-1.90 (2H, m), 1.90-2.10 (2H, m), 2.10-4.75 (23H, m), 4.50-4.80 (1H, m), 4.80-5.05 (2H, m), 7.05-7.50 (10H, m)

MASS (API-ES, Pos): 656.2 (M+H)⁺, 678.3 (M+Na)⁺(free)

### (5) (4R, 9aR)-4-Benzhydryl-2-[(2-cyclopropyl-4-isopropoxy-6-methoxy-5-pyrimidinyl)methyl]-8-[(2R)-tetrahydro-2-furanylcarbonyl]octahydro-2H-pyrazino[1,2-a]pyrazine Dihydrochloride

NMR (DMSO-d₆, δ): 0.90-1.40 (10H, m), 1.70-4.50 (25H, m), 4.50-4.80 (1H, m), 5.05-5.20 (1H, m), 7.10-7.50 (10H, m)

MASS (API-ES, Pos): 626.3 (M+H)⁺, 648.4 (M+Na)⁺(free)

### (6) (4R, 9aR)-4-Benzhydryl-2-[(2-cyclopropyl-4-ethoxy-6-methoxy-5-pyrimidinyl)methyl]-8-[(2R)-tetrahydro-2-furanylcarbonyl]octahydro-2H-pyrazino[1,2-a]pyrazine Dihydrochloride

NMR (DMSO-d₆, δ): 0.90-1.30 (7H, m), 1.60-4.70 (28H, m), 7.00-7.50 (10H, m)

MASS (API-ES, Pos): 612.3 (M+H)⁺, 634.2 (M+Na)⁺(free)

### (7) (4R, 9aR)-4-Benzhydryl-2-[[2-cyclopropyl-4-(2,2-difluoroethoxy)-6-methoxy-5-pyrimidinyl]methyl]-8-[(2R)-tetrahydro-2-furanylcarbonyl]octahydro-2H-pyrazino[1,2-a]pyrazine Dihydrochloride

NMR (DMSO-d₆, δ): 0.90-1.15 (4H, m), 1.65-4.70 (28H, m), 6.10-6.55 (1H, m), 7.10-7.50 (10H, m)

MASS (API-ES, Pos): 648.3 (M+H)⁺, 670.3 (M+Na)⁺(free)

### (8) (4R, 9aR)-4-Benzhydryl-2-[[2-(2-fluoroethoxy)-4-methoxy-6-(2,2,2-trifluoroethoxy)-5-pyrimidinyl]methyl]-8-[(2R)-tetrahydro-2-furanylcarbonyl]octahydro-2H-pyrazino[1,2-a]pyrazine Dihydrochloride

NMR (DMSO-d₆, δ): 1.65-5.05 (31H, m), 7.10-7.50 (10H, m)

MASS (API-ES, Pos): 688.3 (M+H)⁺, 710.2 (M+Na)⁺(free)

### (9) (4R, 9aR)-4-Benzhydryl-2-[[2,4-dimethoxy-6-(2,2,2-trifluoroethoxy)-5-pyrimidinyl]methyl]-8-[(2S)-tetrahydro-2-furanylcarbonyl]octahydro-2H-pyrazino-[1,2-a]pyrazine Dihydrochloride

NMR (DMSO-d₆, δ): 1.70-4.40 (27H, m), 4.50-4.70 (1H, m), 4.80-5.10 (2H, m), 7.10-7.50 (10H, m)

MASS (API-ES, Pos): 656.2 (M+H)⁺, 678.2 (M+Na)⁺(free)

### (10) (4R, 9aR)-4-Benzhydryl-2-[(2-cyclopropyl-4-isopropoxy-6-methoxy-5-pyrimidinyl)methyl]-8-[(2S)-tetrahydro-2-furanylcarbonyl]octahydro-2H-pyrazino[1,2-a]pyrazine Dihydrochloride

NMR (DMSO-d₆, δ): 0.95-1.40 (10H, m), 1.70-4.70 (26H, m), 5.10-5.25 (1H, m), 7.10-7.50 (10H, m)

MASS (API-ES, Pos): 626.3 (M+H)⁺, 648.3 (M+Na)⁺(free)

### (11) (4R, 9aR)-4-Benzhydryl-2-[(2-cyclopropyl-4-ethoxy-6-methoxy-5-pyrimidinyl)methyl]-8-[(2S)-tetrahydro-2-furanylcarbonyl]octahydro-2H-pyrazino[1,2-a]pyrazine Dihydrochloride

NMR (DMSO-d₆, δ): 0.90-1.10 (4H, m), 1.10-1.30 (3H, m), 1.70-4.40 (27H, m), 4.50-4.65 (1H, m), 7.10-7.45 (10H, m)

MASS (API-ES, Pos): 612.4 (M+H)⁺, 634.4 (M+Na)⁺(free)

### (12) (4R, 9aR)-4-Benzhydryl-2-[[2,4-dimethoxy-6-(2,2,2-trifluoroethoxy)-5-pyrimidinyl]methyl]-8-(tetrahydro-3-furanylcarbonyl)octahydro-2H-pyrazino[1,2-a]pyrazine Dihydrochloride

NMR (DMSO-d₆, δ): 1.75-4.50 (28H, m), 4.80-5.05 (2H, m), 7.10-7.45 (10H, m)

MASS (API-ES, Pos): 656.2 (M+H)⁺, 678.1 (M+Na)⁺(free)

### (13) (4R, 9aR)-4-Benzhydryl-2-[(2-cyclopropyl-4-isopropoxy-6-methoxy-5-pyrimidinyl)methyl]-8-(tetrahydro-3-furanylcarbonyl)octahydro-2H-pyrazino[1,2-a]pyrazine Dihydrochloride

NMR (DMSO-d₆, δ): 0.90-1.40 (10H, m), 1.70-4.55 (26H, m), 5.05-5.25 (1H, m), 7.05-7.50 (10H, m)

MASS (API-ES, Pos): 626.2 (M+H)⁺, 648.4 (M+Na)⁺(free)

### (14) (4R, 9aR)-4-Benzhydryl-2-[(2-cyclopropyl-4-ethoxy-6-methoxy-5-pyrimidinyl)methyl]-8-(tetrahydro-3-furanylcarbonyl)octahydro-2H-pyrazino[1,2-a]pyrazine Dihydrochloride

NMR (DMSO-d₆, δ): 0.90-1.10 (4H, m), 1.10-1.30 (3H, m), 1.70-4.50 (28H, m), 7.10-7.50 (10H, m)

MASS (API-ES, Pos): 612.4 (M+H)⁺, 634.5 (M+Na)⁺(free)

### (15) (4R, 9aR)-4-Benzhydryl-2-[(2-ethoxy-4-isopropoxy-6-methoxy-5-pyrimidinyl)methyl]-8-[(3-ethyl-3-oxetanyl)carbonyl]octahydro-2H-pyrazino[1,2-a]pyrazine Dihydrochloride

NMR (DMSO-d₆, δ): 0.70-0.90 (3H, m), 1.05-1.30 (6H, m), 1.33 (3H, t, J=7.0 Hz), 1.70-4.50 (24H, m), 4.55-4.85 (2H, m), 5.05-5.25 (1H, m), 7.10-7.45 (10H, m)

MASS (API-ES, Pos): 644.3 (M+H)⁺, 666.2 (M+Na)⁺(free)

### (16) (6R, 9aR)-6-Benzhydryl-8-[[2,4-dimethoxy-6-(2,2,2-trifluoroethoxy)-5-pyrimidinyl]methyl]-N,N-dimethyloctahydro-2H-pyrazino[1,2-a]pyrazine-2-carboxamide Dihydrochloride

NMR (DMSO-d₆, δ): 2.00-4.55 (27H, m), 4.80-5.00 (2H, m), 7.10-7.40 (10H, m).

MASS (API-ES, Pos): 629.3 (M+H)⁺, 651.1 (M+Na)⁺(free)

### (17) (6R, 9aR)-6-Benzhydryl-8-[(2-cyclopropyl-4-isopropoxy-6-methoxy-5-pyrimidinyl)methyl]-N,N-dimethyloctahydro-2H-pyrazino[1,2-a]pyrazine-2-carboxamide Dihydrochloride

NMR (DMSO-d₆, δ): 0.85-1.10 (4H, m), 1.10-1.30 (6H, m), 1.95-5.00 (25H, m), 5.16 (1H, quintet, J=6.2 Hz), 7.10-7.55 (10H, m)

MASS (API-ES, Pos): 599.3 (M+H)⁺, 621.1 (M+Na)⁺(free)

### (18) 3-[[(6R, 9aR)-6-Benzhydryl-8-[[2-cyclopropyl-4-methoxy-6-(2,2,2-trifluoroethoxy)-5-pyrimidinyl]methyl]-octahydro-2H-pyrazino[1,2-a]pyrazin-2-yl]carbonyl]-2-pyrazinamine Trihydrochloride

NMR (DMSO-d₆, δ): 0.95-1.25 (4H, m), 1.90-4.60 (19H, m), 4.70-5.05 (2H, m), 7.15-7.50 (10H, m), 7.60-7.80 (1H, m), 8.00-8.15 (1H, m)

MASS (API-ES, Pos): 689.3 (M+H)⁺, 711.2 (M+Na)⁺(free)

### (19) (4R, 9aR)-4-Benzhydryl-2-[[2-cyclopropyl-4-methoxy-6-(2,2,2-trifluoroethoxy)-5-pyrimidinyl]methyl]-8-[(3-methyl-3-oxetanyl)carbonyl]octahydro-2H-pyrazino-[1,2-a]pyrazine Dihydrochloride

NMR (CDCl₃, δ): 0.95-1.20 (4H, m), 1.40-1.60 (3H, m), 1.90-4.45 (21H, m), 4.55-5.10 (4H, m), 7.10-7.50 (10H, m)

MASS (API-ES, Pos): 666.3 (M+H)⁺, 688.3 (M+Na)⁺

### (20) (4R, 9aR)-4-Benzhydryl-2-[(2-ethoxy-4-isopropoxy-6-methoxy-5-pyrimidinyl)methyl]-8-[(3-methyl-3-oxetanyl)carbonyl]octahydro-2H-pyrazino[1,2-a]pyrazine Dihydrochloride

NMR (CDCl₃, δ): 0.90-1.30 (6H, m), 1.32 (3H, t, J=7.0 Hz), 1.40-1.60 (3H, m), 2.10-2.35 (1H, m), 2.55-2.75 (2H, m), 2.75-3.40 (7H, m), 3.65-4.10 (6H, m), 4.10-4.30 (4H, m), 4.33 (2H, q, J=6.9 Hz), 4.60-4.85 (2H, m), 5.05-5.30 (1H, m), 7.10-7.60 (10H, m)

MASS (API-ES, Pos): 630.4 (M+H)⁺, 652.2 (M+Na)⁺

### (21) (4R, 9aR)-4-Benzhydryl-2-[(2-cyclopropyl-4-ethoxy-6-methoxy-5-pyrimidinyl)methyl]-8-[(3-methyl-3-oxetanyl)carbonyl]octahydro-2H-pyrazino[1,2-a]pyrazine Dihydrochloride

NMR (CDCl₃, δ): 0.90-1.35 (7H, m), 1.40-1.60 (3H, m), 1.90-4.40 (23H, m), 4.60-4.90 (2H, m), 7.10-7.60 (10H, m)

MASS (API-ES, Pos): 612.4 (M+H)⁺, 634.3 (M+Na)⁺

### (22) (4R, 9aR)-4-Benzhydryl-2-[[2-cyclopropyl-4-(2,2-difluoroethoxy)-6-methoxy-5-pyrimidinyl]methyl]-8-[(3-methyl-3-oxetanyl)carbonyl]octahydro-2H-pyrazino-[1,2-a]pyrazine Dihydrochloride

NMR (CDCl₃, δ): 0.95-1.20 (4H, m), 1.40-1.60 (3H, m), 2.00-2.35 (2H, m), 2.55-2.75 (2H, m), 2.75-3.30 (8H, m), 3.50-3.75 (1H, m), 3.81 (3H, m), 3.90-4.40 (5H, m), 4.40-4.90 (4H, m), 6.10-6.50 (1H, m), 7.10-7.55 (10H, m)

MASS (API-ES, Pos): 648.2 (M+H)⁺, 670.3 (M+Na)⁺

### (23) (4R, 9aR)-4-Benzhydryl-2-[(2-cyclopropyl-4-isopropoxy-6-methoxy-5-pyrimidinyl)methyl]-8-(1,4-dioxan-2-ylcarbonyl)octahydro-2H-pyrazino[1,2-a]pyrazine Dihydrochloride

NMR (CDCl₃, δ): 0.92-1.09 (4H, m), 1.21 (6H, d, J=6.08 Hz), 1.70-2.05 (4H, m), 2.37-3.94 (19H, m), 3.95-4.22 (3H, m), 5.16-5.27 (1H, m), 7.16-7.26 (10H, m)

MASS (ES positive): 642 (M+H)⁺

### (24) (4R, 9aR)-4-Benzhydryl-2-[[2,4-dimethoxy-6-(2,2,2-trifluoroethoxy)-5-pyrimidinyl]methyl]-8-(1,4-dioxan-2-ylcarbonyl)octahydro-2H-pyrazino[1,2-a]pyrazine Dihydrochloride

NMR (CDCl₃, δ): 1.75-1.96 (3H, m), 2.44-2.69 (4H, m), 2.89-3.43 (5H, m), 3.65-3.77 (10H, m), 3.95 (3H, s), 4.10-4.19 (3H, m), 4.56-4.80 (2H, m), 7.12-7.28 (10H, m)

MASS (ES positive): 686 (M+H)⁺

### (25) (4R, 9aR)-4-Benzhydryl-8-(1,4-dioxan-2-ylcarbonyl)-2-[[2-ethoxy-4-methoxy-6-(2,2,2-trifluoroethoxy)-5-pyrimidinyl]methyl]octahydro-2H-pyrazino[1,2-a]pyrazine Dihydrochloride

NMR (CDCl₃, δ): 1.42 (3H, t, J=7.12 Hz), 1.70-2.01 (3H, m), 2.26-3.50 (9H, m), 3.65-3.85 (10H, m), 4.06-4.17 (3H, m), 4.36 (2H, q, J=7.16 Hz), 4.45-4.80 (2H, m), 7.13-7.28 (10H, m)

MASS (ES positive): 672 (M+H)⁺

### (26) (4R, 9aR)-4-Benzhydryl-8-(1,4-dioxan-2-ylcarbonyl)-2-[(2-ethoxy-4-isopropoxy-6-methoxy-5-pyrimidinyl)methyl]octahydro-2H-pyrazino[1,2-a]pyrazine Dihydrochloride

NMR (CDCl₃, δ): 1.25 (6H, d, J=7.10 Hz), 1.41 (3H, t, J=7.06 Hz), 1.70-2.04 (4H, m), 2.37-3.87 (18H, m), 4.10-4.22 (3H, m), 4.35 (2H, q, J=7.08 Hz), 5.22-5.30 (1H, m), 7.13-7.28 (10H, m)

MASS (ES positive): 646 (M+H)⁺

### (27) (4R, 9aS)-4-Benzhydryl-2-[[2-cyclopropyl-4-methoxy-6-(2,2,2-trifluoroethoxy)-5-pyrimidinyl]methyl]-8-[(6-methoxy-2-pyrazinyl)carbonyl]octahydro-2H-pyrazino[1,2-a]pyrazine Dihydrochloride

NMR (CDCl₃, δ): 0.97-1.06 (4H, m), 1.88-2.08 (5H, m), 2.45-2.50 (2H, m), 2.68-3.44 (6H, m), 3.61-3.72 (1H, m), 3.67 and 3.72 (total 3H, each s), 3.77 and 3.91 (total 3H, each s), 4.04-4.15 (1H, m), 4.25-4.71 (3H, m), 7.13-7.28 (10H, m), 8.20 and 8.27 (total 1H, each s), 8.36 and 8.41 (total 1H, each s)

MASS (ES positive): 704 (M+H)⁺

### (28) (4R, 9aR)-4-Benzhydryl-2-[(2-ethoxy-4-isopropoxy-6-methoxy-5-pyrimidinyl)methyl]-8-[(6-methoxy-2-pyrazinyl)carbonyl]octahydro-2H-pyrazino[1,2-a]pyrazine Dihydrochloride

NMR (CDCl₃, δ): 1.16-1.29 (6H, m), 1.37-1.45 (3H, m), 1.85-2.11 (4H, m), 2.45-2.60 (2H, m), 2.70-3.18 (3H, m), 3.20-3.70 (3H, m), 3.68 and 3.70 (total 3H, each s), 3.78 and 3.92 (total 3H, each s), 4.10-4.40 (4H, m), 5.20-5.30 (1H, m), 7.13-7.31 (10H, m), 8.21 and 8.27 (total 1H, each s), 8.36 and 8.42 (total 1H, each s)

MASS (ES positive): 668 (M+H)⁺

### (29) (4R, 9aS)-4-Benzhydryl-2-[[2,4-dimethoxy-6-(2,2,2-trifluoroethoxy)-5-pyrimidinyl]methyl]-8-[(6-methoxy-2-pyrazinyl)carbonyl]octahydro-2H-pyrazino[1,2-a]pyrazine Dihydrochloride

NMR (CDCl₃, δ): 1.88-2.04 (3H, m), 2.44-2.53 (3H, m), 2.71-3.42 (7H, m), 3.65-3.77 (1H, m), 3.77-3.92 (6H, m), 3.92-3.95 (4H, m), 4.05-4.17 (1H, m), 4.25-4.42 (1H, m), 4.57-4.75 (2H, m), 7.16-7.29 (10H, m), 8.21 and 8.27 (total 1H, each s), 8.37 and 8.41 (total 1H, each s)

MASS (ES positive): 694 (M+H)⁺

### (30) (4R, 9aR)-4-Benzhydryl-2-[(2-cyclopropyl-4-isopropoxy-6-methoxy-5-pyrimidinyl)methyl]-8-[(6-methoxy-2-pyrazinyl)carbonyl]octahydro-2H-pyrazino[1,2-a]pyrazine Dihydrochloride

NMR (CDCl₃, δ): 0.85-1.29 (10H, m), 1.85-2.12 (4H, m), 2.50-2.60 (2H, m), 2.68-3.20 (4H, m), 3.25-4.40 (12H, m), 5.15-5.31 (1H, m), 7.13-7.26 (10H, m), 8.21 and 8.27 (total 1H, each s), 8.36 and 8.43 (total 1H, each s)

MASS (ES positive): 664 (M+H)⁺

### (31) (4R, 9aR)-4-Benzhydryl-2-[(2-ethoxy-4-isopropoxy-6-methoxy-5-pyrimidinyl)methyl]-8-[(4-oxido-2-pyrazinyl)carbonyl]octahydro-2H-pyrazino[1,2-a]pyrazine Dihydrochloride

NMR (CDCl₃, δ): 1.18-1.29 (6H, m), 1.36-1.45 (3H, m), 1.85-2.04 (3H, m), 2.45-2.58 (2H, m), 2.72-3.46 (7H, m), 3.69-3.80 (4H, m), 4.10-4.37 (4H, m), 5.20-5.30 (1H, m), 7.17-7.28 (10H, m), 8.02-8.09 (1H, m), 8.32-8.39 (2H, m)

MASS (ES positive): 654 (M+H)⁺

### (32) (4R, 9aR)-4-Benzhydryl-2-[[2-cyclopropyl-4-methoxy-6-(2,2,2-trifluoroethoxy)-5-pyrimidinyl]methyl]-8-[(4-oxido-2-pyrazinyl)carbonyl]octahydro-2H-pyrazino-[1,2-a]pyrazine Dihydrochloride

NMR (CDCl₃, δ): 0.96-1.07 (4H, m), 1.86-2.04 (3H, m), 2.44-2.55 (2H, m), 2.71-3.43 (7H, m), 3.69 and 3.73 (total 3H, each s), 3.73-3.84 (1H, m), 4.10 (2H, t, J=7.18 Hz), 4.20-4.33 (1H, m), 4.49-4.75 (2H, m), 7.19-7.28 (10H, m), 8.02-8.09 (1H, m), 8.31-8.39 (2H, m)

MASS (ES positive): 690 (M+H)⁺

### (33) N-[2-[(6R, 9aR)-6-Benzhydryl-8-[[2-cyclopropyl-4-(2,2-difluoroethoxy)-6-methoxy-5-pyrimidinyl]methyl]-octahydro-2H-pyrazino[1,2-a]pyrazin-2-yl]-1-(hydroxymethyl)-2-oxoethyl]acetamide Dihydrochloride

NMR (CDCl₃, δ): 0.95-1.05 (4H, m), 1.72-2.04 (6H, m), 2.25-2.52 (2H, m), 2.52-2.81 (2H, m), 2.81-3.82 (13H, m), 4.06-4.55 (4H, m), 4.79-4.89 (1H, m), 5.63-6.21 (1H, m), 6.66-6.76 (1H, m), 7.13-7.26 (10H, m)

MASS (ES positive): 679 (M+H)⁺

### (34) N-[1-[[(6R, 9aR)-6-Benzhydryl-8-[[2-cyclopropyl-4-(2,2-difluoroethoxy)-6-methoxy-5-pyrimidinyl]methyl]-octahydro-2H-pyrazino[1,2-a]pyrazin-2-yl]carbonyl]-2-hydroxypropyl]acetamide Dihydrochloride

NMR (CDCl₃, δ): 0.95-1.13 (7H, m), 1.70-2.08 (6H, m), 2.22-3.53 (8H, m), 3.62-3.80 (4H, m), 3.86-4.51 (8H, m), 4.66-4.74 (1H, m), 5.63-6.23 (1H, m), 6.24-6.34 (1H, m), 7.16-7.26 (10H, m)

MASS (ES positive): 693 (M+H)⁺

### (35) 2-[(6R, 9aR)-6-Benzhydryl-8-[(2-cyclopropyl-4-isopropoxy-6-methoxy-5-pyrimidinyl)methyl]octahydro-2H-pyrazino[1,2-a]pyrazin-2-yl]-N,N-dimethyl-2-oxoethanamine Trihydrochloride

IR (KBr): 3490, 1656, 1565, 1448 cm⁻¹

NMR (DMSO-d₆, δ): 1.02-1.50 (9H, m), 2.00-4.60 (28H, m), 5.10-5.18 (1H, m), 7.19-7.41 (10H, m), 9.78 (1H, br)

MASS (API-ES, positive): 613 (M+H)⁺

### (36) 2-[(6R, 9aR)-6-Benzhydryl-8-[(2-ethoxy-4-isopropoxy-6-methoxy-5-pyrimidinyl)methyl]octahydro-2H-pyrazino-[1,2-a]pyrazin-2-yl]-N,N-dimethyl-2-oxoethanamine Trihydrochloride

IR (KBr): 3432, 1666, 1598, 1571 cm⁻¹

NMR (DMSO-d₆, δ): 1.02-1.24 (6H, m), 1.33 (3H, t, J=7.0 Hz), 2.01-2.26 (1H, m), 2.60-4.60 (19H, m), 3.73 (3H, s), 3.78 (3H, s), 4.42 (2H, q, J=7.0 Hz), 5.10-5.18 (1H, m), 7.19-7.41 (10H, m), 9.80 (1H, br)

MASS (API-ES, positive): 617 (M+H)⁺

### (37) 2-[(6R, 9aR)-6-Benzhydryl-8-[[4,6-dimethoxy-2-(2,2,2-trifluoroethoxy)-5-pyrimidinyl]methyl]octahydro-2H-pyrazino[1,2-a]pyrazin-2-yl]-2-oxoethanol Dihydrochloride

NMR (DMSO-d₆, δ): 3.82 (6H, s), 2.10-4.90 (18H, m), 5.06 (2H, q, J=8.9 Hz), 7.10-7.50 (10H, m)

MASS (API-ES): 616 (M+H)⁺(free)

### (38) 2-[(6R, 9aR)-6-Benzhydryl-8-[[2-cyclopropyl-4-(2,2-difluoroethoxy)-6-methoxy-5-pyrimidinyl]methyl]-octahydro-2H-pyrazino[1,2-a]pyrazin-2-yl]-2-oxoethanol Dihydrochloride

NMR (DMSO-d₆, δ): 1.00-1.20 (4H, m), 1.95-2.15 (1H, m), 4.51 (2H, dt, J=3.2, 14 Hz), 2.20-4.90 (21H, m), 6.36 (1H, tt, J=3.2, 55 Hz), 7.10-7.50 (10H, m)

MASS (API-ES): 608 (M+H)⁺(free)

### (39) 2-[(6R, 9aR)-6-Benzhydryl-8-[[4-isopropoxy-6-methoxy-2-(2,2,2-trifluoroethoxy)-5-pyrimidinyl]methyl]octahydro-2H-pyrazino[1,2-a]pyrazin-2-yl]-2-oxoethanol Dihydrochloride

NMR (DMSO-d₆, δ): 1.10-1.40 (6H, m), 4.08 (3H, s), 5.04 (2H, q, J=8.9 Hz), 5.11-5.30 (1H, m), 2.30-5.50 (18H, m), 7.10-7.50 (10H, m)

MASS (API-ES): 644 (M+H)⁺(free)

### (40) 2-[(6R, 9aR)-6-Benzhydryl-8-[[2-cyclopropyl-4-methoxy-6-(2,2,2-trifluoroethoxy)-5-pyrimidinyl]methyl]-octahydro-2H-pyrazino[1,2-a]pyrazin-2-yl]-2-oxoethanol Dihydrochloride

NMR (DMSO-d₆, δ): 1.00-1.20 (4H, m), 1.95-2.15 (1H, m), 2.20-4.80 (21H, m), 4.92 (2H, q, J=8.9 Hz), 7.10-7.50 (10H, m)

MASS (API-ES): 626 (M+H)⁺(free)

### (41) 2-[(6R, 9aR)-6-Benzhydryl-8-[[2,4-dimethoxy-6-(2,2,2-trifluoroethoxy)-5-pyrimidinyl]methyl]octahydro-2H-pyrazino[1,2-a]pyrazin-2-yl]-2-oxoethanol Dihydrochloride

NMR (DMSO-d₆, δ): 3.78 (3H, s), 3.94 (3H, s), 4.95 (2H, q, J=8.9 Hz), 2.20-5.20 (18H, m), 7.10-7.50 (10H, m)

MASS (API-ES): 616 (M+H)⁺(free)

### (42) 2-[(6R, 9aR)-6-Benzhydryl-8-[[4-(2-fluoroethoxy)-2,6-dimethoxy-5-pyrimidinyl]methyl]octahydro-2H-pyrazino[1,2-a]pyrazin-2-yl]-2-oxoethanol Dihydrochloride

IR (KBr): 3394, 2802-2015, 1153, 1088, 1053 cm⁻¹

NMR (DMSO-d₆-D₂O, δ): 2.15-4.80 (28H, m), 7.16-7.46 (10H, m)

MASS (ES+): 580 (M+H)⁺(free)

### (43) 2-[(6R, 9aR)-6-Benzhydryl-8-[[2-(2-fluoroethoxy)-4-methoxy-6-(2,2,2-trifluoroethoxy)-5-pyrimidinyl]-methyl]octahydro-2H-pyrazino[1,2-a]pyrazin-2-yl]-2-oxoethanol Dihydrochloride

IR (KBr): 3433, 2775-2027, 1149, 1092 cm⁻¹

NMR (DMSO-d₆-D₂O, δ): 2.12-4.34 (20H, m), 4.54-5.05 (6H, m), 7.14-7.48 (10H, m)

MASS (ES+): 648 (M+H)⁺(free)

### (44) 2-[(6R, 9aR)-6-Benzhydryl-8-[[2-cyclopropyl-4-(2-fluoroethoxy)-6-methoxy-5-pyrimidinyl]methyl]octahydro-2H-pyrazino[1,2-a]pyrazin-2-yl]-2-oxoethanol Dihydrochloride

NMR (CDCl₃, δ): 0.94-1.06 (4H, m), 1.92-2.04 (4H, m), 2.41-3.46 (11H, m), 3.70 (3H, s), 4.06-4.13 (4H, m), 4.45-4.72 (4H, m), 7.13-7.27 (10H, m)

MASS (ES positive): 590 (M+H)⁺

### (45) 2-[(6R, 9aR)-6-Benzhydryl-8-[[4-(2,2-difluoroethoxy)-2-ethoxy-6-methoxy-5-pyrimidinyl]methyl]octahydro-2H-pyrazino[1,2-a]pyrazin-2-yl]-2-oxoethanol Dihydrochloride

IR (KBr): 3430, 1656, 1606, 1571 cm⁻¹

NMR (DMSO-d₆, δ): 1.33 (3H, t, J=7.0 Hz), 2.10-4.60 (24H, m), 6.08-6.60 (1H, m), 7.22-7.41 (10H, m), 10.00-11.50 (1H, br)

MASS (APCI, Pos): 612 (M+H)

### (46) 2-[(6R, 9aR)-6-Benzhydryl-8-[[2-ethoxy-4-methoxy-6-(2,2,2-trifluoroethoxy)-5-pyrimidinyl]methyl]octahydro-2H-pyrazino[1,2-a]pyrazin-2-yl]-2-oxoethanol Dihydrochloride

IR (KBr): 3459, 1644, 1608, 1430 cm⁻¹

NMR (DMSO-d₆, δ): 1.34 (3H, t, J=7.0 Hz), 2.10-4.50 (20H, m), 4.37 (2H, q, J=7.0 Hz), 4.88-5.00 (2H, m), 7.22-7.41 (10H, m), 10.00-11.50 (1H, br)

MASS (APCI, Pos): 630 (M+H)

### (47) (4R, 9aR)-4-Benzhydryl-2-[[2-cyclopropyl-4-(2,2-difluoroethoxy)-6-methoxy-5-pyrimidinyl]methyl]-8-(methoxyacetyl)octahydro-2H-pyrazino[1,2-a]pyrazine Dihydrochloride

NMR (DMSO-d₆, δ): 0.95-1.10 (4H, m), 1.95-2.12 (1H, m), 3.24 (3H, s), 3.77 (3H, s), 4.51 (2H, t, J=15 Hz), 2.15-4.70 (17H, m), 6.35 (1H, tt, J=3.4, 55 Hz), 7.10-7.50 (10H, m)

MASS (API-ES): 622 (M+H)⁺(free)

### (48) (4R, 9aR)-4-Benzhydryl-2-[[2,4-dimethoxy-6-(2,2,2-trifluoroethoxy)-5-pyrimidinyl]methyl]-8-(methoxyacetyl)octahydro-2H-pyrazino[1,2-a]pyrazine Dihydrochloride

IR (KBr): 3433, 2788-2002, 1147 cm⁻¹

NMR (DMSO-d₆-D₂O, δ): 2.15-5.05 (28H, m), 7.15-7.46 (10H, m)

MASS (ES+): 630 (M+H)⁺(free)

### (49) 3-[(6R, 9aR)-6-Benzhydryl-8-[[2-(2,2-difluoroethoxy)-4-isopropoxy-6-methoxy-5-pyrimidinyl]methyl]octahydro-2H-pyrazino[1,2-a]pyrazin-2-yl]-3-oxo-1-propanol Dihydrochloride

NMR (DMSO-d₆, δ): 1.15-1.30 (6H, m), 4.60 (2H, dt, J=3.3, 15 Hz), 2.20-4.80 (23H, m), 5.10-5.25 (1H, m), 6.41 (1H, tt, J=3.3, 55 Hz), 7.10-7.50 (10H, m)

MASS (API-ES): 640 (M+H)⁺(free)

### (50) 3-[(6R, 9aR)-6-Benzhydryl-8-[[2-ethoxy-4-methoxy-6-(2,2,2-trifluoroethoxy)-5-pyrimidinyl]methyl]octahydro-2H-pyrazino[1,2-a]pyrazin-2-yl]-3-oxo-1-propanol Dihydrochloride

NMR (DMSO-d₆, δ): 1.34 (3H, t, J=7.0 Hz), 4.37 (2H, q, J=7.0 Hz), 2.20-4.60 (23H, m), 4.93 (2H, q, J=8.8 Hz), 7.10-7.50 (10H, m)

MASS (API-ES): 644 (M+H)⁺(free)

### (51) 3-[(6R, 9aR)-6-Benzhydryl-8-[[2-cyclopropyl-4-methoxy-6-(2,2,2-trifluoroethoxy)-5-pyrimidinyl]methyl]octahydro-2H-pyrazino[1,2-a]pyrazin-2-yl]-3-oxo-1-propanol Dihydrochloride

NMR (DMSO-d₆, δ): 1.00-1.20 (4H, m), 1.95-2.15 (1H, m), 2.20-4.70 (23H, m), 4.93 (2H, q, J=8.9 Hz), 7.10-7.50 (10H, m)

MASS (API-ES): 640 (M+H)⁺(free)

### (52) 3-[(6R, 9aR)-6-Benzhydryl-8-[[2-cyclopropyl-4-(2,2-difluoroethoxy)-6-methoxy-5-pyrimidinyl]methyl]-octahydro-2H-pyrazino[1,2-a]pyrazin-2-yl]-3-oxo-1-propanol Dihydrochloride

NMR (DMSO-d₆, δ): 1.00-1.20 (4H, m), 1.95-2.15 (1H, m), 4.52 (2H, dt, J=3.5, 14 Hz), 2.20-4.80 (23H, m), 6.36 (1H, tt, J=3.5, 55 Hz), 7.10-7.50 (10H, m)

MASS (API-ES): 622 (M+H)⁺(free)

### (53) 3-[(6R, 9aR)-6-Benzhydryl-8-[[2-cyclopropyl-4-(2-fluoroethoxy)-6-methoxy-5-pyrimidinyl]methyl]octahydro-2H-pyrazino[1,2-a]pyrazin-2-yl]-3-oxo-1-propanol Dihydrochloride

NMR (CDCl₃, δ): 0.94-1.06 (4H, m), 1.85-2.04 (4H, m), 2.31-3.11 (7H, m), 3.28-3.47 (6H, m), 3.96 (3H, s), 3.79-3.84 (2H, m), 4.06-4.72 (6H, m), 7.13-7.27 (10H, m)

MASS (ES positive): 604 (M+H)⁺

### (54) 3-[(6R, 9aR)-6-Benzhydryl-8-[[2,4-dimethoxy-6-(2,2,2-trifluoroethoxy)-5-pyrimidinyl]methyl]octahydro-2H-pyrazino[1,2-a]pyrazin-2-yl]-3-oxo-1-propanol Dihydrochloride

IR (KBr): 3409, 2790-2046, 1147, 1086 cm⁻¹

NMR (DMSO-d₆-D₂O, δ): 2.05-5.10 (27H, m), 7.06-7.50 (10H, m)

MASS (ES+): 630 (M+H)⁺(free)

### (55) 3-[(6R, 9aR)-6-Benzhydryl-8-[[4-(2,2-difluoroethoxy)-2,6-dimethoxy-5-pyrimidinyl]methyl]octahydro-2H-pyrazino[1,2-a]pyrazin-2-yl]-3-oxo-1-propanol Dihydrochloride

IR (KBr): 3409, 2790-2017, 1159, 1124, 1078 cm⁻¹

NMR (DMSO-d₆-D₂O, δ): 2.05-4.70 (28H, m), 6.31 (pseudo triplet, J=54.5 Hz), 7.10-7.50 (10H, m)

MASS (ES+): 612 (M+H)⁺(free)

### (56) 3-[(6R, 9aR)-6-Benzhydryl-8-[[4-(2-fluoroethoxy)-2,6-dimethoxy-5-pyrimidinyl]methyl]octahydro-2H-pyrazino[1,2-a]pyrazin-2-yl]-3-oxo-1-propanol Dihydrochloride

IR (KBr): 3419, 2788-2015, 1153, 1115, 1086 cm⁻¹

NMR (DMSO-d₆-D₂O, δ): 2.16-4.82 (30H, m), 7.18-7.46 (10H, m)

MASS (ES+): 594 (M+H)⁺(free)

### (57) 3-[(6R, 9aR)-6-Benzhydryl-8-[[2-(2-fluoroethoxy)-4-methoxy-6-(2,2,2-trifluoroethoxy)-5-pyrimidinyl]-methyl]octahydro-2H-pyrazino[1,2-a]pyrazin-2-yl]-3-oxo-1-propanol Dihydrochloride

IR (KBr): 3419, 2802-2015, 1149, 1091, 1047 cm⁻¹

NMR (DMSO-d₆-D₂O, δ): 2.16-5.02 (28H, m), 7.16-7.46 (10H, m)

MASS (ES+): 662 (M+H)⁺(free)

### (58) 2-[(6R, 9aR)-6-Benzhydryl-8-[[2-ethoxy-4-methoxy-6-(2,2,2-trifluoroethoxy)-5-pyrimidinyl]methyl]octahydro-2H-pyrazino[1,2-a]pyrazin-2-yl]-2-oxoethanol

FTIR (KBr): 3438, 1654, 1612, 1571 cm⁻¹

NMR (DMSO-d₆, δ): 1.33 (3H, t, J=7.0 Hz), 2.10-4.60 (26H, m), 6.08-6.60 (1H, m), 7.22-7.36 (10H, m), 10.00-11.50 (1H, br)

MASS (APCI, Pos): 626 (M+H)

### (59) (4R, 9aR)-8-Acetyl-4-benzhydryl-2-[(2-ethoxy-4-isopropoxy-6-methoxy-5-pyrimidinyl)methyl]octahydro-2H-pyrazino[1,2-a]pyrazine Dihydrochloride

NMR (DMSO-d₆, δ): 1.10-1.25 (6H, m), 1.33 (3H, t, J=7.0 Hz), 1.95 (3H, s), 4.33 (2H, q, J=7.0 Hz), 2.20-4.80 (18H, m), 5.05-5.25 (1H, m), 7.10-7.50 (10H, m)

MASS (API-ES): 574 (M+H)⁺(free)

### (60) (4R, 9aR)-8-Acetyl-4-benzhydryl-2-[(2-cyclopropyl-4-isopropoxy-6-methoxy-5-pyrimidinyl)methyl]octahydro-2H-pyrazino[1,2-a]pyrazine Dihydrochloride

NMR (DMSO-d₆, δ): 0.95-1.25 (10H, m), 1.95 (3H, s), 1.90-2.10 (1H, m), 2.20-4.80 (18H, m), 5.05-5.25 (1H, m), 7.10-7.50 (10H, m)

MASS (API-ES): 570 (M+H)⁺(free)

### (61) (4R, 9aR)-4-Benzhydryl-2-[[2-(2,2-difluoroethoxy)-4-isopropoxy-6-methoxy-5-pyrimidinyl]methyl]-8-(3-methoxypropanoyl)octahydro-2H-pyrazino[1,2-a]pyrazine Dihydrochloride

NMR (DMSO-d₆, δ): 1.10-1.30 (6H, m), 4.60 (2H, dt, J=3.3, 15 Hz), 2.20-4.85 (25H, m), 5.05-5.30 (1H, m), 6.42 (1H, tt, J=3.3, 54 Hz), 7.10-7.50 (10H, m)

MASS (API-ES): 654 (M+H)⁺(free)

### (62) (4R, 9aR)-4-Benzhydryl-2-[[4-isopropoxy-6-methoxy-2-(2,2,2-trifluoroethoxy)-5-pyrimidinyl]methyl]-8-(3-methoxypropanoyl)octahydro-2H-pyrazino[1,2-a]pyrazine Dihydrochloride

NMR (DMSO-d₆, δ): 1.10-1.30 (6H, m), 2.20-4.70 (25H, m), 5.03 (2H, q, J=9.0 Hz), 5.10-5.30 (1H, m), 7.10-7.50 (10H, m)

MASS (API-ES): 672 (M+H)⁺(free)

### (63) (4R, 9aR)-4-Benzhydryl-2-[[2-(2,2-difluoroethoxy)-4-ethoxy-6-methoxy-5-pyrimidinyl]methyl]-8-(3-methoxypropanoyl)octahydro-2H-pyrazino[1,2-a]pyrazine Dihydrochloride

NMR (DMSO-d₆, δ): 1.21 (3H, t, J=6.9 Hz), 3.78 (3H, s), 4.23 (2H, q, J=6.9 Hz), 1.80-4.50 (22H, m), 4.57 (2H, dt, J=3.5, 15 Hz), 6.41 (1H, tt, J=3.5, 55 Hz), 7.10-7.50 (10H, m)

MASS (API-ES): 640 (M+H)⁺(free)

### (64) (4R, 9aR)-4-Benzhydryl-2-[[4-(2,2-difluoroethoxy)-2,6-dimethoxy-5-pyrimidinyl]methyl]-8-(3-methoxypropanoyl)-octahydro-2H-pyrazino[1,2-a]pyrazine Dihydrochloride

NMR (DMSO-d₆, δ): 3.93 (3H, s), 4.54 (2H, t, J=14 Hz), 2.10-4.90 (25H, m), 6.39 (1H, t, J=55 Hz), 7.10-7.50 (10H, m)

MASS (API-ES): 626 (M+H)⁺(free)

### (65) (4R, 9aR)-4-Benzhydryl-2-[[2-ethoxy-4-methoxy-6-(2,2,2-trifluoroethoxy)-5-pyrimidinyl]methyl]-8-(3-methoxypropanoyl)octahydro-2H-pyrazino[1,2-a]pyrazine Dihydrochloride

NMR (DMSO-d₆, δ): 1.34 (3H, t, J=7.1 Hz), 4.38 (2H, q, J=7.1 Hz), 4.94 (2H, q, J=8.6 Hz), 2.20-5.00 (25H, m), 7.10-7.50 (10H, m)

MASS (API-ES): 658 (M+H)⁺(free)

### (66) (4R, 9aR)-4-Benzhydryl-2-[[4-(2,2-difluoroethoxy)-2-ethoxy-6-methoxy-5-pyrimidinyl]methyl]-8-(3-methoxypropanoyl)octahydro-2H-pyrazino[1,2-a]pyrazine Dihydrochloride

NMR (DMSO-d₆, δ): 1.34 (3H, t, J=7.1 Hz), 4.36 (2H, q, J=7.1 Hz), 4.52 (2H, t, J=15 Hz), 2.20-5.00 (25H, m), 6.38 (1H, t, J=55 Hz), 7.10-7.50 (10H, m)

MASS (API-ES): 640 (M+H)⁺(free)

### (67) (4R, 9aR)-4-Benzhydryl-2-[[2,4-dimethoxy-6-(2,2,2-trifluoroethoxy)-5-pyrimidinyl]methyl]-8-(3-methoxypropanoyl)octahydro-2H-pyrazino[1,2-a]pyrazine Dihydrochloride

NMR (DMSO-d₆, δ): 3.94 (3H, s), 2.20-4.70 (25H, m), 4.95 (2H, q, J=8.8 Hz), 7.10-7.50 (10H, m)

MASS (API-ES): 644 (M+H)⁺(free)

### (68) (4R, 9aR)-4-Benzhydryl-2-[[2-cyclopropyl-4-(2,2-difluoroethoxy)-6-methoxy-5-pyrimidinyl]methyl]-8-(3-methoxypropanoyl)octahydro-2H-pyrazino[1,2-a]pyrazine Dihydrochloride

NMR (DMSO-d₆, δ): 0.95-1.10 (4H, m), 1.95-2.15 (1H, m), 4.52 (2H, dt, J=3.6, 15 Hz), 2.20-4.80 (25H, m), 6.35 (1H, tt, J=3.6, 55 Hz), 7.10-7.50 (10H, m)

MASS (API-ES): 636 (M+H)⁺(free)

### (69) (4R, 9aR)-4-Benzhydryl-2-[[2-cyclopropyl-4-methoxy-6-(2,2,2-trifluoroethoxy)-5-pyrimidinyl]methyl]-8-(3-methoxypropanoyl)octahydro-2H-pyrazino[1,2-a]pyrazine Dihydrochloride

NMR (DMSO-d₆, δ): 1.00-1.20 (4H, m), 1.95-2.15 (1H, m), 4.93 (2H, q, J=8.8 Hz), 2.20-5.20 (25H, m), 7.10-7.50 (10H, m)

MASS (APCI): 654 (M+H)⁺(free)

### (70) (4R, 9aR)-4-Benzhydryl-2-[[2-(2-fluoroethoxy)-4-methoxy-6-(2,2,2-trifluoroethoxy)-5-pyrimidinyl]methyl]-8-(3-methoxypropanoyl)octahydro-2H-pyrazino[1,2-a]pyrazine Dihydrochloride

NMR (DMSO-d₆-D₂O, δ): 1.84-5.10 (31H, m), 7.10-7.55 (10H, m)

MASS (ES+): 698 (M+Na)⁺, 676 (M+H)⁺(free)

### (71) (4R, 9aR)-4-Benzhydryl-2-[(2-ethoxy-4-isopropoxy-6-methoxy-5-pyrimidinyl)methyl]-8-(2-pyrazinylcarbonyl)-octahydro-2H-pyrazino[1,2-a]pyrazine Dihydrochloride

NMR (DMSO-d₆, δ): 1.00-4.40 (9H, m), 3.77 (3H, s), 2.20-4.80 (17H, m), 5.00-5.20 (1H, m), 7.10-7.50 (10H, m), 8.60-8.90 (3H, m)

MASS (API-ES): 638 (M+H)⁺(free)

### (72) (4R, 9aR)-4-Benzhydryl-2-[(2-cyclopropyl-4-isopropoxy-6-methoxy-5-pyrimidinyl)methyl]-8-(2-pyrazinylcarbonyl)-octahydro-2H-pyrazino[1,2-a]pyrazine Dihydrochloride

NMR (DMSO-d₆, δ): 0.95-1.25 (10H, m), 1.90-2.10 (1H, m), 3.78 (3H, s), 2.20-4.70 (15H, m), 5.00-5.25 (1H, m), 7.10-7.50 (10H, m), 8.60-8.85 (3H, m)

MASS (API-ES): 634 (M+H)⁺(free)

### (73) (4R, 9aR)-4-Benzhydryl-2-[[4-(2,2-difluoroethoxy)-2,6-dimethoxy-5-pyrimidinyl]methyl]-8-(2-pyrazinylcarbonyl)octahydro-2H-pyrazino[1,2-a]pyrazine Dihydrochloride

NMR (DMSO-d₆, δ): 3.78 (3H, s), 3.93 (3H, s), 2.20-4.80 (17H, m), 6.00-6.70 (1H, m), 7.10-7.50 (10H, m), 8.60-8.90 (3H, m)

MASS (API-ES): 646 (M+H)⁺(free)

### (74) (4R, 9aR)-4-Benzhydryl-2-[[2-(2,2-difluoroethoxy)-4-isopropoxy-6-methoxy-5-pyrimidinyl]methyl]-8-(2-pyrazinylcarbonyl)octahydro-2H-pyrazino[1,2-a]pyrazine Dihydrochloride

NMR (DMSO-d₆, δ): 1.05-1.30 (6H, m), 3.80 (3H, s), 4.60 (2H, t, J=15 Hz), 2.20-4.80 (15H, m), 5.00-5.30 (1H, m), 6.42 (1H, t, J=52 Hz), 7.10-7.50 (10H, m), 8.60-8.90 (3H, m)

MASS (API-ES): 674 (M+H)⁺(free)

### (75) (4R, 9aR)-4-Benzhydryl-2-[[2,4-dimethoxy-6-(2,2,2-trifluoroethoxy)-5-pyrimidinyl]methyl]-8-(2-pyrazinylcarbonyl)octahydro-2H-pyrazino[1,2-a]pyrazine Dihydrochloride

NMR (DMSO-d₆, δ): 3.80 (3H, s), 2.20-4.75 (18H, m), 4.80-5.00 (2H, m), 7.10-7.50 (10H, m), 8.63 (1H, d, J=11.3 Hz), 8.75 (1H, d, J=10.1 Hz), 8.83 (1H, d, J=8.0 Hz)

MASS (API-ES): 664 (M+H)⁺(free)

### (76) (4R, 9aR)-4-Benzhydryl-2-[[2-(2,2-difluoroethoxy)-4-ethoxy-6-methoxy-5-pyrimidinyl]methyl]-8-(2-pyrazinylcarbonyl)octahydro-2H-pyrazino[1,2-a]pyrazine Dihydrochloride

NMR (DMSO-d₆, δ): 1.10-1.25 (3H, m), 3.81 (3H, s), 4.61 (2H, t, J=15 Hz), 2.20-4.80 (17H, m), 6.43 (1H, t, J=54 Hz), 7.10-7.50 (10H, m), 8.60-8.90 (3H, m)

MASS (API-ES): 660 (M+H)⁺(free)

### (77) (4R, 9aR)-4-Benzhydryl-2-[[2-ethoxy-4-methoxy-6-(2,2,2-triifluoroethoxy)-5-pyrimidinyl]methyl]-8-(2-pyrazinylcarbonyl)octahydro-2H-pyrazino[1,2-a]pyrazine Dihydrochloride

NMR (DMSO-d₆, δ): 1.34 (3H, t, J=7.1 Hz), 3.79 (3H, s), 2.20-4.75 (17H, m), 4.80-5.05 (2H, m), 7.10-7.50 (10H, m), 8.60-8.90 (3H, m)

MASS (API-ES): 678 (M+H)⁺(free)

### (78) (4R, 9aR)-4-Benzhydryl-2-[(2-cyclopropyl-4-ethoxy-6-methoxy-5-pyrimidinyl)methyl]-8-(2-pyrazinylcarbonyl)-octahydro-2H-pyrazino[1,2-a]pyrazine Dihydrochloride

NMR (DMSO-d₆, δ): 1.00-1.25 (7H, m), 1.90-2.10 (1H, m), 3.78 (3H, s), 2.20-4.60 (17H, m), 7.10-7.50 (10H, m), 8.60-8.90 (3H, m)

MASS (API-ES): 620 (M+H)⁺(free)

### (79) (4R, 9aR)-4-Benzhydryl-2-[(2,4-diethoxy-6-methoxy-5-pyrimidinyl)methyl]-8-(2-pyrazinylcarbonyl)-octahydro-2H-pyrazino[1,2-a]pyrazine Dihydrochloride

NMR (DMSO-d₆, δ): 1.10-1.40 (6H, m), 3.78 (3H, s), 2.20-4.70 (19H, m), 7.10-7.50 (10H, m), 8.60-8.90 (3H, m)

MASS (API-ES): 624 (M+H)⁺(free)

### (80) (4R, 9aR)-4-Benzhydryl-2-[[4-(2,2-difluoroethoxy)-2-ethoxy-6-methoxy-5-pyrimidinyl]methyl]-8-(2-pyrazinylcarbonyl)octahydro-2H-pyrazino[1,2-a]pyrazine Dihydrochloride

NMR (DMSO-d₆, δ): 1.30-1.40 (3H, m), 3.77 (3H, s), 2.20-4.80 (19H, m), 6.00-6.70 (1H, m), 7.10-7.50 (10H, m), 8.60-8.90 (3H, m)

MASS (API-ES): 682 (M+Na)⁺(free)

### (81) (4R, 9aR)-4-Benzhydryl-2-[[2-cyclopropyl-4-methoxy-6-(2,2,2-trifluoroethoxy)-5-pyrimidinyl]methyl]-8-(2-pyrazinylcarbonyl)octahydro-2H-pyrazino[1,2-a]pyrazine Dihydrochloride

NMR (DMSO-d₆, δ): 1.00-1.20 (4H, m), 1.95-2.15 (1H, m), 3.79 (3H, s), 2.20-4.60 (15H, m), 4.80-5.05 (2H, m), 7.10-7.50 (10H, m), 8.60-8.90 (3H, m)

MASS (API-ES): 674 (M+H)⁺(free)

### (82) (4R, 9aR)-4-Benzhydryl-2-[[2-cyclopropyl-4-(2,2-difluoroethoxy)-6-methoxy-5-pyrimidinyl]methyl]-8-(2-pyrazinylcarbonyl)octahydro-2H-pyrazino[1,2-a]pyrazine Dihydrochloride

NMR (DMSO-d₆, δ): 1.00-1.20 (4H, m), 1.95-2.15 (1H, m), 3.77 (3H, s), 2.20-4.70 (17H, m), 5.95-6.70 (1H, m), 7.10-7.50 (10H, m), 8.60-8.90 (3H, m)

MASS (API-ES): 656 (M+H)⁺(free)

### (83) (4R, 9aR)-4-Benzhydryl-2-[[2-cyclopropyl-4-(2-fluoroethoxy)-6-methoxy-5-pyrimidinyl]methyl]-8-(2-pyrazinylcarbonyl)octahydro-2H-pyrazino[1,2-a]pyrazine Dihydrochloride

NMR (CDCl₃, δ): 0.95-1.07 (4H, m), 1.89-2.04 (4H, m), 2.40-3.48 (9H, m), 3.60-3.80 (4H, m), 4.07-4.71 (6H, m), 7.16-7.29 (10H, m), 8.45-8.63 (2H, m), 8.86-8.90 (1H, m)

MASS (ES positive): 638 (M+H)⁺

### (84) (4R, 9aR)-4-Benzhydryl-2-[[2-fluoroethoxy)-4-methoxy-6-(2,2,2-trifluoroethoxy)-5-pyrimidinyl]methyl]-8-(2-pyrazinylcarbonyl)octahydro-2H-pyrazino[1,2-a]pyrazine Dihydrochloride

NMR (DMSO-d₆-D₂O, δ): 1.82-5.10 (26H, m), 7.10-7.50 (10H, m), 8.55-8.90 (3H, m)

MASS (ES+): 718 (M+Na)⁺, 696 (M+H)⁺(free)

### (85) (4R, 9aR)-4-Benzhydryl-2-[[4-(2-fluoroethoxy)-2,6-dimethxy-5-pyrimidinyl]methyl]-8-(2-pyrazinylcarbonyl)octahydro-2H-pyrazino[1,2-a]pyrazine Dihydrochloride

NMR (DMSO-d₆-D₂O, δ): 1.85-4.85 (25H, m), 7.10-7.58 (10H, m), 8.55-8.90 (3H, m)

MASS (ES+): 628 (M+H)⁺(free)

### (86) (4R, 9aR)-4-Benzhydryl-2-[[2,4-dimethoxy-6-(2,2,2-trifluoroethoxy)-5-pyrimidinyl]methyl]-8-[(3-ethyl-3-oxetanyl)carbonyl]octahydro-2H-pyrazino[1,2-a]pyrazine Dihydrochloride

NMR (DMSO-d₆, δ): 0.70-0.90 (3H, m), 1.75-4.35 (25H, m), 4.55-5.05 (4H, m), 7.10-7.45 (10H, m)

MASS (API-ES, Pos): 670.2 (M+H)⁺, 692.2 (M+Na)⁺(free)

### (87) (4R, 9aR)-4-Benzhydryl-2-[[2-cyclopropyl-4-methoxy-6-(2,2,2-trifluoroethoxy)-5-primidinyl]methyl]-8-[(3-ethyl-3-oxetanyl)carbonyl]octahydro-2H-pyrazino[1,2-a]-pyrazine Dihydrochloride

NMR (DMSO-d₆, δ): 0.70-0.90 (3H, m), 0.95-1.20 (4H, m), 1.75-4.40 (23H, m), 4.55-5.05 (4H, m), 7.10-7.45 (10H, m)

MASS (API-ES, Pos): 680.3 (M+H)⁺(free)

### (88) (6R, 9aR)-6-Benzhydryl-8-[[2-cyclopropyl-4-methoxy-6-(2,2,2-trifluoroethoxy)-5-pyrimidinyl]methyl]-N,N-dimethyloctahydro-2H-pyrazino[1,2-a]pyrazine-2-carboxamide Dihydrochloride

NMR (DMSO-d₆, δ): 0.95-1.15 (4H, m), 1.90-4.60 (25H, m), 4.80-5.00 (2H, m), 7.10-7.45 (10H, m)

MASS (API-ES, Pos): 639.2 (M+H)⁺, 661.3 (M+Na)⁺(free)

### (89) (6R, 9aR)-6-Benzhydryl-8-[(2-ethoxy-4-isopropoxy-6-methoxy-5-pyrimidinyl)methyl]-N,N-dimethyloctahydro-2H-pyrazino[1,2-a]pyrazine-2-carboxamide Dihydrochloride

NMR (DMSO-d₆, δ): 1.17 (3H, d, J=6.0 Hz), 1.20 (3H, d, J=7.1 Hz), 1.33 (3H, t, J=7.0 Hz), 1.90-4.30 (24H, m), 4.34 (2H, q, J=7.0 Hz), 5.16 (1H, qq, J=6.2, 6.2 Hz), 7.10-7.45 (10H, m)

MASS (API-ES, Pos): 603.3 (M+H)⁺, 625.2 (M+Na)⁺(free)

### (90) (4R, 9aR)-4-Benzhydryl-2-[[2-cyclopropyl-4-methoxy-6-(2,2,2-trifluoroethoxy)-5-pyrimidinyl]methyl]-8-(1,3-dioxan-5-ylcarbonyl)octahydro-2H-pyrazino[1,2-a]-pyrazine Dihydrochloride

NMR (CDCl₃, δ): 1.00-1.15 (4H, m), 1.90-4.40 (24H, m) 4.50-4.65 (1H, m), 4.80-5.05 (3H, m), 7.10-7.50 (10H, m)

MASS (API-ES, Pos): 682.2 (M+H)⁺, 704.3 (M+Na)⁺(free)

### EXAMPLE 9

To a solution of (4R, 9aR)-4-benzhydryl-2-[[2-ethoxy-4-methoxy-6-(2,2,2-trifluoroethoxy)-5-pyrimidinyl]methyl]-8-(trifluoroacetyl)octahydro-2H-pyrazino[1,2-a]pyrazine in 2.3 ml of methanol was added 1.1 ml of 10% potassium carbonate aqueous solution at 0° C., then stirred for 6 hours at ambient temperature. The reaction mixture was evaporated in vacuo, added 5 ml of brine, extracted with dichloromethane (10 ml×3), dried over magnesium sulfate and evaporated to give (4R, 9aS)-4-benzhydryl-2-[[2-ethoxy-4-methoxy-6-(2,2,2-trifluoroethoxy)-5-pyrimidinyl]methyl]octahydro-2H-pyrazino[1,2-a]pyrazine as a pale yellow foam (94.0 mg).

NMR (CDCl₃, δ): 1.42 (3H, t, J=7.1 Hz), 1.70-2.00 (2H, m), 2.25-2.90 (8H, m), 3.20-3.30 (1H, m), 3.44 (2H, s), 3.70 (3H, s), 4.11 (1H, d, J=7.4 Hz), 4.36 (2H, q, J=7.1 Hz), 4.45-4.60 (1H, m), 4.65-4.80 (1H, m), 7.10-7.35 (10H, m)

MASS (API-ES, Pos): 572.3 (M+H)⁺, 594.2 (M+Na)⁺

### EXAMPLE 10

The following compounds were obtained according to a similar manner to that of Example 9.

### (1) (4R, 9aS)-4-Benzhydryl-2-[[2,4-dimethoxy-6-(2,2,2-trifluoroethoxy)-5-pyrimidinyl]methyl]octahydro-2H-pyrazino[1,2-a]pyrazine

NMR (CDCl₃, δ): 1.8-2.0 (3H, m), 2.3-2.9 (8H, m), 3.2-3.35 (1H, m), 3.27 (2H, s), 3.72 (3H, s), 3.94 (3H, s), 4.05-4.15 (1H, m), 4.45-4.80 (2H, m), 7.05-7.35 (10H, m)

MASS (API-ES, Pos): 558.3 (M+H)⁺

### (2) (4R, 9aS)-4-Benzhydryl-2-[(2-cyclopropyl-4-isopropoxy-6-methoxy-5-pyrimidinyl]methyl]octahydro-2H-pyrazino-[1,2-a]pyrazine

NMR (CDCl₃, δ): 0.90-1.00 (2H, m), 1.00-1.10 (2H, m), 1.15-1.25 (6H, m), 1.70-2.05 (4H, m), 2.30-2.90 (8H, m), 3.20-3.35 (1H, m), 3.41 (1H, d, J=13.2 Hz), 3.50 (1H, d, J=13.2 Hz), 3.64 (3H, s), 4.13 (1H, d, J=7.4 Hz), 5.15-5.30 (1H, m), 7.05-7.35 (10H, m)

MASS (API-ES, Pos): 528.4 (M+H)⁺

### EXAMPLE 11

To a solution of (4R, 9aS)-4-benzhydryl-2-[[2-ethoxy-4-methoxy-6-(2,2,2-trifluoroethoxy)-5-pyrimidinyl]methyl]-octahydro-2H-pyrazino[1,2-a]pyrazine (46.9 mg) in dichloromethane (0.4 ml) was added picolinic acid (11.1 mg), 1-hydroxybenzotriazole (11.1 mg) and 1-(3-dimethylaminopropyl)-3-ethylcarbodiimide hydrochloride (23.6 mg), then stirred for 2hours at ambient temperature. The reaction mixture was added 2 ml of saturated sodium hydrogen carbonate aqueous solution, then extracted with dichloromethane (5 ml×3). The organic layer was dried over diatomaceous earth and evaporated under reduced pressure to give a crude oil. Purification by preparative TLC (0.5 mm silica gel, dichloromethane:methanol=10:1) gave pale yellow oil. To a solution of the oil in 2 ml of ethyl acetate was added 0.5 ml of 4N hydrogen chloride in ethyl acetate at 0° C., then the volatiles were evaporated in vacuo to give (4R, 9aR)-4-benzhydryl-2-[[2-ethoxy-4-methoxy-6-(2,2,2-trifluoroethoxy)-5-pyrimidinyl]methyl]-8-(2-pyridinylcarbonyl)octahydro-2H-pyrazino[1,2-a]pyrazine trihydrochloride as a white solid (57.3 mg).

NMR (DMSO-d₆, δ): 1.10-1.40 (3H, m), 1.80-4.50 (20H, m), 4.75-5.05 (2H, m), 7.10-7.65 (12H, m), 7.85-8.00 (1H, m), 8.50-8.65 (1H, m)

MASS (API-ES, Pos): 677.2 (M+H)⁺, 699.2 (M+Na)⁺(free)

### EXAMPLE 12

The following compounds were obtained according to a similar manner to that of Example 11.

### (1) (4R, 9aR)-4-Benzhydryl-2-[[2-ethoxy-4-methoxy-6-(2,2,2-trifluoroethoxy)-5-pyrimidinyl]methyl]-8-(3-pyridinylcarbonyl)octahydro-2H-pyrazino[1,2-a]pyrazine Trihydrochloride

NMR (DMSO-d₆, δ): 1.20-1.40 (3H, m), 2.10-4.50 (20H, m), 4.80-5.05 (2H, m), 7.10-7.45 (10H, m), 7.50-7.70 (1H, m), 7.80-8.00 (1H, m), 8.55-8.80 (2H, m)

MASS (API-ES, Pos): 677.3 (M+H)⁺, 699.2 (M+Na)⁺(free)

### (2) (4R, 9aR)-4-Benzhydryl-2-[[2-ethoxy-4-methoxy-6-(2,2,2-trifluoroethoxy)-5-pyrimidinyl]methyl]-8-isonicotinoyloctahydro-2H-pyrazino[1,2-a]pyrazine Trihydrochloride

NMR (DMSO-d₆, δ): 1.20-1.40 (3H, m), 1.90-4.50 (20H, m), 4.70-5.05 (2H, m), 7.10 (10H, m), 7.62 (2H, s), 8.70-8.90 (2H, m)

MASS (API-ES, Pos): 677.3 (M+H)⁺, 699.2 (M+Na)⁺(free)

### (3) 3-[[(6R, 9aR)-6-Benzhydryl-8-[[2-ethoxy-4-methoxy-6-(2,2,2-trifluoroethoxy)pyrimidin-5-yl]methyl]octahydro-2H-pyrazino[1,2-a]pyrazin-2-yl]carbonyl]pyridin-2-ol Dihydrochloride

NMR (DMSO-d₆, δ): 1.10-1.40 (3H, m), 1.90-4.50 (20H, m), 4.80-5.05 (2H, m), 6.15-6.30 (1H, m), 7.10-7.60 (12H, m), 11.70-12.10 (1H, m)

MASS (API-ES, Pos): 693.2 (M+H)⁺, 715.3 (M+Na)⁺(free)

### (4) (4R, 9aR)-4-Benzhydryl-2-[[2-ethoxy-4-methoxy-6-(2,2,2-trifluoroethoxy)pyrimidin-5-yl]methyl]-8-(pyridin-3-ylacetyl)octahydro-2H-pyrazino[1,2-a]pyrazine Trihydrochloride

NMR (DMSO-d₆, δ): 1.34 (3H, t, J=7.0 Hz), 1.90-4.60 (22H, m), 4.80-5.05 (2H, m), 7.10-7.50 (10H, m), 7.90-8.05 (1H, m), 8.36 (1H, d, J=8.2 Hz), 8.75 (1H, s), 8.81 (1H, d, J=5.5 Hz)

MASS (API-ES, Pos): 691.3 (M+H)⁺, 713.3 (M+Na)⁺(free)

### (5) (4R, 9aR)-4-Benzhydryl-2-[[2-ethoxy-4-methoxy-6-(2,2,2-trifluoroethoxy)pyrimidin-5-yl]methyl]-8-(1H-pyrrol-2-ylcarbonyl)octahydro-2H-pyrazino[1,2-a]pyrazine Dihydrochloride

NMR (DMSO-d₆, δ): 1.33 (3H, t, J=7.0 Hz), 1.90-4.45 (20H, m), 4.75-5.00 (2H, m), 6.05-6.15 (1H, m), 6.48 (1H, s), 6.89 (1H, s), 7.15-7.55 (10H, m), 11.46 (1H, s)

MASS (API-ES, Pos): 665.3 (M+H)⁺, 687.2 (M+Na)⁺(free)

### (6) (4R, 9aR)-4-Benzhydryl-2-[[2-ethoxy-4-methoxy-6-(2,2,2-trifluoroethoxy)pyrimidin-5-yl]methyl]-8-[(1-methyl-1H-pyrrol-2-yl)carbonyl]octahydro-2H-pyrazino[1,2-a]-pyrazine Dihydrochloride

NMR (DMSO-d₆, δ): 1.34 (3H, t, J=7.0 Hz), 1.90-4.50 (23H, m), 4.80-5.05 (2H, m), 6.02 (1H, s), 6.29 (1H, s), 6.91 (1H, s), 7.05-7.55 (10H, m)

MASS (API-ES, Pos): 679.3 (M+H)⁺, 701.2 (M+Na)⁺(free)

### (7) (4R, 9aR)-4-Benzhydryl-2-[[2-ethoxy-4-methoxy-6-(2,2,2-trifluoroethoxy)pyrimidin-5-yl]methyl]-8-(1H-pyrazol-4-ylcarbonyl)octahydro-2H-pyrazino[1,2-a]pyrazine Dihydrochloride

NMR (DMSO-d₆, δ): 1.33 (3H, t, J=7.1 Hz), 1.90-4.35 (18H, m), 4.38 (2H, q, J=7.0 Hz), 4.80-5.05 (2H, m), 7.10-7.45 (10H, m), 7.88 (2H, s)

MASS (API-ES, Pos): 666.2 (M+H)⁺, 688.1 (M+Na)⁺(free)

### (8) (4R, 9aR)-4-Benzhydryl-2-[[2-ethoxy-4-methoxy-6-(2,2,2-trifluoroethoxy)pyrimidin-5-yl]methyl]-8-(1H-imidazol-4-ylcarbonyl)octahydro-2H-pyrazino[1,2-a]pyrazine Trihydrochloride

NMR (DMSO-d₆, δ): 1.33 (3H, t, J=7.0 Hz), 1.90-4.35 (18H, m), 4.37 (2H, q, J=7.0 Hz), 4.70-5.00 (2H, m), 7.10-7.50 (10H, m), 7.99 (1H, m), 8.81 (1H, br s)

MASS (API-ES, Pos): 666.3 (M+H)⁺, 688.3 (M+Na)⁺(free)

### (9) (4R, 9aR)-4-Benzhydryl-2-[[2-ethoxy-4-methoxy-6-(2,2,2-trifluoroethoxy)pyrimidinyl]methyl]-8-[(2R)-tetrahydro-2-furanylcarbonyl]octahydro-2H-pyrazino[1,2-a]pyrazine Dihydrochloride

NMR (DMSO-d₆, δ): 1.34 (3H, t, J=7.0 Hz), 1.70-1.85 (2H, m), 1.85-2.05 (2H, m), 2.10-4.35 (20H, m), 4.38 (2H, q, J=7.0 Hz), 4.55-4.70 (1H, m), 4.80-5.05 (2H, m), 7.10-7.60 (10H, m)

MASS (API-ES, Pos): 670.3 (M+H)⁺, 692.2 (M+Na)⁺(free)

### (10) (4R, 9aR)-4-Benzhydryl-2-[[2-ethoxy-4-methoxy-6-(2,2,2-trifluoroethoxy)-5-pyrimidinyl]methyl]-8-[(2S)-tetrahydro-2-furanylcarbonyl]octahydro-2H-pyrazino-[1,2-a]pyrazine Dihydrochloride

NMR (DMSO-d₆, δ): 1.37 (3H, t, J=7.0 Hz), 1.70-4.35 (24H, m), 4.38 (2H, q, J=7.0 Hz), 4.50-4.65 (1H, m), 4.80-5.05 (2H, m), 7.05-7.55 (10H, m)

MASS (API-ES, Pos): 670.3 (M+H)⁺, 692.3 (M+Na)⁺(free)

### (11) (4R, 9aR)-4-Benzhydryl-2-[[2-ethoxy-4-methoxy-6-(2,2,2-trifluoroethoxy)-5-pyrimidinyl]methyl]-8-(tetrahydro-3-furanylcarbonyl)octahydro-2H-pyrazino[1,2-a]pyrazine Dihydrochloride

NMR (DMSO-d₆, δ): 1.34 (3H, t, J=7.0 Hz), 1.80-4.50 (27H, m), 4.80-5.05 (2H, m), 7.10-7.50 (10H, m)

MASS (API-ES, Pos): 670.3 (M+H)⁺, 692.2 (M+Na)⁺(free)

### (12) (4R, 9aR)-4-Benzhydryl-2-[[2-ethoxy-4-methoxy-6-(2,2,2-trifluoroethoxy)-5-pyrimidinyl]methyl]-8-[(2-methoxy-3-pyridinyl)carbonyl]octahydro-2H-pyrazino[1,2-a]pyrazine Trihydrochloride

NMR (DMSO-d₆, δ): 1.25-1.45 (3H, m), 1.90-4.50 (23H, m), 4.75-5.05 (2H, m), 7.00-7.50 (11H, m), 7.60-7.75 (1H, m), 8.20-8.35 (1H, m)

MASS (API-ES, Pos): 707.2 (M+H)⁺, 729.2 (M+Na)⁺(free)

### (13) (4R, 9aR)-4-Benzhydryl-2-[[2-ethoxy-4-methoxy-6-(2,2,2-trifluoroethoxy)-5-pyrimidinyl]methyl]-8-[(2-ethoxy-3-pyridinyl)carbonyl]octahydro-2H-pyrazino[1,2-a]pyrazine Trihydrochloride

NMR (DMSO-d₆, δ): 1.10-1.45 (6H, m), 1.90-4.95 (22H, m), 4.96 (2H, q, J=8.7 Hz), 6.95-7.10 (1H, m), 7.10-7.50(10H, m), 7.60-7.75 (1H, m), 8.15-8.30 (1H, m)

MASS (API-ES, Pos): 721.3 (M+H)⁺, 743.2 (M+Na)⁺(free)

### (14) (4R, 9aR)-4-Benzhydryl-8-[(2,6-dimethoxy-3-pyridinyl)carbonyl]-2-[[2-ethoxy-4-methoxy-6-(2,2,2-trifluoroethoxy)-5-pyrimidinyl]methyl]octahydro-2H-pyrazino[1,2-a]pyrazine Trihydrochloride

NMR (DMSO-d₆, δ): 1.25-1.45 (3H, m), 1.90-4.60 (26H, m), 4.75-5.10 (2H, m), 6.35-6.55 (1H, m), 7.10-7.50 (10H, m), 7.50-7.30 (1H, m)

MASS (API-ES, Pos): 737.2 (M+H)⁺, 759.2 (M+Na)⁺(free)

### (15) 3-[[(6R, 9aR)-6-Benzhydryl-8-[[2-ethoxy-4-methoxy-6-(2,2,2-trifluoroethoxy)-5-pyrimidinyl]methyl]octahydro-2H-pyrazino[1,2-a]pyrazin-2-yl]carbonyl]-2-pyrazinamine Trihydrochloride

NMR (DMSO-d₆, δ): 1.25-1.45 (3H, m), 1.90-4.50 (20H, m), 4.75-5.05 (2H, m), 7.15-7.50 (10H, m), 7.65-7.80 (1H, m), 7.95-8.10 (1H, m)

MASS (API-ES, Pos): 693.2 (M+H)⁺, 715.3 (M+Na)⁺(free)

### (16) 3-[[(6R, 9aR)-6-Benzhydryl-8-[[2-ethoxy-4-methoxy-6-(2,2,2-trifluoroethoxy)-5-pyrimidinyl]methyl]octahydro-2H-pyrazino[1,2-a]pyrazin-2-yl]carbonyl]-2-pyrazinecarboxamide Dihydrochloride

NMR (DMSO-d₆, δ): 1.25-1.45 (3H, m), 1.90-4.50 (20H, m), 4.75-5.10 (2H, m), 7.00-7.45 (10H, m), 7.50-7.90 (1H, m), 8.20-8.45 (1H, m), 8.65-8.85 (2H, m)

MASS (API-ES, Pos): 721.3 (M+H)⁺, 743.2 (M+Na)⁺(free)

### (17) (4R, 9aR)-4-Benzhydryl-2-[[2-ethoxy-4-methoxy-6-(2,2,2-trifluoroethoxy)-5-pyrimidinyl]methyl]-8-[(5-methyl-2-pyrazinyl)carbonyl]octahydro-2H-pyrazino[1,2-a]pyrazine Dihydrochloride

NMR (DMSO-d₆, δ): 1.25-1.40 (3H, m), 1.90-4.50 (23H, m), 4.75-5.05 (2H, m), 7.10-7.50 (10H, m), 8.45-8.60 (1H, m), 8.65-8.80 (1H, m)

MASS (API-ES, Pos): 692.3 (M+H)⁺, 714.3 (M+Na)⁺(free)

### (18) 3-[[(6R, 9aR)-6-Benzhydryl-8-[[2-ethoxy-4-methoxy-6-(2,2,2-trifluoroethoxy)-5-pyrimidinyl]methyl]octahydro-2H-pyrazino[1,2-a]pyrazin-2-yl]carbonyl]-2-pyridinamine Trihydrochloride

NMR (DMSO-d₆, δ): 1.34 (3H, t, J=7.0 Hz), 1.90-4.35 (18H, m), 4.38 (2H, q, J=7.0 Hz), 4.75-5.05 (2H, m), 6.85-7.00 (1H, m), 7.10-7.50 (11H, m), 7.60-8.00 (2H, m), 8.00-8.15 (1H, m)

MASS (API-ES, Pos): 692.2 (M+H)⁺, 714.1 (M+Na)⁺(free)

### (19) (4R, 9aR)-4-Benzhydryl-2-[[2-ethoxy-4-methoxy-6-(2,2,2-trifluoroethoxy)-5-pyrimidinyl]methyl]-8-[(1-methyl-1H-imidazol-2-yl)carbonyl]octahydro-2H-pyrazino[1,2-a]-pyrazine Dihydrochloride

NMR (DMSO-d₆, δ): 1.34 (3H, t, J=7.0 Hz), 1.90-4.35 (21H, m), 4.37 (2H, q, J=7.0 Hz), 4.75-5.05 (2H, m), 7.05-7.60 (12H, m)

MASS (API-ES, Pos): 680.3 (M+H)⁺, 702.2 (M+Na)⁺(free)

### (20) (4R, 9aR)-4-Benzhydryl-8-benzoyl-2-[[2-ethoxy-4-methoxy-6-(2,2,2-trifluoroethoxy)-5-pyrimidinyl]methyl]-octahydro-2H-pyrazino[1,2-a]pyrazine Dihydrochloride

NMR (DMSO-d₆, δ): 1.25-1.45 (3H, m), 1.90-4.50 (20H, m), 4.75-5.10 (2H, m), 7.00-7.65 (15H, m)

MASS (API-ES, Pos): 676.3 (M+H)⁺, 698.2 (M+Na)⁺(free)

### (21) (4R, 9aR)-4-Benzhydryl-2-[[2-ethoxy-4-methoxy-6-(2,2,2-trifluoroethoxy)-5-pyrimidinyl]methyl]-8-(5-pyrimidinylcarbonyl)octahydro-2H-pyrazino[1,2-a]-pyrazine Dihydrochloride

NMR (DMSO-d₆, δ): 1.33 (3H, t, J=6.7 Hz), 1.90-4.50 (20H, m), 4.75-5.10 (2H, m), 7.05-7.55 (10H, m), 8.70-9.00 (2H, m), 9.28 (1H, s)

MASS (API-ES, Pos): 678.3 (M+H)⁺(free)

### (22) (4R, 9aR)-4-Benzhydryl-2-[[2-ethoxy-4-methoxy-6-(2,2,2-trifluoroethoxy)-5-pyrimidinyl]methyl]-8-(4-pyrimidinylcarbonyl)octahydro-2H-pyrazino[1,2-a]-pyrazine Dihydrochloride

NMR (DMSO-d₆, δ): 1.25-1.45 (3H, m), 1.90-4.50 (20H, m), 4.75-5.05 (2H, m), 7.10-7.50 (10H, m), 7.60-7.75 (1H, m), 8.90-9.05 (1H, m), 9.15-9.30 (1H, m)

MASS (API-ES, Pos): 678.3 (M+H)⁺, 700.3 (M+Na)⁺(free)

### (23) (4R, 9aR)-4-Benzhydryl-2-[[2-ethoxy-4-methoxy-6-(2,2,2-trifluoroethoxy)-5-pyrimidinyl]methyl]-8-(2-pyrimidinylcarbonyl)octahydro-2H-pyrazino[1,2-a]-pyrazine Dihydrochloride

NMR (DMSO-d₆, δ): 1.25-1.45 (3H, m), 1.90-4.50 (20H, m), 4.80-5.10 (2H, m), 7.05-7.50 (10H, m), 7.55-7.70 (1H, m), 8.70-9.00 (2H, m)

MASS (API-ES, Pos): 678.3 (M+H)⁺, 700.3 (M+Na)⁺(free)

### (24) (4R, 9aR)-4-Benzhydryl-2-[[2-ethoxy-4-methoxy-6-(2,2,2-trifluoroethoxy)-5-pyrimidinyl]methyl]-8-(tetrahydro-2H-pyran-4-ylcarbonyl)octahydro-2H-pyrazino[1,2-a]-pyrazine Dihydrochloride

NMR (DMSO-d₆, δ): 1.25-1.80 (7H, m), 1.90-4.60 (25H, m), 4.75-5.10 (2H, m), 7.05-7.60 (10H, m)

MASS (API-ES, Pos): 684.2 (M+H)⁺, 706.2 (M+Na)⁺(free)

### (25) (4R, 9aR)-4-Benzhydryl-2-[[2-ethoxy-4-methoxy-6-(2,2,2-trifluoroethoxy)-5-pyrimidinyl]methyl]-8-[(3-methyl-3-oxetanyl)carbonyl]octahydro-2H-pyrazino[1,2-a]-pyrazine Dihydrochloride

NMR (DMSO-d₆, δ): 1.33 (3H, t, J=7.1 Hz), 1.40-1.55 (3H, m), 2.10-4.30 (20H, m), 4.38 (2H, q, J=7.0 Hz), 4.60-5.05 (4H, m), 7.10-7.50 (10H, m)

MASS (API-ES, Pos): 670.2 (M+H)⁺, 692.2 (M+Na)⁺(free)

### (26) (4R, 9aR)-4-Benzhydryl-8-(1,3-dioxan-5-ylcarbonyl)-2-[[2-ethoxy-4-methoxy-6-(2,2,2-trifluoroethoxy)-5-pyrimidinyl]methyl]octahydro-2H-pyrazino[1,2-a]-pyrazine Dihydrochloride

NMR (DMSO-d₆, δ): 1.33 (3H, t, J=7.1 Hz), 2.05-4.30 (23H, m), 4.38 (2H, q, J=6.9 Hz), 4.50-4.65 (1H, m), 4.80-5.05 (3H, m), 7.10-7.50 (10H, m)

MASS (API-ES, Pos): 686.3 (M+H)⁺, 708.0 (M+Na)⁺(free)

### (27) 2-[[(6R, 9aR)-6-Benzhydryl-8-[[2-ethoxy-4-methoxy-6-(2,2,2-trifluoroethoxy)-5-pyrimidinyl]methyl]octahydro-2H-pyrazino[1,2-a]pyrazin-2-yl]carbonyl]-1,3-propanediol Dihydrochloride

NMR (DMSO-d₆, δ): 1.34 (3H, t, J=7.1 Hz), 1.90-4.35 (25H, m), 4.38 (2H, q, J=7.0 Hz), 4.80-5.10 (2H, m), 7.05-7.55 (10H, m)

MASS (API-ES, Pos): 674.3 (M+H)⁺, 696.2 (M+Na)⁺(free)

### (28) (4R, 9aR)-4-Benzhydryl-2-[[2-ethoxy-4-methoxy-6-(2,2,2-trifluoroethoxy)-5-pyrimidinyl]methyl]-8-(2-furoyl)octahydro-2H-pyrazino[1,2-a]pyrazine Dihydrochloride

NMR (DMSO-d₆, δ): 1.33 (3H, t, J=7.0 Hz), 2.20-4.20 (17H, m), 4.30 (1H, br s), 4.73 (2H, q, J=7.0 Hz), 4.80-5.00 (2H, m), 6.55-6.70 (1H, m), 6.95-7.05 (1H, m), 7.10-7.50 (10H, m), 7.83 (1H, s)

MASS (APCI, Pos): 666.27 (M+H)⁺(free)

### (29) (4R, 9aR)-4-Benzhydryl-2-[[2-ethoxy-4-methoxy-6-(2,2,2-trifluoroethoxy)-5-pyrimidinyl]methyl]-8-(3-furoyl)octahydro-2H-pyrazino[1,2-a]pyrazine Dihydrochloride

NMR (DMSO-d₆, δ): 1.34 (3H, t, J=7.0 Hz), 2.20-4.15 (17H, m), 4.31 (1H, br s), 4.38 (2H, q, J=7.0 Hz), 4.80-5.05 (2H, m), 6.63 (1H, s), 7.10-7.45 (10H, m), 7.75 (1H, s), 8.01 (1H, s)

MASS (APCI, Pos): 666.20 (M+H)⁺(free)

### (30) 2-[[(6R, 9aR)-6-Benzhydryl-8-[[2-ethoxy-4-methoxy-6-(2,2,2-trifluoroethoxy)-5-pyrimidinyl]methyl]octahydro-2H-pyrazino[1,2-a]pyrazin-2-yl]carbonyl]-2-methyl-1,3-propanediol Dihydrochloride

NMR (DMSO-d₆, δ): 1.03 (3H, s), 1.34 (3H, t, J=7.0 Hz), 2.10-4.35 (24H, m), 4.38 (2H, q, J=6.9 Hz), 4.80-5.10 (2H, m), 7.10-7.50 (10H, m)

MASS (API-ES, Pos): 668.2 (M+H)⁺, 710.2 (M+Na)⁺(free)

### (31) (4R, 9aR)-4-Benzhydryl-2-[[2-ethoxy-4-methoxy-6-(2,2,2-trifluoroethoxy)-5-pyrimidinyl]methyl]-8-[(3-ethyl-3-oxetanyl)carbonyl]octahydro-2H-pyrazino[1,2-a]pyrazine Dihydrochloride

NMR (DMSO-d₆, δ): 0.70-0.95 (3H, m), 1.34 (3H, t, J=7.0 Hz), 1.70-4.35 (22H, m), 4.38 (2H, q, J=7.0 Hz), 4.60-4.85 (2H, m), 4.85-5.00 (2H, m), 7.10-7.45 (10H, m)

MASS (API-ES, Pos): 684.2 (M+H)⁺, 706.3 (M+Na)⁺(free)

### (32) (4R, 9aS)-4-Benzhydryl-2-[[2-ethoxy-4-methoxy-6-(2,2,2-trifluoroethoxy)-5-pyrimidinyl]methyl]-8-[(3-methoxy-2-pyrazinyl)carbonyl]octahydro-2H-pyrazino[1,2-a]pyrazine Dihydrochloride

NMR (CDCl₃, δ): 1.37-1.46 (3H, m), 1.80-2.04 (3H, m), 2.40-2.63 (2H, m), 2.74-3.11 (5H, m), 3.20-3.35 (1H, m), 3.35 and 3.42 (total 3H, each s), 3.71 and 3.77 (total 3H, each s), 3.93-3.99 (total 3H, each s), 4.06-4.14 (1H, m), 4.33-4.39 (2H, m), 4.57-4.67 (2H, m), 7.12-7.28 (10H, m), 8.09 (1H, s), 8.14 (1H, s)

MASS (ES positive): 708 (M+H)⁺

### (33) (4R, 9aS)-4-Benzhydryl-2-[[2-ethoxy-4-methoxy-6-(2,2,2-trifluoroethoxy)-5-pyrimidinyl]methyl]-8-[(4-oxido-2-pyrazinyl)carbonyl]octahydro-2H-pyrazino[1,2-a]pyrazine Dihydrochloride

NMR (CDCl₃, δ): 1.38-1.46 (3H, m), 1.87-2.04 (3H, m), 2.44-2.51 (2H, m), 2.73-3.42 (7H, m), 3.65-3.85 (1H, m), 3.73 and 3.76 (total 3H, each s), 4.08-4.13 (1H, m), 4.30-4.39 (3H, m), 4.57-4.68 (2H, m), 7.13-7.28 (10H, m), 8.02-8.09 (1H, m), 8.31-8.39 (2H, m)

MASS (ES positive): 694 (M+H)⁺

### (34) (4R, 9aR)-4-Benzhydryl-2-[[2-ethoxy-4-methoxy-6-(2,2,2-trifluoroethoxy)-5-pyrimidinyl]methyl]-8-[(5-methyl-4-oxido-2-pyrazinyl)carbonyl]octahydro-2H-pyrazino-[1,2-a]pyrazine Dihydrochloride

NMR (CDCl₃, δ): 1.39-1.46 (3H, m), 1.86-2.04 (3H, m), 2.44-2.57 (5H, m), 2.77-3.42 (7H, m), 3.73-3.90 (4H, m), 4.07-4.39 (4H, m), 4.57-4.68 (2H, m), 7.16-7.28 (10H, m), 8.31-8.44 (2H, m)

MASS (ES positive): 708 (M+H)⁺

### (35) (4R, 9aR)-4-Benzhydryl-2-[[2,4-dimethoxy-6-(2,2,2-trifluoroethoxy)-5-pyrimidinyl]methyl]-8-[(3-methyl-3-oxetanyl)carbonyl]octahydro-2H-pyrazino[1,2-a]pyrazine Dihydrochloride

NMR (DMSO-d₆, δ): 1.40-1.55 (3H, m), 2.10-4.40 (23H, m), 4.60-5.05 (4H, m), 7.10-7.40 (10H, m)

MASS (API-ES, Pos): 656.2 (M+H)⁺, 678.2 (M+Na)⁺(free)

### (36) (4R, 9aR)-4-Benzhydryl-2-[(2-cyclopropyl-4-isopropoxy-6-methoxy-5-pyrimidinyl)methyl]-8-[(3-methyl-3-oxetanyl)-carbonyl]octahydro-2H-pyrazino[1,2-a]pyrazine Dihydrochloride

NMR (DMSO-d₆, δ): 0.80-1.30 (10H, m), 1.40-1.55 (3H, m), 1.90-4.10 (17H, m), 4.10-4.40 (4H, m), 4.60-4.80 (2H, m), 5.10-5.20 (1H, m), 7.10-7.40 (10H, m)

MASS (API-ES, Pos): 626.3 (M+H)⁺, 648.2 (M+Na)⁺(free)

### (37) (4R, 9aR)-4-Benzhydryl-2-[(2-cyclopropyl-4-isopropoxy-6-methoxy-5-pyrimidinyl)methyl]-8-[(3-ethyl-3-oxetanyl)-carbonyl]octahydro-2H-pyrazino[1,2-a]pyrazine Dihydrochloride

NMR (DMSO-d₆, δ): 0.60-1.35 (13H, m), 1.70-4.10 (19H,m), 4.10-4.40 (4H, m), 4.60-4.85 (2H, m), 5.05-5.25 (1H, m), 7.10-7.60 (10H, m)

MASS (API-ES, Pos): 610.4 (M+H)⁺(free)

### (38) (4R, 9aR)-4-Benzhydryl-2-[[2,4-dimethoxy-6-(2,2,2-trifluoroethoxy)-5-pyrimidinyl]methyl]-8-(1,3-dioxan-5-ylcarbonyl)octahydro-2H-pyrazino[1,2-a]pyrazine Dihydrochloride

NMR (CDCl₃, δ): 2.05-2.30 (1H, m), 2.30-4.15 (24H, m), 4.15-4.35 (1H, m), 4.50-4.65 (1H, m), 4.85-5.10 (3H, m), 7.15-7.55 (10H, m)

MASS (API-ES, Pos): 672.2 (M+H)⁺, 694.3 (M+Na)⁺(free)

### (39) (4R, 9aR)-4-Benzhydryl-2-[(2-cyclopropyl-4-isopropoxy-6-methoxy-5-pyrimidinyl)methyl]-8-(1,3-dioxan-5-ylcarbonyl)octahydro-2H-pyrazino[1,2-a]pyrazine Dihydrochloride

NMR (CDCl₃, δ): 0.95-1.10 (4H, m), 1.10-1.30 (6H, m), 1.90-4.40 (24H, m), 4.55-4.65 (1H, m), 4.90-5.00 (1H, m), 5.10-5.20 (1H, m), 7.10-7.45 (10H, m)

MASS (API-ES, Pos): 642.3 (M+H)⁺(free)

### EXAMPLE 13

To a solution of (4R, 9aS)-4-benzhydryl-2-[[2-ethoxy-4-methoxy-6-(2,2,2-trifluoroethoxy)-5-pyrimidinyl]methyl]-octahydro-2H-pyrazino[1,2-a]pyrazine in 0.5 ml of dichloromethane was added N,N-diisopropylethylamine and morpholine-4-carbonyl chloride, and stirred for 2 hours at ambient temperature. The reaction mixture was added 2 ml of saturated sodium hydrogen carbonate aqueous solution, then extracted with dichloromethane (5 ml×3). The organic layer was dried over diatomaceous earth and evaporated under reduced pressure to give a crude oil. Purification by preparative TLC (0.5 mm silica gel, ethyl acetate) gave pale yellow oil. To a solution of the oil in 2 ml of ethyl acetate was added 0.5 ml of 4N hydrogen chloride/ethyl acetate at 0° C., then the volatiles were evaporated in vacuo to give (4R, 9aR)-4-benzhydryl-2-[[2-ethoxy-4-methoxy-6-(2,2,2-trifluoroethoxy)-5-pyrimidinyl]methyl]-8-(4-morpholinylcarbonyl)octahydro-2H-pyrazino[1,2-a]pyrazine dihydrochloride as a pale yellow solid.

NMR (DMSO-d₆, δ): 1.34 (3H, t, J=7.0 Hz), 1.90-4.35 (28H, m), 4.37 (2H, q, J=7.0 Hz), 7.10-7.55 (10H, m)

MASS (API-ES, Pos): 685.3 (M+H)⁺, 707.0 (M+Na)⁺(free)

### EXAMPLE 14

The following compounds were obtained according to a similar manner to that of Example 13.

### (1) (6R, 9aR)-6-Benzhydryl-8-[[2-ethoxy-4-methoxy-6-(2,2,2-trifluoroethoxy)-5-pyrimidinyl]methyl]-N,N-dimethyloctahydro-2H-pyrazino[1,2-a]pyrazine-2-carboxamide Dihydrochloride

NMR (DMSO-d₆, δ): 1.34 (3H, t, J=7.0 Hz), 1.90-4.35 (24H, m), 4.38 (2H, q, J=7.0 Hz), 4.80-5.05 (2H, m), 7.10-7.50 (10H, m)

MASS (API-ES, Pos): 643.3 (M+H)⁺, 666.3 (M+Na)⁺(free)

### (2) (4R, 9aR)-6-Benzhydryl-2-[[2-ethoxy-4-methoxy-6-(2,2,2-trifluoroethoxy)-5-pyrimidinyl]methyl]-8-[(4-methyl-1-piperazinyl)carbonyl]octahydro-2H-pyrazino[1,2-a]-pyrazine Triihydrochloride

NMR (DMSO-d₆, δ): 1.34 (3H, t, J=7.0 Hz), 1.90-4.35 (29H, m), 4.38 (2H, q, J=7.0 Hz), 4.80-5.05 (2H, m), 7.10-7.50 (10H, m)

MASS (API-ES, Pos): 698.3 (M+H)⁺(free)

### (3) (6R, 9aR)-6-Benzhydryl-8-[[2-ethoxy-4-methoxy-6-(2,2,2-trifluoroethoxy)-5-pyrimidinyl]methyl]-N,N-diethyloctahydro-2H-pyrazino[1,2-a]pyrazine-2-carboxamide Dihydrochloride

NMR (DMSO-d₆, δ): 1.01 (6H, t, J=6.7 Hz), 1.34 (3H, t, J=6.9 Hz), 1.90-4.60 (24H, m), 4.80-5.05 (2H, m), 7.05-7.55 (10H, m)

MASS (API-ES, Pos): 671.3 (M+H)⁺, 693.2 (M+Na)⁺(free)

### (4) (6R, 9aR)-6-Benzhydryl-8-[[2-ethoxy-4-methoxy-6-(2,2,2-trifluoroethoxy)-5-pyrimidinyl]methyl]-N,N-diisopropyloctahydro-2H-pyrazino[1,2-a]pyrazine-2-carboxamide Dihydrochloride

NMR (DMSO-d₆, δ): 1.15 (12H, d, J=6.7 Hz), 1.34 (3H, t, J=7.0 Hz), 1.90-4.35 (20H, m), 4.38 (2H, q, J=7.0 Hz), 4.85-5.05 (2H, m), 7.10-7.50 (10H, m)

MASS (API-ES, Pos): 699.2 (M+H)⁺(free)

### (5) (4R, 9aR)-4-Benzhydryl-2-[[2-ethoxy-4-methoxy-6-(2,2,2-trifluoroethoxy)-5-pyrimidinyl]methyl]-8-(1-pyrrolidinylcarbonyl)octahydro-2H-pyrazino[1,2-a]-pyrazine Dihydrochloride

NMR (DMSO-d₆, δ): 1.34 (3H, t, J=7.1 Hz), 1.72 (4H, s), 1.90-4.80 (24H, m), 4.85-5.05 (2H, m), 7.15-7.50 (10H, m)

MASS (API-ES, Pos): 669.2 (M+H)⁺, 691.3 (M+Na)⁺(free)

### (6) (4R, 9aR)-4-Benzhydryl-2-[[2-ethoxy-4-methoxy-6-(2,2,2-trifluoroethoxy)-5-pyrimidinyl]methyl]-8-(1-piperidinylcarbonyl)octahydro-2H-pyrazino[1,2-a]-pyrazine Dihydrochloride

NMR (DMSO-d₆, δ): 1.34 (3H, t, J=7.0 Hz), 1.35-1.65 (6H, m), 1.90-4.70 (24H, m), 4.93 (2H, q, J=8.8 Hz), 7.10-7.45 (10H, m)

MASS (API-ES, Pos): 683.3 (M+H)⁺, 705.3 (M+Na)⁺(free)

1. A compound of the formula (I): 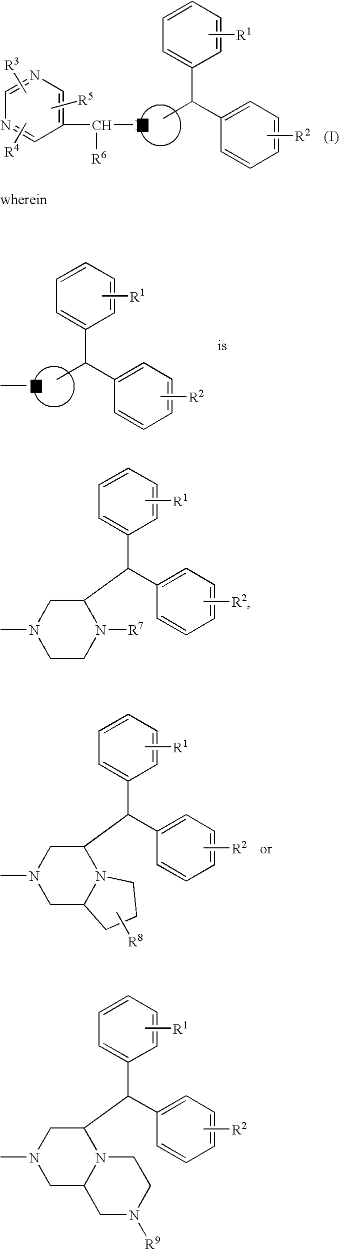
in which
R¹ and R² are independently hydrogen, halogen or lower alkyl,
R⁷ is hydrogen, lower alkoxycarbonyl or pyrazolyl(lower)alkyl optionally substituted with lower alkyl,
R⁸ is hydrogen or lower alkanoylamino,
R⁹ is hydrogen; lower alkanoyl optionally substituted with one, two or three substituent(s) selected from a group consisting of hydroxy, lower alkoxy, halogen, hydroxy(lower)alkyl, acetylamino, mono(or di)(lower)alkylamino and pyridyl; di(lower)alkylcarbamoyl; bis(hydroxy(lower)alkyl)carbamoyl; benzyloxycarbonyl; benzoyl; or heterocycliccarbonyl optionally substituted with one or two substituent(s) selected, from a group consisting of hydroxy, lower alkyl, lower alkoxy, amino, carbamoyl and oxido,
R³, R⁴ and R⁵ are independently hydrogen; halogen; lower alkyl; cyclo(lower)alkyl; cyclo(lower)alkyloxy; lower alkoxy optionally substituted with one, two or three halogen(s); or lower alkanoyl optionally substituted with one, two or three halogen(s), and
R⁶ is hydrogen or lower alkyl,
and a salt thereof.

2. The compound of claim 1, in which 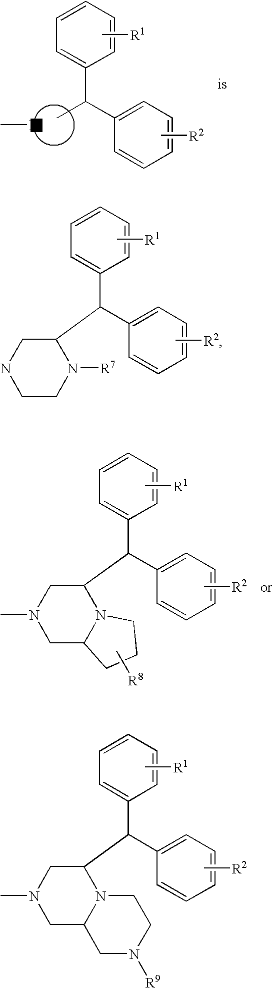
in which
R¹ and R² are independently hydrogen, halogen or lower alkyl,
R⁷ is hydrogen, lower alkoxycarbonyl or pyrazolyl(lower)alkyl optionally substituted with lower alkyl,
R⁸ is hydrogen or lower alkanoylamino,
R⁹ is hydrogen; lower alkanoyl optionally substituted with one, two or three substituent(s) selected from a group consisting of hydroxy, halogen, lower alkoxy, hydroxy(lower)alkyl, acetylamino, mono(or di)(lower)alkylamino and pyridyl; di(lower)alkylcarbamoyl; bis(hydroxy(lower)alkyl)carbamoyl; benzyloxycarbonyl; benzoyl; or pyrrolylcarbonyl, imidazolylcarbonyl, pyrazolylcarbonyl, pyridylcarbonyl, pyrimidylcarbonyl, pyrazinylcarbonyl, pyrrolidinylcarbonyl, piperidylcarbonyl, piperazinylcarbonyl, isoxazolylcarbonyl, morpholinylcarbonyl, furylcarbonyl, oxetanylcarbonyl, oxolanylcarbonyl, tetrahydropyranylcarbonyl or dioxanylcarbonyl, each of which may be substituted with one or two substituen(s) selected from a group consisting of hydroxy, lower alkyl, lower alkoxy, carbamoyl, amino and N-oxido,
R³, R⁴ and R⁵ are independently hydrogen; halogen; lower alkyl; cyclo(lower)alkyl; cyclo(lower)alkyloxy; lower alkoxy optionally substituted with one, two or three halogen(s); or lower alkanoyl optionally substituted with one, two or three halogen(s), and
R⁶ is hydrogen or lower alkyl.

3. The compound of claim 2, in which 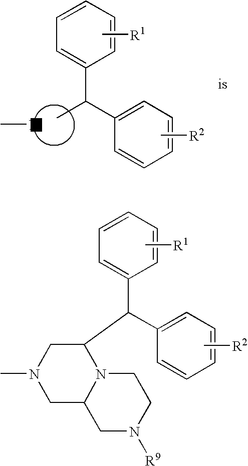
in which
R¹ and R² are each hydrogen,
R⁹ is hydrogen; lower alkanoyl optionally substituted with one, two or three substituent(s) selected from a group consisting of hydroxy, halogen, lower alkoxy, hydroxy(lower)alkyl, acetylamino, mono(or di)(lower)alkylamino and pyridyl; di(lower)alkylcarbamoyl; bis(hydroxy(lower)alkyl)carbamoyl; benzyloxycarbonyl; benzoyl; or pyrrolylcarbonyl, imidazolylcarbonyl, pyrazolylcarbonyl, pyridylcarbonyl, pyrimidylcarbonyl, pyrazinylcarbonyl, pyrrolidinylcarbonyl, piperidylcarbonyl, piperazinylcarbonyl, isoxazolylcarbonyl, morpholinylcarbonyl, furylcarbonyl, oxetanylcarbonyl, oxolanylcarbonyl, tetrahydropyranylcarbonyl or dioxanylcarbonyl, each of which may be substituted with one or two substituen(s) selected from a group consisting of hydroxy, lower alkyl, lower alkoxy, carbamoyl, amino and N-oxido,
R³, R⁴ and R⁵ are independently hydrogen; halogen; lower alkyl; cyclo(lower)alkyl; cyclo(lower)alkyloxy; lower alkoxy optionally substituted with one, two or three halogen(s); or lower alkanoyl optionally substituted with one, two or three halogen(s), and
R⁶ is hydrogen.

4. A compound of claim 3, which is selected from a group consisting of
(1) 2-(6R, 9aR)-6-benzhydryl-8-[(2,4,6-trimethoxy-5-pyrimidinyl)methyl]octahydro-2H-pyrazino[1,2-a]-pyrazin-2-yl]-2-oxoethanol,
(2) (4R, 9aR)-4-benzhydryl-2-[(2-cyclopropyl-4-isopropoxy-6-methoxy-5-pyrimidinyl)methyl]-8-(methoxyacetyl)octahydro-2H-pyrazino[1,2-a]pyrazine,
(3) (4R, 9aR)-4-benzhydryl-2-[(2-ethoxy-4-isopropoxy-6-methoxy-5-pyrimidinyl)methyl]-8-(3-methoxypropanoyl)octahydro-2H-pyrazino[1,2-a]-pyrazine,
(4) (4R, 9aR)-4-benzhydryl-2-[(2-cyclopropyl-4-isopropoxy-6-methoxy-5-pyrimidinyl)methyl]-8-(3-methoxypropanoyl)octahydro-2H-pyrazino[1,2-a]-pyrazine,
(5) (4R, 9aR)-4-benzhydryl-2-[(2-cyclopropyl-4-ethoxy-6-methoxy-5-pyrimidinyl)methyl]-8-(3-methoxypropanoyl)octahydro-2H-pyrazino[1,2-a]-pyrazine,
(6) (4R, 9aR)-4-Benzhydryl-2-[[2-ethoxy-4-methoxy-6-(2,2,2-trifluoroethoxy)-5-pyrimidinyl]methyl]-8-(3-methoxypropanoyl)octahydro-2H-pyrazino[1,2-a]-pyrazine,
(7) (4R, 9aR)-4-Benzhydryl-2-[(2-ethoxy-4-isopropoxy-6-methoxy-5-pyrimidinyl)methyl]-8-(2-pyrazinylcarbonyl)octahydro-2H-pyrazino[1,2-a]-pyrazine,
(8) (4R, 9aR)-4-Benzhydryl-2-[[2-cyclopropyl-4-methoxy-6-(2,2,2-trifluoroethoxy)-5-pyrimidinyl]methyl]-8-(2-pyrazinylcarbonyl)octahydro-2H-pyrazino[1,2-a]-pyrazine,
or a pharmaceutically acceptable salt thereof.

5. A process for the preparation of the compound of claim 1 or a salt thereof, which comprises,
(1) reacting a compound of the formula (II): 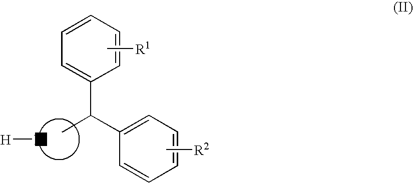
wherein 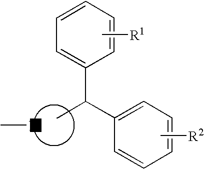
is as defined in claim 1, or its reactive derivative at the imino group or a salt thereof, with a compound of the formula (III): 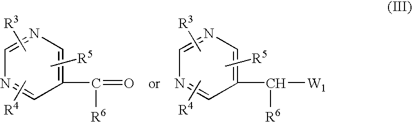
wherein R³, R⁴, R⁵ and R⁶ are each as defined in claim 1 and
W₁ is a leaving group,
or a salt thereof to give a compound of the formula (I): 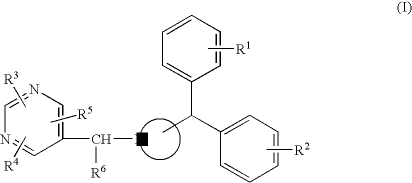
wherein 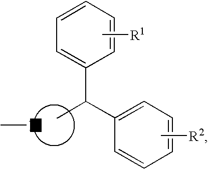
R³, R⁴, R⁵ and R⁶ are each as defined in claim 1,
or a salt thereof, or
(2) reacting a compound of the formula (Ia): 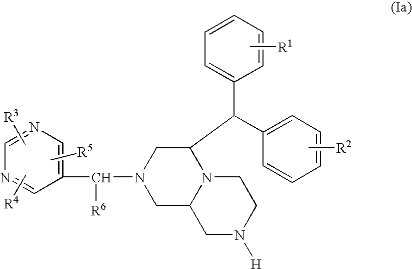
wherein R¹, R², R³, R⁴, R⁵ and R⁶ are each as defined in claim 1,
or a salt thereof, with a compound of the formula (IV):
W₂—R⁹ (IV)
wherein R⁹ is as defined in claim 1, and
W₂ is a leaving group,
or a salt thereof to give a compound of the formula (Ib): 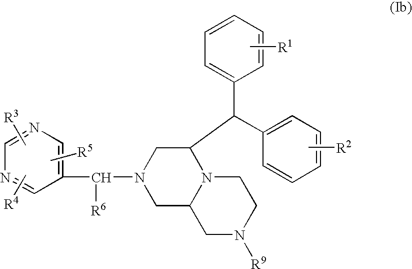
wherein R¹, R², R³, R⁴, R⁵, R⁶ and R⁹ are each as defined in claim 1,
or a salt thereof, or
(3) cyclizing a compound of the formula (V): 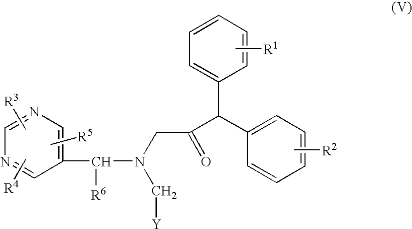
wherein R¹, R², R³, R⁴, R⁵ and R⁶ are each as defined in claim 1,
and
Y is 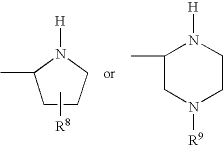
wherein R⁸ and R⁹ are each as defined in claim 1, or its reactive derivative at the imino group or a salt thereof, to give a compound of the formula (Ic): 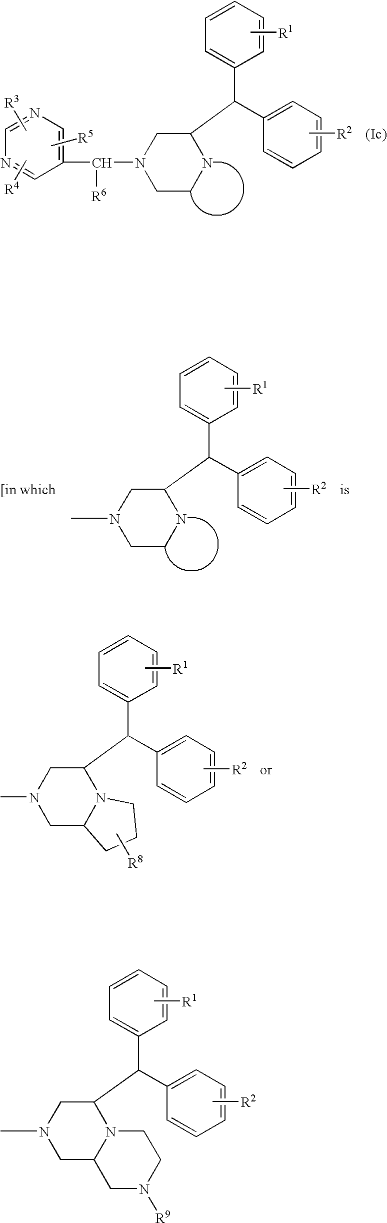
wherein R¹, R², R⁸ and R⁹ are each as defined in claim 1,
or a salt thereof.

6. A pharmaceutical composition which comprises, as an active ingredient, a compound of claim 1 or a pharmaceutically acceptable salt thereof in admixture with pharmaceutically acceptable carriers.

7. A compound of claim 1 for use as a medicament.

8. A method for treating or preventing Tachykinin-mediated diseases which comprises administering an effective amount of a compound of claim 1 or a pharmaceutically acceptable salt thereof to human being or animals.

9. A compound of claim 1 for use as Tachykinin antagonist.

10. Use of a compound of claim 1 for manufacture of a medicament for treating or preventing Tachykinin-mediated diseases.
